(19) United States
(12) Patent Application Publication
(10) Pub. No.: US 2022/0340976 A1
(43) Pub. Date: Oct. 27, 2022

(54) RAPID PREDICTION OF DRUG RESPONSIVENESS

(71) Applicants: THE BROAD INSTITUTE, INC., Cambridge, MA (US); DANA-FARBER CANCER INSTITUTE, INC., Boston, MA (US)

(72) Inventors: Andrew J. Aguirre, Boston, MA (US); Francisca Vazquez, Cambridge, MA (US); Aviad Tsherniak, Cambridge, MA (US); James McFarland, Cambridge, MA (US); Brenton Paolella, Cambridge, MA (US)

(73) Assignees: THE BROAD INSTITUTE, INC., Cambridge, MA (US); DANA-FARBER CANCER INSTITUTE, INC., Boston, MA (US)

(21) Appl. No.: 17/639,532

(22) PCT Filed: Sep. 1, 2020
(86) PCT No.: PCT/US20/48896

§ 371 Date: Mar. 1, 2022

Related U.S. Application Data
(60) Provisional application No. 62/894,924, filed on Sep. 2, 2019.

(51) Int. Cl. C12Q 1/6886; G06N 20/00; G06N 3/08

(52) U.S. Cl. CPC C12Q 1/6886; G06N 20/00; G06N 3/08; C12Q 2600/106

(57) ABSTRACT

The present disclosure relates to compositions and methods for rapid prediction, based upon early single cell transcriptomic assessment of a biopsied sample obtained from a subject, of whether a subject will be responsive to a drug.

### CROSS REFERENCE TO RELATED APPLICATION

This application claims the benefit of U.S. Provisional Application No. 62/894,924, filed Sep. 2, 2019, entitled “Rapid Prediction of Drug Responsiveness.” The entire contents of the aforementioned application are incorporated herein by reference.

### FIELD OF THE INVENTION

The current disclosure relates to methods for rapidly identifying, optionally prior to treatment, responsiveness of a cell, tissue and/or organism to one or more drugs.

### BACKGROUND OF THE INVENTION

For the majority of cancer patients, there are no standard biomarker-driven strategies to date to guide the choice of treatment regimens. Empiric therapy with highly toxic regimens leads to significant morbidity from cancer treatments, while only a subset of patients achieve a therapeutic benefit. Although a small subset of cancer patients possess clinically relevant genomic lesions, the majority of patients do not harbor actionable alterations; and additional strategies for therapeutic selection would be particularly useful to guide treatments for these patients. A need therefore exists in translational oncology research for rapid turnaround assays capable of accurately predicting treatment response of a subject from a small biopsy sample.

### BRIEF SUMMARY OF THE INVENTION

The current disclosure relates, at least in part, to the discovery of methods for performing rapid and reliable drug-response profiling upon a biopsy sample of a subject, via use of bulk or single cell transcriptional profiling and comparison of such transcriptional profiles to known drug-responsive transcriptional signatures. Advantageously, the methods of the instant disclosure can be performed with robust predictive accuracy within hours of obtaining a biopsy sample, and can even be performed upon a heterogeneous population of biopsy cells containing both tumor and associated stromal cells (e.g. immune, fibroblasts).

In one aspect, the instant disclosure provides a method for selecting a drug for treatment of a subject having or at risk of developing a disease or disorder, the method involving: a) obtaining a population of cells from the subject; b) contacting the population of cells with a test drug; c) obtaining one or more single cell transcriptional profiles of the population of cells from the subject; d) comparing the one or more single cell transcriptional profiles of the population of cells from the subject with a reference test drug-responsive transcriptional signature; e) identifying a match between the one or more single cell transcriptional profiles of the population of cells from the subject and the reference test drug-responsive transcriptional signature; and f) selecting the test drug for administration to the subject.

In one embodiment, the one or more single cell transcriptional profiles are obtained within a week of obtaining the population of cells from the subject. Optionally, the single cell transcriptional profiles are obtained within 48 hours of obtaining the population of cells from the subject, or within 24 hours of obtaining the population of cells from the subject, or in a 3-24 hour time window after obtaining the population of cells from the subject.

In certain embodiments, the one or more single cell transcriptional profiles are obtained at multiple timepoints. Optionally, the one or more single cell transcriptional profiles are obtained at two or more timepoints between 3 and 48 hours after step (b) (contacting the population of cells with the test drug).

In some embodiments, the population of cells of step (a) is obtained from a needle biopsy and/or a tumor biopsy. Optionally, the biopsy includes a heterogeneous (mixed) population of cells. Optionally, the biopsy contains between about 25,000 and about 50,000 cells.

In embodiments, the test drug is a small molecule, a nucleic acid or a peptide.

In certain embodiments, the test drug is dabrafenib, trametinib, bortezomib, nutlin, navitoclax, everolimus, CGS15943, AZD5591, afatinib, JQ1, gemcitabine, taselisib and/or prexasertib. In some embodiments, the test drug may also include compounds that fall into classes of drugs that include cytotoxic chemotherapies, targeted signaling pathway inhibitors such as EGFR inhibitors or KRAS inhibitors, anti-hormonal therapies such as anti-androgens or anti-estrogens, DNA damage repair inhibitors or epigenetic inhibitors (e.g., Dnmt2 inhibitors, HDAC inhibitors, etc.).

In some embodiments, the subject has a neoplasia. Optionally, the neoplasia is a carcinoma or sarcoma of the head, neck, lung, esophagus, stomach, small intestine, pancreas, gall bladder, biliary ducts, liver, kidney, adrenal gland, colon, rectum, anus, skin, connective tissues, blood vessels, muscle, bone or brain. In certain embodiments, the subject has a cancer of the blood, a cancer of the bone marrow, a cancer of the lymph nodes, a cancer of the spleen and/or a cancer of the immune system.

In some embodiments, the subject has a non-cancerous neoplastic condition. Optionally, the non-cancerous neoplastic condition is a hyperproliferative blood cell condition (for example including, but not limited to, a myeloproliferative neoplasm, an eosinophilic syndrome, Sweet's syndrome, Hemophagocytic lymphohistiocytosis (HLH) and related conditions).

In embodiments, the instant the approach may be applied to microbial conditions involving ex vivo therapy to bacterial or vival cultures to evaluate antibiotic susceptibility.

In embodiments, the reference test drug-responsive transcriptional signature is obtained from transcriptome sequencing of known test drug-responsive cell lines or test drug-responsive organoids.

In certain embodiments, the reference test drug-responsive transcriptional signature is obtained from transcriptome profiling of known test drug-responsive cell lines at multiple timepoints after administration of test drug.

In some embodiments, the reference test drug-responsive transcriptional signature is obtained or refined using machine learning.

In embodiments, identifying a match between the one or more single cell transcriptional profiles of the population of cells from the subject and the reference test drug-responsive transcriptional signature in step (e) involves comparing one or more principal components of the reference test drug-responsive transcriptional signature with the one or more single cell transcriptional profiles of the population of cells from the subject and identifying the one or more principal components of the reference test drug-responsive transcriptional signature in the one or more single cell transcriptional profiles of the population of cells from the subject. In a related embodiment, a single principal component is identified as a match between the reference test drug-responsive transcriptional signature and the one or more single cell transcriptional profiles of the population of cells from the subject. In another related embodiment, two or more principal components are identified as a match between the reference test drug-responsive transcriptional signature and the one or more single cell transcriptional profiles of the population of cells from the subject. Optionally, three or more principal components are identified as a match between the reference test drug-responsive transcriptional signature and the one or more single cell transcriptional profiles of the population of cells from the subject. Optionally, four or more principal components are identified as a match between the reference test drug-responsive transcriptional signature and the one or more single cell transcriptional profiles of the population of cells from the subject.

In certain embodiments, a selection of principal components of the reference test drug-responsive transcriptional signature is identified in the one or more single cell transcriptional profiles of the population of cells from the subject.

In some embodiments, all identified principal components of the reference test drug-responsive transcriptional signature are also identified in the one or more single cell transcriptional profiles of the population of cells from the subject.

In embodiments, the test drug is selected for administration to the subject if about 25% or more of the single cell transcriptional profiles obtained from the population of cells from the subject match the reference test drug-responsive transcriptional signature. (It is expressly contemplated that this threshold value can be adjusted by the practitioner, for example, to at least about 5% of the single cell transcriptional profiles, at least about 10% of the single cell transcriptional profiles, at least about 15% of the single cell transcriptional profiles, at least about 20% of the single cell transcriptional profiles, at least about 30% of the single cell transcriptional profiles, at least about 35% of the single cell transcriptional profiles, at least about 40% of the single cell transcriptional profiles, at least about 45% of the single cell transcriptional profiles, at least about 50% of the single cell transcriptional profiles, at least about 55% of the single cell transcriptional profiles, at least about 60% of the single cell transcriptional profiles, at least about 65% of the single cell transcriptional profiles, at least about 70% of the single cell transcriptional profiles, at least about 75% of the single cell transcriptional profiles, or even higher threshold values).

In some embodiments, the one or more single cell transcriptional profiles of the population of cells from the subject are obtained via next-generation sequencing. Optionally, the one or more single cell transcriptional profiles via a Seq-Well, and/or Drop-Seq process. Optionally, the one or more transcriptional profiles are assayed using a more focused assay, such as Nanostring® (see, e.g., U.S. Pat. Nos. 7,473,767; 7,919,237; 7,941,279; 8,148,512; 8,415,102; 8,492,094; 8,519,115; 8,986,926; 9,066,963; 9,181,588; 9,376,712; 9,371,563; 9,920,380; 10,077,466; 9,856,519; and 9,714,446, which disclose, e.g., barcoding technologies that enable spatially resolved, digital readout of proteins and/or RNA targets in multiplexed assays) and/or an in situ hybridization approach.

In certain embodiments, a match with the reference test drug-responsive transcriptional signature indicates diminished cell viability and/or apoptosis of the biopsy cell(s).

In embodiments, the selected test drug is administered to the subject.

In some embodiments, the predictive accuracy of early (e.g., 3-48 hours after biopsy and/or drug administration) single cell transcript profiles obtained from known drug-responsive cells is compared with the predictive accuracy of longer term (either post-drug administration or after isolation/expansion of a biopsy-derived cell line and/or organoid) transcript signatures of known drug-responsive cells. Optionally, the early single cell transcript profiles are approximately as accurate as, or are more accurate than, the longer-term transcript signatures of known drug-responsive cells.

In certain embodiments of the instant disclosure, the early single cell transcript profiles accurately predict whether the subject responds to a selected therapy.

Another aspect of the instant disclosure provides a method for identifying a transcriptional signature for drug-responsive cells, the method involving: a) contacting a population of cells including multiple cell types with a drug, where the multiple cell types are known to differ in their responsiveness to the drug; b) obtaining single cell transcript sequences/expression data from multiple cells of the population of cells; and c) comparing the single cell transcript sequences/expression data obtained from known drug-responsive cell types of the population to single cell transcript sequences/expression data obtained from known non-drug-responsive cell types of the population, thereby identifying transcript sequences/transcript levels that distinguish the known drug-responsive cell types from the known non-drug-responsive cell types; and d) assembling the transcript sequences/transcript levels that distinguish the known drug-responsive cell types from the known non-drug-responsive cell types into a transcriptional signature for drug-responsive cells, thereby identifying a transcriptional signature for drug-responsive cells.

In embodiments, genetic profiling (e.g., SNP profiling) is employed to distinguish between the multiple cell types of the population of cells in comparing the single cell transcript sequences/expression data obtained from known drug-responsive cell types of the population to single cell transcript sequences/expression data obtained from known non-drug-responsive cell types of the population.

In certain embodiments, single cell transcript sequences/expression data are obtained at multiple timepoints after contacting the population of cells with the drug. Optionally, the timepoints are between 3 and 48 hours after contacting the population of cells with the drug.

In some embodiments, the population of cells including multiple cell types is obtained from cell lines and/or ex vivo organoid models.

In certain embodiments, the reference test drug-responsive transcriptional signature and/or transcriptional signature for drug-responsive cells includes a selection of transcripts (e.g., two or more transcripts, three or more transcripts, four or more transcripts, five or more transcripts, six or more transcripts, seven or more transcripts, eight or more transcripts, nine or more transcripts, ten or more transcripts, twenty or more transcripts, thirty or more transcripts, forty or more transcripts, fifty or more transcripts, or all transcripts) encoded by the genes listed in FIG. 25.

In embodiments the reference test drug-responsive transcriptional signature and/or transcriptional signature for drug-responsive cells includes gene expression values/measurements for one or more genes of the following: YPEL5, SBDS, PMAIP1, CDKN1A, RASSF1, SERTAD1, PPP1R15A, RPS15, CCDC85B, MAFF, PTMA, MT.CYB, HBEGF, SESN2, HIST2H2AC, SAT1, PTP4A1, ZFAS1, FAM173A, SNHG15, MAT2A, ATF3, IL11, IL8, H3F3A, PDRG1, MRPL33, SRSF2, DDIT3 and NDUFB10. Optionally, the signature profile, values and/or measurements are scaled or otherwise adjusted relative to an appropriate control. In a related embodiment, the appropriate control includes a gene expression level(s) or measurement(s) for a cell pre-determined not to be responsive to the drug.

### Definitions

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value.

In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Unless otherwise clear from context, all numerical values provided herein are modified by the term “about.”

The term “administration” refers to introducing a substance into a subject. In general, any route of administration may be utilized including, for example, parenteral (e.g., intravenous), oral, topical, subcutaneous, peritoneal, intraarterial, inhalation, vaginal, rectal, nasal, introduction into the cerebrospinal fluid, or instillation into body compartments. In some embodiments, administration is oral. Additionally or alternatively, in some embodiments, administration is parenteral. In some embodiments, administration is intravenous.

By “agent” is meant any small compound (e.g., small molecule), antibody, nucleic acid molecule, or polypeptide, or fragments thereof or cellular therapeutics such as allogeneic transplantation and/or CAR T-cell therapy.

As used herein, the term “biological specimen” is intended to mean one or more cell, tissue, organism or portion thereof. A biological specimen can be obtained from any of a variety of organisms. Exemplary organisms include, but are not limited to, a mammal such as a rodent, mouse, rat, rabbit, guinea pig, ungulate, horse, sheep, pig, goat, cow, cat, dog, primate (i.e. human or non-human primate); a plant such as Arabidopsis thaliana, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a Dictyostelium discoideum; a fungi such as Pneumocystis carinii, Takifugu rubripes, yeast, Saccharomyces cerevisiae or Schizosaccharomyces pombe; or a Plasmodium falciparum. Target nucleic acids can also be derived from a prokaryote such as a bacterium, Escherichia coli, Staphylococci or Mycoplasma pneumoniae; an archae; a virus such as Hepatitis C virus or human immunodeficiency virus; or a viroid. Specimens can be derived from a homogeneous culture or population of the above organisms or alternatively from a collection of several different organisms, for example, in a community or ecosystem.

By “control” or “reference” is meant a standard of comparison. In one aspect, as used herein, “changed as compared to a control” sample or subject is understood as having a level that is statistically different than a sample from a normal, untreated, or control sample. Control samples include, for example, cells in culture, one or more laboratory test animals, or one or more human subjects. Methods to select and test control samples are within the ability of those in the art. Determination of statistical significance is within the ability of those skilled in the art, e.g., the number of standard deviations from the mean that constitute a positive result.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation.

“Differentially expressed” or “different level” as used herein refers to either an increased or decreased level relative to a control sample or value. For example, in some embodiments an increased level (of biomarker expression or other indicator or measurement) can be indicative of the reduced viability of a cell in response to a drug or other perturbation, and a decreased or equal level can be indicative of no change in the viability of a cell. In other embodiments, an equal or increased level (of biomarker expression or other indicator or measurement) can be indicative of the reduced viability of a cell in response to a drug or other perturbation, and a decreased level can be indicative of no change in the viability of a cell. In additional embodiments, a decreased level (of biomarker expression or other indicator or measurement) can be indicative of the reduced viability of a cell in response to a drug or other perturbation, and an increased or equal level can be indicative of no change in the viability of a cell. In still other embodiments, an equal or decreased level (of biomarker expression or other indicator or measurement) can be indicative of the reduced viability of a cell in response to a drug or other perturbation, and an increased level can be indicative of no change in the viability of a cell.

“Determining” as used herein includes qualitative and/or quantitative detection (i.e. detecting and/or measuring expression level) with or without reference to a control or a predetermined value.

As used herein, the term “neoplasia” refers to the formation or presence of a new, abnormal growth of tissue. The term “cancer” refers to a malignant neoplasm (Stedman's Medical Dictionary, 25th ed.; Hensyl ed.; Williams & Wilkins: Philadelphia, 1990). Exemplary cancers include, but are not limited to, pancreatic cancer (e.g., pancreatic andenocarcinoma, intraductal papillary mucinous neoplasm (IPMN), Islet cell tumors) and melanoma. Additional exemplary cancers include, but are not limited to, colorectal cancer (e.g., colon cancer, rectal cancer, colorectal adenocarcinoma), endometrial cancer (e.g., uterine cancer, uterine sarcoma), esophageal cancer (e.g., adenocarcinoma of the esophagus, Barrett's adenocarcinoma), and gastric cancer (e.g., stomach adenocarcinoma (STAD)), including, e.g., colon adenocarcinoma (COAD), oesophageal carcinoma (ESCA), ovarian cancer (e.g., cystadenocarcinoma, ovarian embryonal carcinoma, ovarian adenocarcinoma, clear cell ovarian cancer), rectal adenocarcinoma (READ) and uterine corpus endometrial carcinoma (UCEC). Other exemplary forms of cancer include, but are not limited to, diffuse large B-cell lymphoma (DLBCL), as well as the broader class of lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; hematopoietic cancers (e.g., myeloid malignancies (e.g., acute myeloid leukemia (AML) (e.g., B-cell AML, T-cell AML), myelodysplastic syndrome, myeloproliferative neoplasm, chronic myelomonocytic leukemia (CMML) and chronic myelogenous leukemia (CML) (e.g., B-cell CML, T-cell CML)) and lymphocytic leukemia such as acute lymphocytic leukemia (ALL) (e.g., B-cell ALL, T-cell ALL) and chronic lymphocytic leukemia (CLL) (e.g., B-cell CLL, T-cell CLL)); brain cancer (e.g., meningioma, glioblastomas, glioma (e.g., astrocytoma, oligodendroglioma), medulloblastoma); lung cancer (e.g., bronchogenic carcinoma, small cell lung cancer (SCLC), non-small cell lung cancer (NSCLC), adenocarcinoma of the lung); acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); bronchus cancer; carcinoid tumor; cervical cancer (e.g., cervical adenocarcinoma); choriocarcinoma; chordoma; craniopharyngioma; connective tissue cancer; epithelial carcinoma; ependymoma; endotheliosarcoma (e.g., Kaposi's sarcoma, multiple idiopathic hemorrhagic sarcoma); Ewing's sarcoma; ocular cancer (e.g., intraocular melanoma, retinoblastoma); familiar hypereosinophilia; gall bladder cancer; gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); papillary adenocarcinoma; penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

As used herein, the term “next-generation sequencing” or “NGS” can refer to sequencing technologies that have the capacity to sequence polynucleotides at speeds that were unprecedented using conventional sequencing methods (e.g., standard Sanger or Maxam-Gilbert sequencing methods). These unprecedented speeds are achieved by performing and reading out thousands to millions of sequencing reactions in parallel. NGS sequencing platforms include, but are not limited to, the following: Massively Parallel Signature Sequencing (Lynx Therapeutics); 454 pyro-sequencing (454 Life Sciences/Roche Diagnostics); solid-phase, reversible dye-terminator sequencing (Solexa/Illumina); SOLiD technology (Applied Biosystems); Ion semiconductor sequencing (ion Torrent); and DNA nanoball sequencing (Complete Genomics). Descriptions of certain NGS platforms can be found in the following: Shendure, et al., “Next-generation DNA sequencing,” Nature, 2008, vol. 26, No. 10, 135-1 145; Mardis, “The impact of next-generation sequencing technology on genetics,” Trends in Genetics, 2007, vol. 24, No. 3, pp. 133-141; Su et al., “Next-generation sequencing and its applications in molecular diagnostics” Expert Rev Mol Diagn, 2011, 11 (3):333-43; and Zhang et al., “The impact of next-generation sequencing on genomics”, J Genet Genomics, 201, 38(3): 95-109.

As used herein, the phrase “test drug-responsive transcriptional signature” refers to a synthetic compilation of transcript prevalence and/or expression data (optionally analyzed, normalized or otherwise adapted for use as a reference) that is derived from a population of cells (optionally a population of a specific cell type, optionally one that is known to be responsive to test drug), employed as a reference for distinguishing between cells that are responsive to a test drug and those that are not.

The term “single cell transcriptional profile” refers to an assemblage of transcript prevalence and/or expression data (optionally analyzed, normalized or otherwise adapted for use in comparisons as described herein) obtained from a single cell (optionally, a cell that has been treated with a test drug). In embodiments, a single cell transcriptional profile of the instant disclosure can refer to transcript prevalence and/or expression data for as few as one transcript (e.g., where a single transcript can be used to classify a biopsy cell as test drug-responsive or test drug-nonresponsive), or for a number of transcripts that in certain embodiments can be used to classify a biopsy cell as test drug-responsive or test drug-nonresponsive.

As used herein, the term “subject” includes humans and mammals (e.g., mice, rats, pigs, cats, dogs, and horses). In many embodiments, subjects are mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be, for example, rodents (e.g., mice, rats, hamsters), rabbits, primates, or swine such as inbred pigs and the like.

As used herein, the terms “treatment,” “treating,” “treat” and the like, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease or condition in a mammal, particularly in a human, and includes: (a) preventing the disease from occurring in a subject which can be predisposed to the disease but has not yet been diagnosed as having it; (b) inhibiting the disease, i.e., arresting its development; and (c) relieving the disease, i.e., causing regression of the disease.

Unless specifically stated or obvious from context, as used herein, the term “or” is understood to be inclusive. Unless specifically stated or obvious from context, as used herein, the terms “a”, “an”, and “the” are understood to be singular or plural.

The phrase “pharmaceutically acceptable carrier” is art recognized and includes a pharmaceutically acceptable material, composition or vehicle, suitable for administering compounds of the present disclosure to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

A “therapeutically effective amount” of an agent described herein is an amount sufficient to provide a therapeutic benefit in the treatment of a condition or to delay or minimize one or more symptoms associated with the condition. A therapeutically effective amount of an agent means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Other features and advantages of the disclosure will be apparent from the following description of the preferred embodiments thereof, and from the claims. Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All published foreign patents and patent applications cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

The following detailed description, given by way of example, but not intended to limit the disclosure solely to the specific embodiments described, may best be understood in conjunction with the accompanying drawings, in which:

FIGS. 1A to 1G depict multiplexed transcriptional profiling across pools of cell lines. FIG. 1A shows a schematic diagram illustrating the MIX-Seq platform. FIG. 1B shows a heatmap of the likelihoods assigned by the SNP-classification model of each cell coming from its parental cell line. The model consistently picked out a single cell line, from among the 24 ‘in-pool’ cell lines, with high confidence. FIG. 1C shows a UMAP representation of cells treated with DMSO control (gray/light) or nutlin (red/dark) across a pool of 24 cell lines. Arrows indicate the shift in the population median coordinates for each cell line. The RCC10RGB population is circled as an example of a sensitive cell line. FIG. 1D shows a heatmap of the average log fold-change estimates for each cell line for top differentially expressed genes. Nutlin sensitivity is given by (1−area under dose response curve) (AUC, see Example 1 below). The RCC10RGB population corresponding to the plot in FIG. 1C is indicated as an example of a sensitive cell line. FIG. 1E shows a volcano plot of the strong gene expression changes observed in response to nutlin treatment across TP53 WT cell lines (n=7 experiments). Effect size estimates and p-values (not corrected for multiple comparisons) for this and subsequent differential expression analyses were estimated using the Limma-trend pipeline (Law et al. 2014 and Ritchie et al. 2015, see also Example 1). Vertical dotted lines indicate a log FC threshold of 1. FIG. 1F shows, in contrast to FIG. 1E above, a volcano plot of the weak gene expression changes observed in response to nutlin treatment (n=17) in TP53 mutant cell lines. FIG. 1G shows a gene set analysis that identified gene sets up-regulated (right) and down-regulated (left) by nutlin treatment in the TP53 WT cell lines.

FIGS. 2A to 2C depict viability-related and viability-independent transcriptional response components. FIG. 2A shows a UMAP representation of single-cell expression profiles in a 99 cell line pool treated with vehicle control (gray/light) or trametinib (red/dark). Arrows indicate trametinib-induced shift of population median coordinates for each cell line. FIG. 2B shows a histogram of the number of cells recovered in each cell line and condition. FIG. 2C, at left, shows a volcano plot of the viability-independent response for each gene, representing the ‘y-intercept’ of a linear fit of expression change to drug sensitivity (see Example 1). The inset at left shows this relationship for an example gene: EGR1. The blue line shows the linear regression trend line (with the 95% confidence interval (CI) shown in shaded gray). The inset below shows the top up- (right, red) and down-regulated (left, blue) gene sets. At right, a volcano plot is shown of the viability-related response for each gene, representing the ‘y-intercept’ of a linear fit of expression change to drug sensitivity (see Example 1). The inset at left shows this relationship for an example gene: EGR1. The blue line shows the linear regression trend line (with the 95% confidence interval (CI) shown in shaded gray). The inset below shows the top up- (right, red) and down-regulated (left, blue) gene sets.

FIGS. 3A to 3E demonstrate machine learning analysis powered by large-scale transcriptional profiling. FIG. 3A demonstrates the accuracy of models trained to predict sensitivity for each individual drug, comparing measured transcriptional responses and baseline ‘omics’ features using the same set of cell lines. Predictions based on transcriptional profiling at 6 and 24 hours post-treatment are indicated by the gold and blue dots respectively. FIG. 3B shows a plot of the Pearson correlation between each gene's transcriptional response (24 hours post-treatment) and drug sensitivity (1−AUC) across cell lines and drugs, compared with the gene's feature importance for the random forest predictive models. FIG. 3C shows a heatmap matrix of measured transcriptional responses across cell lines 24 hours after trametinib treatment. FIG. 3D, at top, shows the Eigenvalue spectrum of PCA applied to the matrix in FIG. 3C; and at lower left, the projection of each cell line's response onto PC1 is plotted against its measured trametinib sensitivity (yellow points indicate cell lines with activating RAS/RAF mutations). The linear regression trend line (with the shaded gray region indicating the 95% CI) is shown in blue. At lower right, a comparison of PC2 scores with expression of the melonoma-specific transcription factor SOX10 across cell lines is shown. FIG. 3E shows a UMAP embedding of transcriptional response profiles across drugs, cell lines, and post-treatment time points. Points are colored by treatment condition (drug and time point). The inset below shows a zoomed view of the region indicated by the rectangle, with the larger green dots representing responses of BRAF mutant melanoma lines to the BRAF inhibitor dabrafenib.

FIGS. 4A to 4E show the population heterogeneity identified in pre-perturbation and post-perturbation transcriptional programs. FIG. 4A shows a UMAP representation of gene expression profiles of DMSO (light gray outline) and nutlin-treated (dark red outline) cell populations for a pool of 24 cell lines (as in FIG. 1 above). Cells are colored by their inferred cell cycle phase. TP53 WT cell lines (red arrows) show a predominance of G0/G1-phase cells after nutlin treatment not observed in TP53 mutant cell lines (black arrows). FIG. 4B shows quantification of the change in proportion of cells in G0/G1 in each cell line (y-axis) in response to nutlin treatment elicits G1-arrest selectively among the TP53 WT cell lines. Error bars show the 95% CI for estimates of the change in G0/G1 cell proportions. FIG. 4C shows the average change in the fraction of cells in each cell cycle phase for each drug treatment (averages were weighted by measured drug sensitivity, all for 24 hour time points). FIG. 4D shows a UMAP plot demonstrating the emergence of two sub-population clusters of RCC10RGB cells, 24 hours after treatment with bortezomib. Dot color depicts inferred cell cycle phase and dot size represents treatment condition. FIG. 4E shows a heatmap of log-fold changes (LFC) of the top differentially expressed genes between the two sub-population clusters of bortezomib-treated cells relative to untreated control cells for each of the 9/24 cell lines that showed this bimodal response pattern. In nearly all cases, one cluster (labeled cluster 2) was characterized by a predominance of S-phase cells, while the other cluster (cluster 1) contained cells mostly in G0/G1 phase. Dotted lines indicate the two cluster sub-populations of RCC10RGB cells shown in FIG. 4D. FIG. 4F shows a volcano plot of differentially expressed genes in cluster 2 vs cluster 1, averaged across the 10 cell lines, as depicted in FIG. 4E. Genes identified as part of the S-phase signature are highlighted in red.

FIGS. 5A to 5G show dual-multiplexed transcriptional profiling across cell lines and time points. FIG. 5A shows a schematic diagram of an experiment using Cell Hashing to multiplex the scRNA-seq of cell line pools sampled at different time points following drug treatment. FIG. 5B shows a UMAP plot of 13,713 cells across a pool of 24 cell lines at different times following treatment with trametinib (shades of blue), or DMSO control (pink). FIG. 5C shows single-cell expression levels of EGR1 at different time points following trametinib treatment for examples of insensitive (left) and sensitive cell lines (right). Red dots depict the mean expression levels at each time point, and error bars show the interval+/−s.e.m. FIG. 5D shows single-cell expression levels of MCMI at different time points following trametinib treatment for examples of insensitive (left) and sensitive cell lines (right). Red dots depict the mean expression levels at each time point, and error bars show the interval+/−s.e.m. FIG. 5E, top, shows the time course of the viability-independent response observed for the top down-regulated genes. At bottom, the enrichment of HALLMARK_KRAS_SIGNALING_UP genes in the down-regulated viability-independent response at each time point is shown. FIG. 5F, top, shows the time course of the viability-related response for the top down-regulated genes. At bottom, the enrichment of HALLMARK_G2M_CHECKPOINT genes in the viability-related response at each time post-treatment is shown. FIG. 5G shows the average time course of G0/G1 cell cycle-arrest across cell lines (n=24 cell lines). Error bars indicate interval+/−s.e.m.

FIGS. 6A to 6E show SNP-based detection of doublets and low-quality cells. FIG. 6A shows the scatterplot of parameters from the SNP-based models (see Example 1) from an example experiment, showing classification of cells as singlets, doublets, and low-quality′. The y-axis shows the improvement of the doublet model fit over the singlet model, while the x-axis shows the best model goodness-of-fit. FIG. 6B shows the distribution of cells classified in each category across experiments. FIG. 6C shows the proportion of cells classified as doublets in each experiment (excluding low-quality cells), as a function of the number of cells recovered in the experiment. Red line shows the trend-line from depicting the expected relationship of doublet probability with cell density. The black line shows the linear regression trend line (with the gray shaded region showing the 95% CI interval). Green and blue points show doublet proportions estimated using the Demuxlet method (Kang et al. 2018) for experiments with smaller pools, with and without exclusion of low-quality cells, based on the analysis pipeline, respectively. FIG. 6D shows the use of the Demuxlet doublet detection as ‘ground truth’ compared to the false-negative and false-positive rates for the doublet classification procedure used in the instant disclosure for each experiment. Overall, Demuxlet tended to produce a somewhat higher rate of detected doublets, though both methods tended to call doublets at a higher rate than expected based on the cell loading density (FIG. 6C). FIG. 6E shows an example experiment in which the proportion of cells were estimated where the most likely doublet pair of reference cell lines were both among the ‘in-pool’ cell lines (24/494 possible cell lines). 89% of cells classified as doublets had both identified reference cell lines among those in the experimental pool (n=107 cells). For cells classified as singlets, there were only ˜6% (approximately chance level) (n=2417 cells).

FIGS. 7A and 7B show the agreement between SNP-based and gene expression (GE)-based cell classification. FIG. 7A shows a UMAP representation of expression profiles from an example negative-control experiment (combination of DMSO-treated and untreated cells) in a pool of 24 cell lines. Cells show clear clustering by cell line (color indicates SNP-based parental cell line classification). Black dots show cells classified as doublets. Red dots indicate cells where gene expression and SNP-based classifications disagreed (0.2% of single cells, 16/6926). FIG. 7B shows a UMAP representation of expression profiles from an example (DMSO-treated) dataset from a 99 cell-line pool. Cells show clear clustering by cell line (color indicates SNP-based parental cell line classification). Black dots show cells classified as doublets. Red dots indicate cells where gene expression and SNP-based classifications disagreed (only 0.05% of single cells).

FIG. 8 shows the dependence of SNP-based cell classification on number of detected SNPs. At top is shown the error rate of cell classification as estimated based on the fraction of cells (n=2,417 cells) classified among those in the experimental pool (24/494 reference cell lines from this example dataset). Classification accuracy for cells with fewer SNP sites detected was estimated by randomly down-sampling the single-cell SNP reads. At bottom is shown the distribution of the number of SNP sites detected for the cells (n=2,417 cells) measured in the example experiment.

FIGS. 9A-9C show the identification of compound MOA from transcriptional response profiles. FIG. 9A, at left, shows a volcano plot of the (across-cell-line) average transcriptional response to bortezomib (6 hours post-treatment). Differential expression analyses were performed using the Limma-trend pipeline (Law et al. 2014 and Ritchie et al. 2015). At right is shown the top gene sets enriched among the most strongly up-regulated and down-regulated genes. FIG. 9B, at left, shows a volcano plot of the (across-cell-line) average transcriptional response to gemcitabine (24 hours post-treatment). Differential expression analyses were performed using the Limma-trend pipeline. At right is shown the top gene sets enriched among the most strongly up-regulated and down-regulated genes. FIG. 9C, at left, shows a volcano plot of the (across-cell-line) average transcriptional response to everolimus (24 hours post-treatment). Differential expression analyses were performed using the Limma-trend pipeline. At right is shown the top gene sets enriched among the most strongly up-regulated and down-regulated genes.

FIG. 10 shows the agreement between MIX-Seq and L1000 transcriptional response profiles. Shown is a comparison of the average response profiles measured using MIX-Seq and the L1000 platform (Subramanian et al. 2017) for six different compounds. Blue dots represent the 978 ‘landmark’ genes measured directly in the L1000 platform, while the red dots represent computationally inferred genes in the L1000 dataset. In each case responses are averaged across available cell lines. L1000 data were also averaged across available doses, and time points to simplify comparisons (see Example 1). For the MIX-Seq data, bortezomib and trametinib responses were measured at 24 hours post-treatment. Five of the six drugs showed good agreement in the average response profiles. Agreement was poor for navitoclax, though notably a robust transcriptional response to navitoclax was not observed in the MIX-Seq data. Pearson correlation coefficients, and associated p-values, are reported in each plot for the landmark genes and all genes in blue and red, respectively.

FIGS. 11A to 11E show an example of a pooling method of transcriptional profiling of genetic perturbation. FIG. 11A shows the distribution of expression levels of GPX4 in an example cell line for cells infected with two sgRNAs targeting GPX4, vs. two control sgRNAs (sgLACZ: non-targeting control, sgOR2J2: ‘cutting control’), which demonstrated robust on-target knockdown of GPX4 expression. FIG. 11B shows a comparison of average GPX4 expression across a pool of 50 cell lines following GPX4 KO, compared with control, which demonstrated consistent on-target KD across cell lines. Red dot shows the example cell line from FIG. 11A. FIG. 11C shows a volcano plot of average transcriptional response to GPX4 KO across all cell lines. The top up-regulated gene EEF1A2 has been shown to play a role in regulating lipid metabolism (Jeganathan et al. 2007), consistent with the role of GPX4 in lipid metabolism. FIG. 11D shows that the gene set analysis of the average GPX4 KO response demonstrated up-regulation of N-glycan synthesis, which has been reported to regulate glutathione levels (Calle et al. 2000). FIG. 11E shows a volcano plot comparing the response to GPX4 KO in GPX4-dependent (n=18) and non-GPX4-dependent (n=15) cell lines (see Example 1). No genes were found to be significantly differentially expressed between the groups (at an FDR threshold of 0.1).

FIG. 12 shows the impact of cell population size on estimates of transcriptional response profiles. To understand how estimates of each cell line's transcriptional response depended on the number of cells sampled per treatment condition, a down-sampling analysis was performed. Specifically, for measured trametinib response (24 hours post-treatment, as in FIGS. 2A-2C above), the set of cell lines which had at least 100 cells sampled in each condition (n=45) were selected, and the data were restricted to a random set of 100 cells per condition. The LFC transcriptional response profile of each cell line was estimated using random subsets of cells. These were compared to subsampled estimates with the profiles derived from the starting set of 100 cells per condition. Profile similarity was assessed by the Pearson correlation of LFC vectors across the 5000 most variably-expressed genes, averaged across five repetitions of the down-sampling procedure for each cell line. Each line represents data from a different cell line, colored by the cell line's measured trametinib sensitivity (1−AUC).

FIGS. 13A and 13B show the similarity of viability-related transcriptional responses across drugs. FIG. 13A, at top, shows a heatmap of correlation between transcriptional response and drug sensitivity across cell lines for the 1000 genes with the strongest average correlation for the 8 selective drugs. (Middle) Heatmap of average log FC drug response across cell lines for 4 pan-toxic drugs tested showing similarity to the viability-related response profiles above. At bottom is shown a depiction of gene membership in three MSigDB gene sets (cell cycle=HALLMARK_G2M_CHECKPOINT; translation=REACTOME_TRANSLATION, and P53 signaling=HALLMARK_P53_PATHWAY). FIG. 13B shows a matrix of correlations for the transcriptional response profiles shown in FIG. 13A across all pairs of compounds. Viability-related responses of selective compounds, and average responses for pan-toxic compounds, were broadly similar across these 1000 genes (with the exception of navitoclax and AZD5591).

FIGS. 14A to 14G show the effect of cell line sample size on estimation of transcriptional response components. Analyses of the trametinib response components (as in FIGS. 2A-2C above; n=99 cell lines) were repeated with random subsampling of the cell lines. FIG. 14A shows the average correlation between the estimated response profile (log FC) when using a subsample of cell lines vs all the cell lines for the average response. FIG. 14B shows the average correlation between the estimated response profile (log FC) when using a subsample of cell lines vs all the cell lines for the viability-related components. FIG. 14C shows the average correlation between the estimated response profile (log FC) when using a subsample of cell lines vs all the cell lines for the viability-independent components. Error bars in FIGS. 14A to 14C are s.e.m., while vertical red lines indicate the subsample sizes shown in FIGS. 14D to 14G (n=5, 10, 20, and 40 cell lines, respectively). FIG. 14D shows scatterplot comparisons of example estimates of average and viability-related response components using all cell lines vs random subsets of 5. FIG. 14E shows scatterplot comparisons of example estimates of average and viability-related response components using all cell lines vs random subsets of 10. FIG. 14F shows scatterplot comparisons of example estimates of average and viability-related response components using all cell lines vs random subsets of 20. FIG. 14G shows scatterplot comparisons of example estimates of average and viability-related response components using all cell lines vs random subsets of 40.

FIG. 15 shows a comparison of predictive models using transcriptional responses vs. using baseline features. FIG. 15 shows data similar to that of FIG. 3B, in which a plot of the Pearson correlation between each gene's transcriptional response (24 hours post-treatment) and drug sensitivity (1−AUC) across cell lines and drugs was compared with the gene's feature importance for the random forest predictive models. However, the model for the data presented here trained on baseline features data from all available cell lines, rather than using only the cell lines with measured transcriptional response data (as in FIG. 3B above).

FIGS. 16A and 16B show that the top principal component of observed transcriptional responses was well correlated with the variation in drug sensitivity. FIG. 16A shows the Pearson correlation for each cell line's measured drug sensitivity and the projection of its transcriptional response profile onto the first principal component (PC), computed for each treatment (drug and post-treatment time point). FIG. 16B shows the Pearson correlation for each cell line's measured drug sensitivity and the projection of its transcriptional response profile onto the second principal component, computed for each treatment (drug and post-treatment time point). Significant correlations (FDR<0.1) are shown in red. For a majority of the experiments, drug sensitivity was well captured by the first PC. For afatinib, the second PC captured the variation in drug sensitivity.

FIGS. 17A and 17B show that principal component analysis (PCA) identified multiple components underlying variability in trametinib response. FIG. 17A shows the projection of each cell line's response onto PCI is plotted against its measured trametinib sensitivity (BRAF and KRAS mutants are shown in red and green, respectively). The blue line shows the linear regression trend line (with the 95% CI interval shown with the shaded gray). FIG. 17B shows a comparison of PC2 scores with expression of the melonoma-specific transcription factor SOX10 across cell lines (melanoma cell lines indicated with grey outlines).

FIG. 18 shows that drug-induced changes in cell cycle phase were well correlated with measured viability effects across cell lines. A comparison of the Pearson correlation between measured drug sensitivity and the changes in G2/M-, G0/G1-, and S-phase cell percentages for all compounds is shown. The lattice shows the regression plane of the z-coordinate.

FIG. 19 shows a direct measurement of relative drug sensitivity via scRNA-seq. Shown are correlation, and associated p-values, between existing drug sensitivity data (Iorio et al. 2016; Corsello et al. 2020) and the measured change in relative cell abundance (based on pooled scRNA-seq data). The time point of scRNA-seq profiling is indicated by dot color.

FIGS. 20A and 20B show that single-cell RNA-seq allowed analysis of perturbation responses in heterogeneous populations. As an example demonstrating the ability of MIX-Seq to resolve differential transcriptional responses among distinct sub-populations of cells, two distinct sub-populations of IALM cells (present in the baseline data) were identified, and the transcriptional response of each sub-population to trametinib was measured. FIG. 20A shows a UMAP plot of the observed response of each sub-population to trametinib treatment (24 hours post-treatment). Fill color depicts inferred cell-cycle phase, and dot size depicts treatment condition. FIG. 20B shows a comparison of the average trametinib response for cells from the two IALM clusters shown in FIG. 20A. Dot color represents the significance of the difference between trametinib responses of the two sub-populations. Differential expression analysis was performed using the edgeR quasi-likelihood approach (Lun et al. 2016) as used in Soneson et al. 2018.

FIGS. 21A and 21B show that Cell Hashing provided efficient labeling of cells from different experimental conditions. FIG. 20A shows a t-SNE representation of hashtag read profiles across single cells from all cell lines, colored by the classified treatment condition. Read count profiles across hashtags were normalized for each cell using the centered log-ratio transformation (with a pseudocount value of 1 prior to computing the t-SNE embedding. Gray dots indicate cells classified as doublets. FIG. 21B shows a histogram of cell counts by parental cell line and the inferred hashtag condition.

FIG. 22 demonstrates a lack of time-dependent transcriptional response observed following treatment with DMSO vehicle control. Volcano plots show the average (across cell lines) transcriptional response to each treatment condition, using untreated cells as reference. No genes showed significant changes (FDR<0.1) in any DMSO-treated conditions, and there were no time-dependent trends apparent in the DMSO response. As a result, DMSO conditions and untreated cells were combined as reference populations for other analyses.

FIG. 23 demonstrates that estimation of viability-independent and viability-related response components was unbiased by cell count. To ensure that analyses of the viability-independent and viability-related time-courses of trametinib responses in FIGS. 5E and 5F above were unbiased by the number of cells available in each cell line and post-treatment time points, these response components were recomputed after averaging the normalized expression profiles across cells for each cell line, rather than summing the read counts across cells as in FIGS. 5E and 5F above. Averaging the normalized expression profiles ensured that the results were unbiased by the number of available cells. Comparison of the viability-independent (top) and viability-related (bottom) trametinib response components at each post-treatment time point showed close agreement independent of the method of aggregating data across cells. Pearson correlation coefficients and associated p-values are included for each plot.

FIGS. 24A and 24B show the variability of dabrafenib responses among BRAF mutant melanomas. FIG. 24A shows a scatterplot comparing the average response of IGR1 and A375 cells to dabrafenib treatment (24 hour post-treatment) across genes. Genes with significantly different responses observed between the two cell lines are indicated in red. FIG. 24B shows a heatmap of dabrafenib responses across six BRAF mutant melanoma lines for the top 50 genes with the most variable responses. LFC estimates were computed with a pseudo-count parameter of 10 to stabilize estimates for low-abundance genes.

FIG. 25 shows the top 100 genes most predictive of a cell's drug response according to the models employed in the instant disclosure. The Pearson correlation between each gene's transcriptional response (24 hours post-treatment) and drug sensitivity (1−AUC) across cell lines and drugs is shown, as well as the gene's relative importance in the random forest predictive models.

FIG. 26 shows the experimental and analysis details of perturbations used in the transcriptional response assays, including the timepoints assayed, the number of cell lines, the number of cell barcode labels added, the number of single cells, the 10× reagent chemistry used on the suspended cell, the cellranger version used in the analysis, and whether the assay was superloaded (superloaded samples were loaded at 1,500 cells per microliter, with up to 40,000 cells loaded per 10× channel for superloaded samples, with expected recovery of up to 20,000 cells per channel).

FIG. 27 shows the cell lines used in the instant disclosure, including the CCLE (cancer cell line encyclopedia) identification name, the Depmap ID, and the experiments in which each of the cell lines was used.

### DETAILED DESCRIPTION OF THE INVENTION

The current disclosure relates, at least in part, to the discovery of methods for performing reliable and rapid drug-response profiling of a biopsy sample of a subject, via use of single cell transcriptional profiling. Such predictive drug-response profiling can be performed with robust accuracy even upon a heterogeneous population of biopsy cells.

Accurate prediction of therapeutic responses in a clinically useful manner requires implementation of rapid ex vivo functional profiling of tumor cell sensitivity in combination with genomic profiling. The instant disclosure describes a novel approach to functional therapeutic response prediction that employs single-cell RNA-sequencing to derive post-treatment transcription profiles, which are compared with known drug-responsive transcriptional signatures, to determine treatment response.

Significantly, the methods and compositions of the current disclosure do not require propagation of biopsy-derived cell lines and/or growth of organoids derived from biopsy cells to predict drug-responsiveness of a biopsied sample with robust levels of accuracy approaching those obtained by more delayed methods involving, e.g., propagation of organoids and/or cell lines prior to testing of drug-responsiveness. Rather, in certain aspects, the instant disclosure obtains one or more single cell transcriptional profiles from biopsied cells and then compares such single cell transcriptional profiles with reference drug-responsive transcriptional signatures. In certain embodiments, such reference drug-responsive transcriptional signatures are assembled from cell line and/or organoid expression data, where drug-responsive outcomes have been extensively tracked/are known. Optionally, such reference drug-responsive transcriptional signatures have been assembled in a high-throughput manner from drug-response testing of mixed populations of cell lines, e.g., where the identity of responsive cells within a tested population of cells is discerned via SNP genotyping of such cells and such SNP genotypes are correlated with drug-responsive transcriptional signatures.

While high-throughput transcriptional profiling of drug-responsive states has been previously described in the art (see, e.g., Ye et al. Nature Communications 9: 4307 and Michalski et al. Brit. J. of Cancer 99: 760-767), the instant disclosure provides a process that allows for robust prediction of drug-responsive cells at a very early stage post-biopsy, as the instant process employs as inputs single cell transcriptome data for comparison with reference transcriptome signatures generated by massively parallel assessment of known cancer cell lines and/or organoids.

Development of rapid turnaround assays that accurately predict treatment response has been a major goal in translational oncology research. Accurate prediction of therapeutic responses in a clinically useful manner is expected to rely upon implementation of rapid ex vivo functional profiling of tumor cell sensitivity in combination with genomic profiling. The instant disclosure provides a novel approach to functional therapeutic response prediction using single-cell RNA-sequencing to derive post-treatment signatures that correlate with treatment responses. The approach described herein has been termed “MIX-Seq” Multiplexed Interrogation of gene eXpression through single-cell RNA Sequencing. In certain embodiments, application of the MIX-Seq process in a clinical setting has also been termed “TREAT-Seq” (for Therapeutic Response Evaluation through Assessment of Transcriptome Sequencing) herein. As disclosed herein, thousands of transcriptional profiles have been analyzed across hundreds of experimental small molecule and genetic perturbations using the Connectivity Map database and novel post-treatment signatures have been established that can be obtained from in vitro treatment of cancer models with a range of chemotherapy and targeted therapy drugs. Both core signatures that show common features across drugs as well as specific signatures in defined classes of drugs have been identified. Using a multiplexed single-cell RNA-sequencing based profiling platform, novel gene expression signatures have been discovered that show very robust correlations with drug sensitivity in longer-term viability assays across a panel of drugs assayed in approximately 100 different cell lines. MIX-Seq therefore addresses several attributes needed for an early predictive assay, including the ability to profile treatment response on smaller numbers of cells in the presence of other cell types from the tumor microenvironment with widely available sequencing-based technology. These observations indicate that translating the currently disclosed approaches (for accurately measuring/identifying drug-responsive signatures in a small number of cells derived from needle biopsies) to clinical care will allow for dramatically improved treatment response prediction.

For the majority of cancer patients, there are no standard biomarker-driven strategies to date to guide the choice of treatment regimens. Empiric therapy with highly toxic regimens can lead to significant morbidity from cancer treatments while only a subset of patients achieve a therapeutic benefit. While a small subset of cancer patients have clinically relevant genomic lesions, the majority of patients do not harbor actionable alterations, and additional strategies for therapeutic selection are needed to guide treatments for these patients. The development of rapid turnaround assays that accurately predict treatment response is a major goal in translational oncology research. Accurate prediction of therapeutic responses in a clinically useful manner will require implementation of rapid ex vivo functional profiling of tumor cell sensitivity in combination with genomic profiling. A novel approach to functional therapeutic response prediction using single-cell RNA-sequencing to derive post-treatment signatures that correlate with treatment responses was developed. This approach is termed MIX-Seq: Multiplexed Interrogation of gene eXpression through single-cell RNA Sequencing. MIX-Seq addressed several necessities of an early predictive assay, including the ability to profile treatment response on smaller numbers of cells in the presence of other cell types from the tumor microenvironment with widely available sequencing-based technology.

Thousands of transcriptional profiles across hundreds of experimental small molecule and genetic perturbations using the Connectivity Map database and novel post-treatment signatures were established from in vitro treatment of cancer models using a range of chemotherapy and targeted therapy drugs. Core signatures that show common features across drugs as well as specific signatures in defined classes of drugs were identified. Using a multiplexed single-cell RNA-sequencing based profiling platform, novel gene expression signatures were discovered. These signatures showed robust correlations with drug sensitivity in longer-term viability assays performed using a panel of drugs in ˜100 different cell lines. These observations indicate that because this approach is translatable to clinical care, and accurately measures drug response signatures on a small number of cells derived from needle biopsies, this approach dramatically improves treatment response prediction.

Assays to study cancer cell responses to pharmacologic or genetic perturbations are typically restricted to using simple phenotypic readouts such as proliferation rate. Information-rich assays, such as gene-expression profiling, have generally not permitted efficient profiling of a given perturbation across multiple cellular contexts. The instant disclosure describes “MIX-seq” (Multiplexed Interrogation of gene eXpression through single-cell RNA Sequencing), a method for multiplexed transcriptional profiling of post-perturbation responses across a mixture of samples with single-cell resolution, using SNP-based computational demultiplexing of single-cell RNA-sequencing data. MIX-seq was used to profile responses to chemical or genetic perturbations across pools of 100 or more cancer cell lines. MIX-Seq was combined with Cell Hashing to further multiplex additional experimental conditions, such as post-treatment time points or drug doses. Analysis of the high-content readout of scRNA-seq (single-cell RNA sequencing) reveals both shared and context-specific transcriptional response components that identify drug mechanism-of-action (MOA) and enable prediction of long-term cell viability responses from short-term transcription responses to treatment.

Large-scale screens of chemical and genetic vulnerabilities across hundreds of cancer cell lines were used to identify new therapeutic targets, and provide key insights into cancer biology and gene function (Tsherniak et al. 2017; Meyers et al. 2017; McDonald et al. 2017; Iorio et al. 2016; Behan et al. 2019; Barretina et al. 2012; Garnett et al. 2012). The ability of these approaches to reveal the cellular mechanisms and pathways underlying such cancer vulnerabilities was limited by the reliance on a single readout of cell viability to assess the effects of each perturbation. In contrast, high-content readouts provided opportunities to capture a more detailed picture of the cellular effects of a perturbation that underlie observed fitness effects (Norman et al.) or that arose independently of any observable fitness effects. In particular, expression profiles emerged as a robust and informative phenotypic measure of cellular responses to perturbations, with applications such as identifying drug mechanism-of-action (MOA), gene function, and gene regulatory networks (Dixit et al. 2016; Adamson et al. 2016; Subramanian et al. 2017; Norman et al. 2019; Lamb et al. 2006; Subramanian et al. 2017). High-throughput gene expression profiling (e.g. Luminex-based in a limited number of contexts (Subramanian et al. 2017; Bush et al. 2017; Ye et al. 2018) was used to produce large datasets of perturbation signatures—most notably the Connectivity Map (CMAP, Subramanian et al. 2017)—enabling systematic analysis of the space of transcriptional responses across perturbations.

However, extant methods including those described above have required each perturbation or cell type to be profiled separately, which has limited their cost-effectiveness and broader adoption. In particular, previous efforts have largely focused on studying responses in a small number of cell line contexts. However, perturbation responses are often context specific, reflecting their interaction with the cell's underlying genomic or functional features. For example, targeted drugs elicited responses only in cell lines harboring particular oncogenic mutations, or expressing certain genes, making observed results specific to the particular cell line models chosen. More generally, the inability to efficiently measure transcriptional responses across cell contexts has limited the understanding of how perturbation effects differ across the broad range of genomic and molecular cell states, which could be critical for predicting the therapeutic response of patient tumors.

The advent of single-cell genomics (Klein et al. 2015 and Macosko et al. 2015) and development of methods for profiling cell viability in pooled cell cultures (Yu et al. 2016) has helped address these challenges. In parallel, assays such as Perturb-Seq (Dixit et al. 2016 and Adamson et al. 2016) combined pooled perturbation screens with a single-cell RNA-Seq (scRNA-seq) readout and may lead to the development of methods with the necessary scale and resolution to assay many cells within a mixed culture. A related study (Srivatsan et al. 2019) used massively parallel scRNA-seq with combinatorial barcoding to profile the responses of a few individual cell lines to diverse drugs and doses. However, in contrast to extant methods, the instant disclosure has now successfully profiled the responses of many diverse cell lines to a given perturbation, to assess context-specific effects.

To facilitate the study of post-treatment gene expression signatures across multiple cell lines in parallel, MIX-Seq: Multiplexed Interrogation of gene eXpression through single-cell RNA Sequencing was developed. This approach combined: i) the ability to pool hundreds of cancer cell lines and co-treat them with one or more perturbations, and ii) the power of single-cell RNA sequencing (scRNA-seq) to simultaneously profile the cells' responses and resolve the identity of each cell based on single nucleotide polymorphism (SNP) profiles. The instant disclosure demonstrates that MIX-Seq enabled efficient study of transcriptional signatures after pharmacologic or genetic perturbation, evaluation of temporal evolution of post-perturbation transcriptional response, investigation of the mechanism of action of novel small-molecule compounds, and the development of novel therapeutic response prediction methods in cancer cell models.

TREAT-seq as disclosed herein leverages a number of single-cell RNA-sequencing platforms, including Seq-Well, Drop-Seq and others. Other RNA profiling approaches can also be utilized (for example, Ye et al. Nat Commun. 9(1):4307 and Bush et al. Nat Commun. 8(1):105, each of which is incorporated herein by reference in its entirety).

The instant disclosure accordingly presents an experimental and computational platform (MIX-Seq) for performing highly multiplexed transcriptional profiling of perturbation responses across many cell contexts using single-cell RNA-seq applied to co-treated pools of cancer cell lines. The efficacy of this approach was demonstrated by profiling the responses of pools of 24-99 cell lines to a range of different drugs, as well as to CRISPR perturbations.

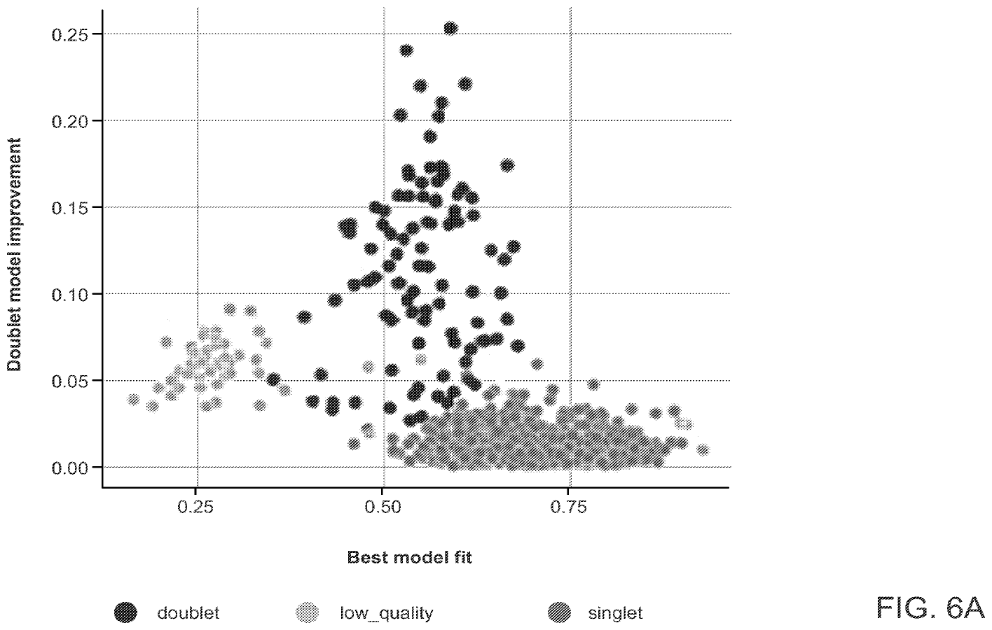

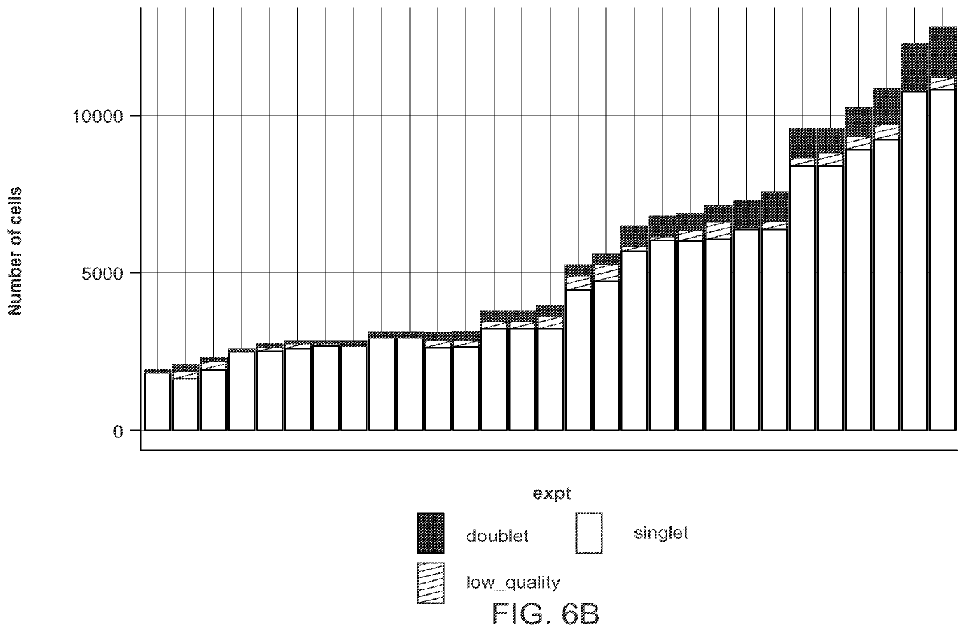

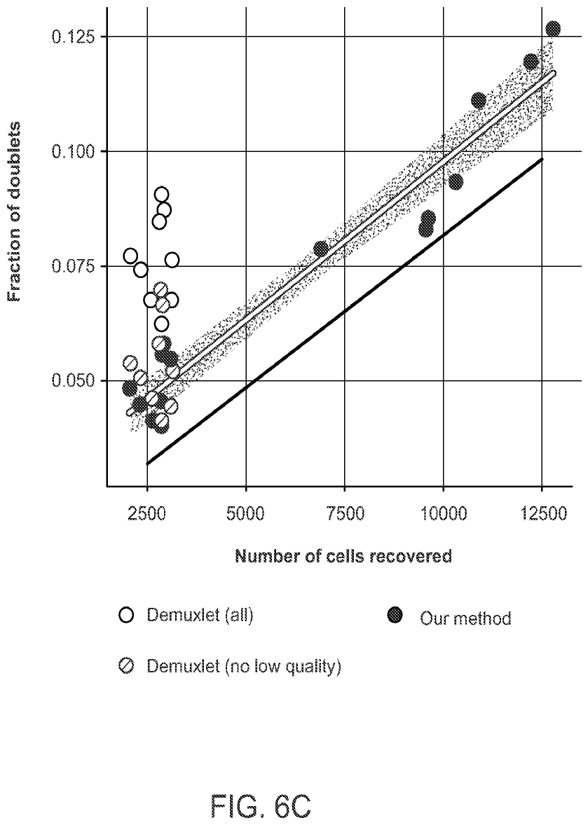

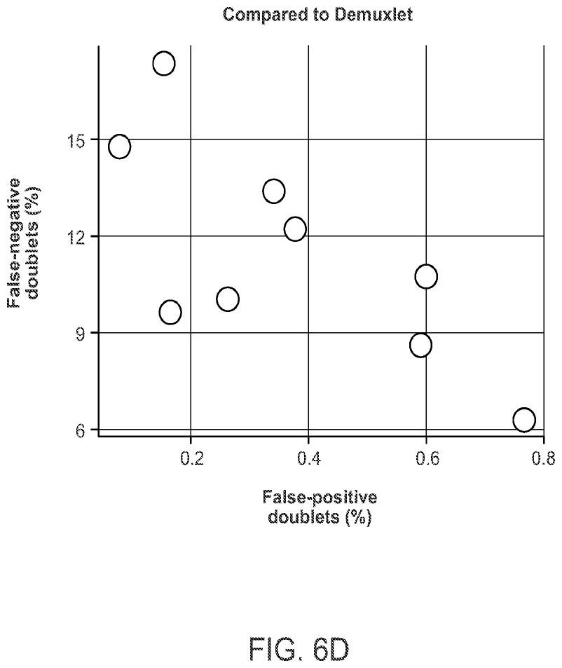

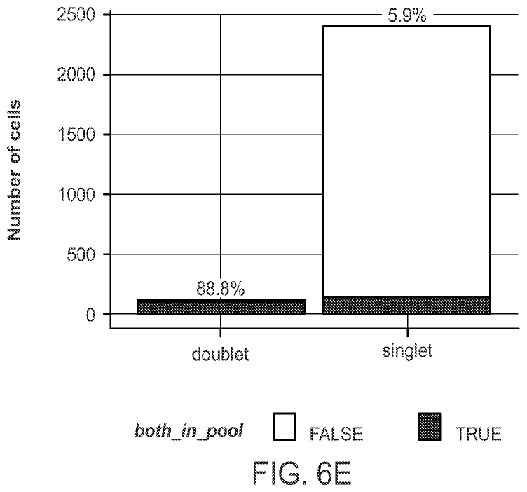

To determine the cell line identity of each cell, an optimized computational demultiplexing method was developed that utilized the unique SNP profiles of each cell, showing that it was able to classify single cells with negligible error rates, even at low sequencing depths. This approach also allowed for identification of droplets containing ambient mRNA (empty droplets) or two cells (doublets′). The instant method draws on other recently published SNP-demultiplexing methods (Huang et al. 2019 and Xu et al. 2019), most notably Demuxlet (Kang et al. 2018), which also used pre-computed reference SNP profiles. However, rather than detecting doublets by explicitly computing the likelihood of all possible reference mixtures, as in Demuxlet, the instant method utilized a fast approximation based on generalized linear models (see Example 1) that efficiently scaled to much larger cell line pools. For smaller pools where both models could be applied, the model was verified to produce identical single-cell classifications, and similar doublet detection results were compared to Demuxlet (FIGS. 6A to 6E).

A number of approaches were heretofore developed for high-throughput transcriptional profiling and have been applicable to study perturbation responses at scale. The Connectivity Map project utilized a low-cost bead-based assay that measures a reduced set of 1000 ‘landmark’ genes to profile thousands of different perturbation responses (Subramanian et al. 2017 and Lamb et al. 2003). Methods such as DRUG-seq (Ye et al. 2018) and PLATE-seq (Bush et al. 2017) used oligo-tagging of treatment conditions to perform multiplexed RNA-sequencing, greatly reducing library preparation costs. Similar sample-barcoding strategies were employed with scRNA-seq (Srivatsan et al. 2019, Stoeckius et al. 2018, and Shin et al. 2019), allowing for multiplexed profiling across treatment conditions such as time points and drugs. MIX-Seq complemented extant approaches by employing multiplexed profiling of perturbation responses across broad panels of heterogeneous cell contexts, without the need for additional experimental barcoding steps. As demonstrated herein, MIX-Seq was combined with existing sample-barcoding strategies, such as Cell Hashing, and enabled dual-multiplexing across treatment conditions and cell contexts.

The single-cell resolution of MIX-Seq also enabled a more detailed characterization of the perturbation responses of cancer cell populations. For example, the instant disclosure has demonstrated that analysis of changes in the inferred cell cycle composition across cell lines provided insights into the mechanisms underlying decreased proliferation. The instant disclosure also provides examples of how single-cell profiling was used to reveal heterogeneous responses within a sample, as well as to isolate differential responses among distinct subpopulations. Such capabilities are particularly important when studying more heterogenous samples, such as primary tumor samples, as well as for probing mechanisms of drug resistance.

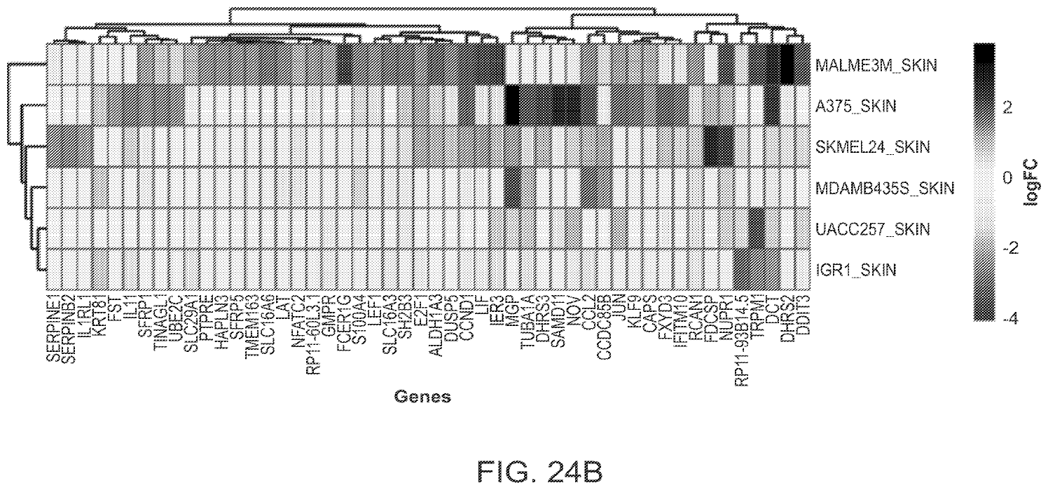

MIX-Seq's ability to efficiently profile genome-wide transcriptional responses across a broad panel of cell lines provided several advantages relative to traditional approaches. First, it allowed for the detection of context-specific responses, which were critical for assessing highly selective drugs like dabrafenib and nutlin. Even among sensitive cell lines, however, there was substantial response heterogeneity, and profiling many cell lines naturally made results less sensitive to the particular choice of cell line models under study. For example, it was observed that responses to the BRAF-inhibitor dabrafenib showed substantial variation, even among the highly sensitive BRAF-mutant melanoma cell lines (FIGS. 24A and 24B).

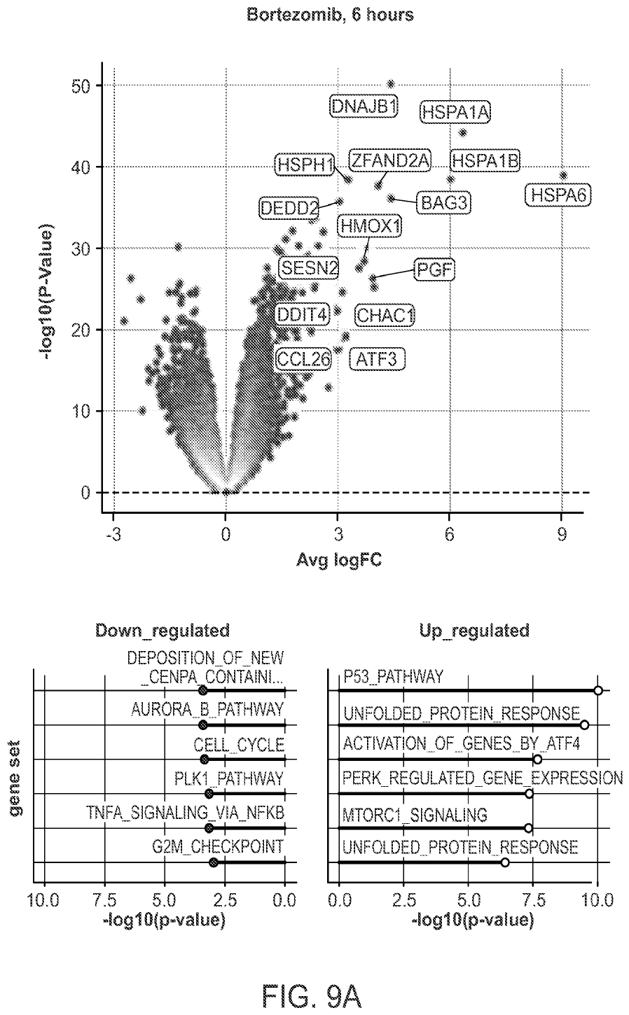

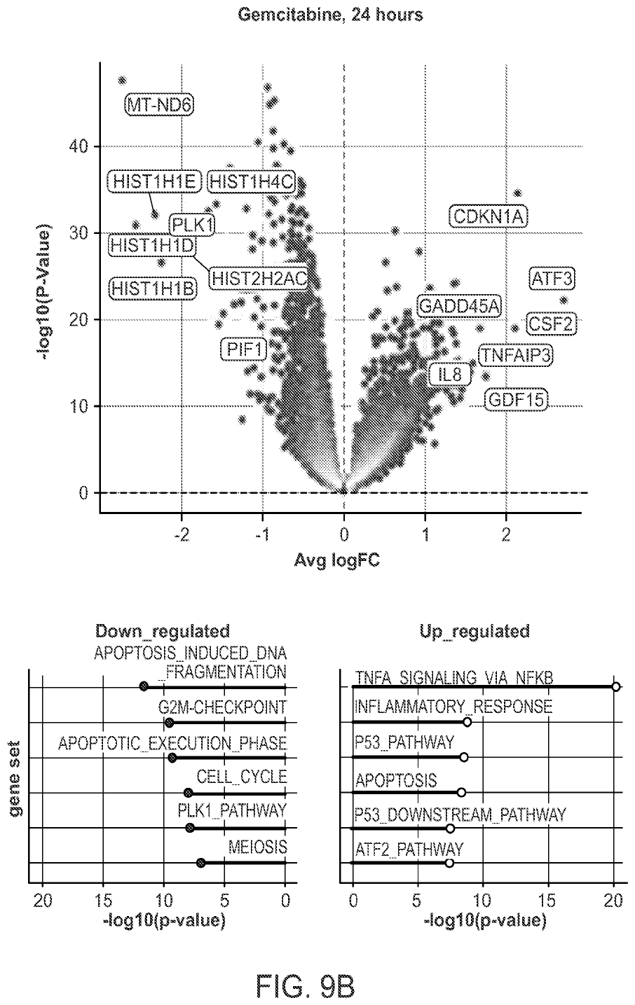

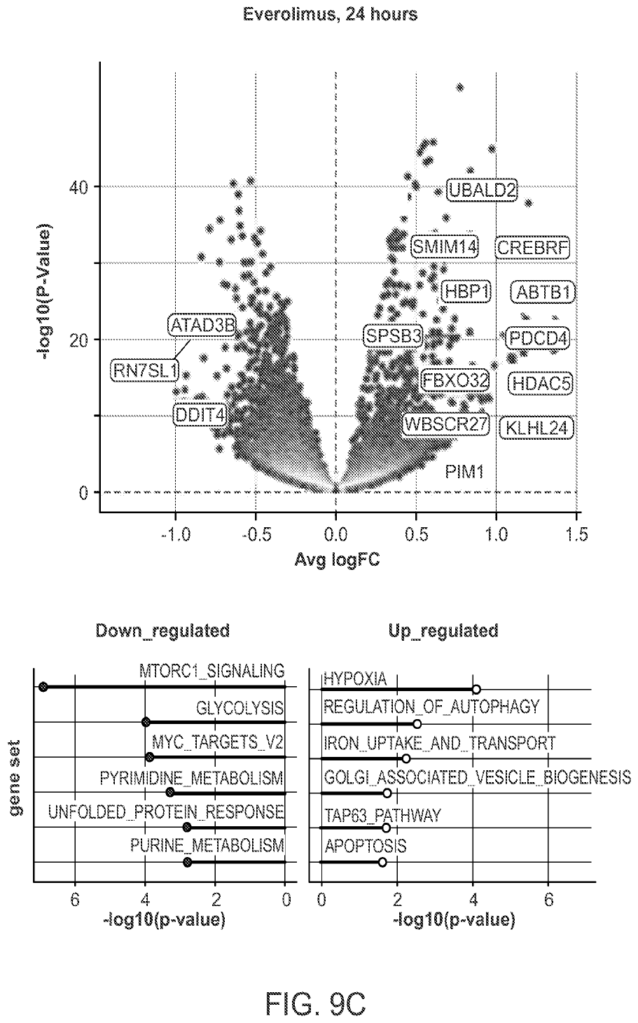

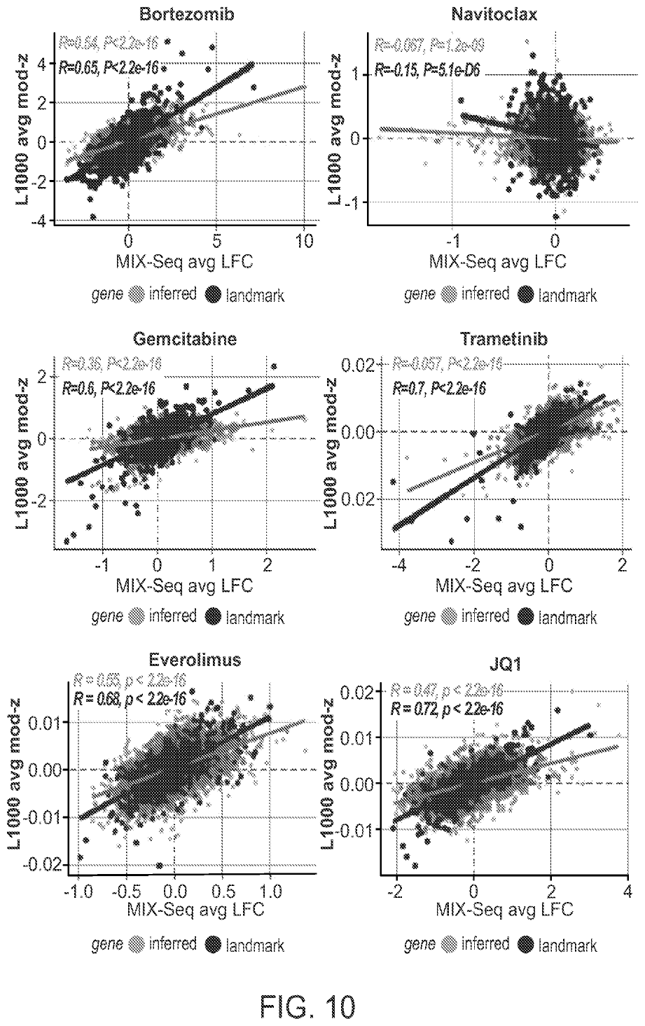

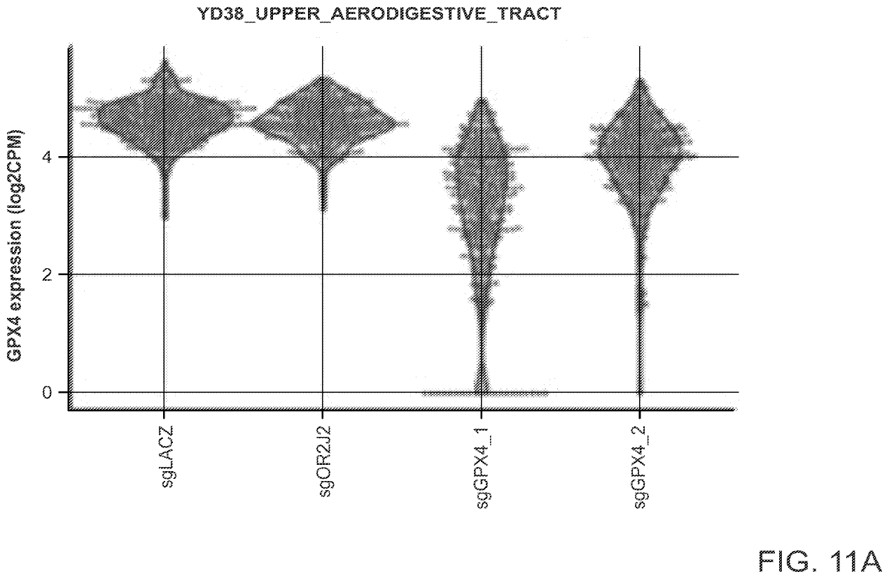

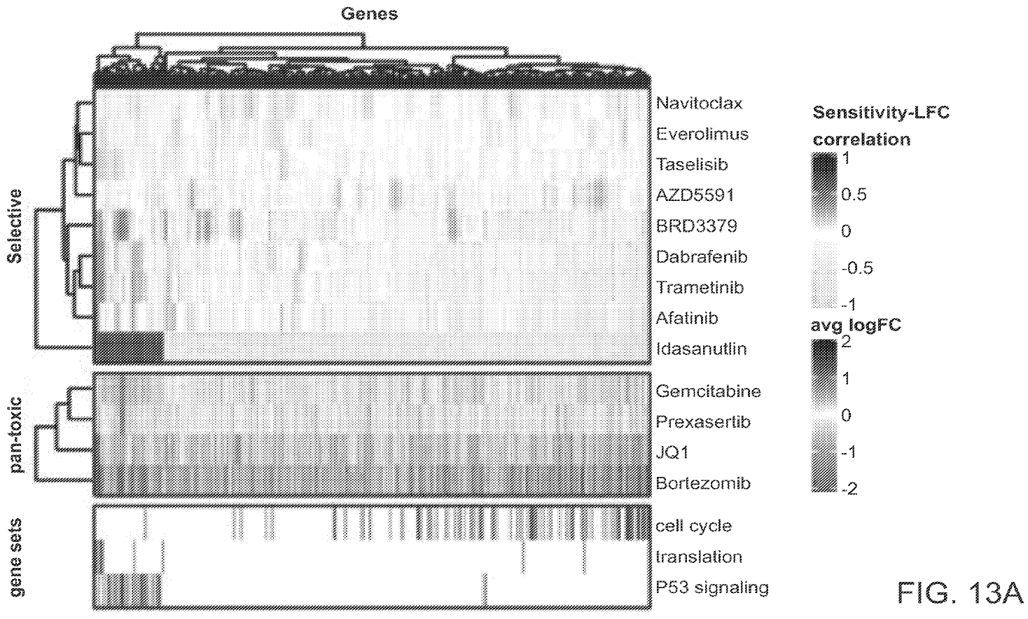

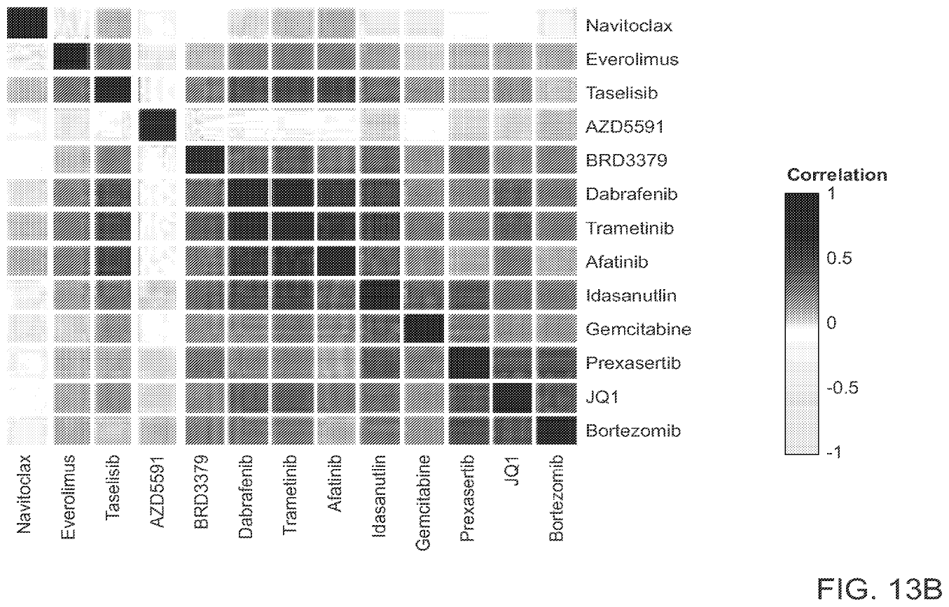

By profiling perturbation responses across large panels of well-characterized cell lines, patterns of how transcriptional changes relate to the underlying genomic and functional features of the cells were uncovered. In particular, pairing MIX-Seq with PRISM (Yu et al. 2016), which measures long-term drug sensitivity across the same panel of cell lines, allowed for the dissection of the transcriptional response components associated with decreased cell viability, and therefore elucidated the mechanisms underlying a drug's fitness effects. For the drugs studied here, viability-related responses were broadly similar across drugs, mostly reflecting a down-regulation of cell-cycle genes and up-regulation of genes involved in translation (FIG. 13A), though transcriptional signatures associated with apoptosis were also observed for some drugs (e.g., FIGS. 9A-9C). The two clear exceptions to this pattern were both inhibitors of anti-apoptotic proteins—the BCL-2 inhibitor navitoclax and the MCL1 inhibitor AZD5591. These drugs did not produce strong and/or selective transcriptional responses. This demonstrated that compounds which directly induced apoptosis did not elicit a clear transcriptional signature, at least when measured 24 hours post-treatment as provided herein.

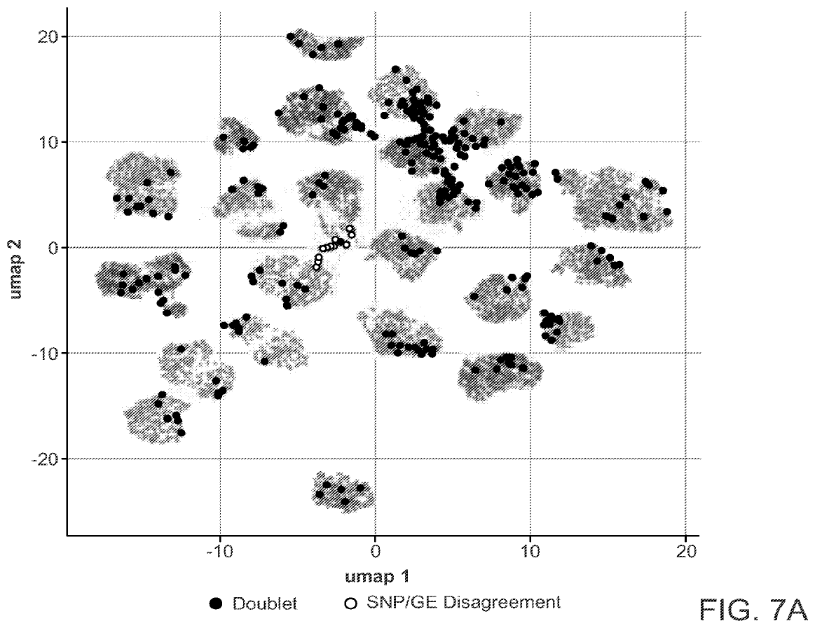

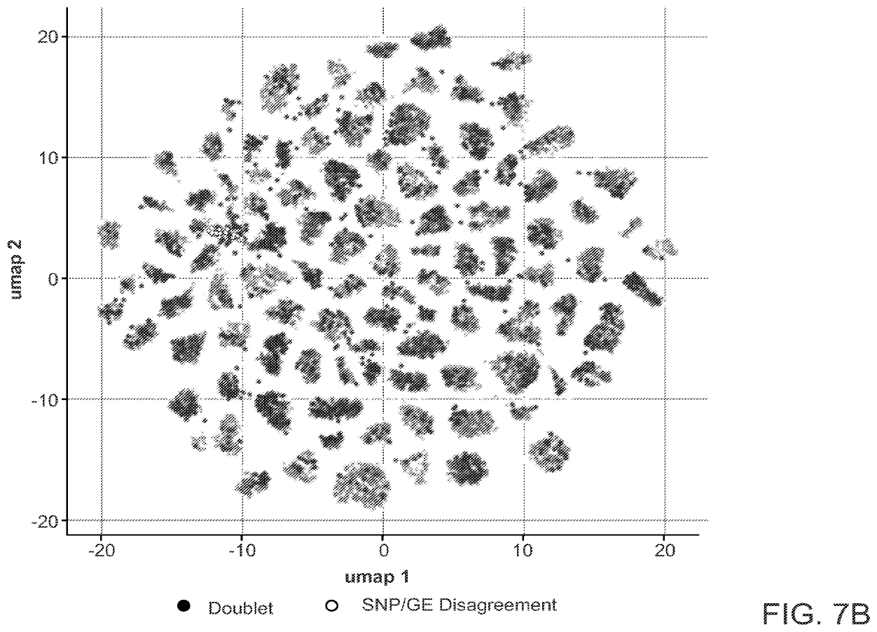

One potential caveat of profiling transcriptional responses in pools of cell lines is that paracrine signaling between cell lines in the pool affects the measured responses. However, it was observed that scRNA-seq profiles at baseline for cells grown in a pooled format were consistently most similar to bulk RNA-seq measurements of the same cell lines grown individually (FIGS. 7A and 7B), which indicated that such paracrine-signaling effects were likely modest. Measuring treatment and control conditions within the same pool of cell lines also provided some internal control for baseline effects of paracrine signaling. Finally, previous work showed that drug response profiles measured in cell line pools were largely concordant with standard measurements (Yu et al. 2016). Nevertheless, the potential for interactions between cell lines in the pool should also be considered when measuring perturbation responses using MIX-Seq.

MIX-Seq was also used to show that transcriptional responses measured 6-24 hours after drug treatment could predict long-term cell viability remarkably well across selected targeted cancer drugs. These results were in broad agreement with recently published analyses (Szalai et al. 2019 and Jones et al. 2020) that compared drug sensitivity data and transcriptional profiling data from the CMAP project (Subramanian et al. 2017 and Lamb et al. 2006), spanning many compounds in a core set of cell lines. By allowing efficient profiling of a given drug's transcriptional effects across many cell lines, the MIX-Seq approach of the instant disclosure offered unique opportunities to evaluate these relationships in detail for individual compounds, rather than requiring analyses that pool data across compounds, as in previous work. Notably, for the drugs tested here, the ability of machine learning models to predict drug sensitivity from a cell line's transcriptional response was substantially better than when using baseline omics features. These results indicated that transcriptional profiling provided a robust pharmacodynamic marker of drug sensitivity, which improved predictions of tumor vulnerabilities compared with standard biomarker approaches. An important further contemplated application of this approach is to utilize scRNA-seq to rapidly assess the sensitivity of primary tumor cells to various drug treatments ex vivo, circumventing the prolonged primary cell cultures needed to achieve sufficient cell numbers for standard long-term viability assays such as Cell-Titer-Glo (Kodack et al. 2017 and Tseng et al. 2019).

The instant disclosure therefore demonstrates that, by measuring perturbation responses across many different cell contexts, with single-cell resolution, MIX-Seq as disclosed herein has provided a powerful tool for identifying the core transcriptional programs of cancer cells, and an understanding of how perturbations interact with the underlying cell context to alter these programs.

Additional details of the methods of the instant disclosure are described in the following sections.

### Sequencing Methods

Certain methods and compositions provided herein employ methods of sequencing nucleic acids. A number of DNA (including cDNA) sequencing techniques are known in the art, including fluorescence-based sequencing methodologies (See, e.g., Birren et al., Genome Analysis Analyzing DNA, 1, Cold Spring Harbor, N.Y., which is incorporated herein by reference in its entirety). In some embodiments, automated sequencing techniques understood in that art are utilized. In some embodiments, parallel sequencing of partitioned amplicons can be utilized (PCT Publication No WO2006084132, which is incorporated herein by reference in its entirety). In some embodiments, DNA sequencing is achieved by parallel oligonucleotide extension (See, e.g., U.S. Pat. Nos. 5,750,341; 6,306,597, which are incorporated herein by reference in their entireties). Additional examples of sequencing techniques include the Church polony technology (Mitra et al, 2003, Analytical Biochemistry 320, 55-65; Shendure et al, 2005 Science 309, 1728-1732; U.S. Pat. Nos. 6,432,360, 6,485,944, 6,511,803, which are incorporated by reference), the 454 picotiter pyrosequencing technology (Margulies et al, 2005 Nature 437, 376-380; US 20050130173, which are incorporated herein by reference in their entireties), the Solexa single base addition technology (Bennett et al, 2005, Pharmacogenomics, 6, 373-382; U.S. Pat. Nos. 6,787,308; 6,833,246, which are incorporated herein by reference in their entireties), the Lynx massively parallel signature sequencing technology (Brenner et al. (2000). Nat. Biotechnol. 18:630-634; U.S. Pat. Nos. 5,695,934; 5,714,330, which are incorporated herein by reference in their entireties), and the Adessi PCR colony technology (Adessi et al. (2000). Nucleic Acid Res. 28, E87; WO 00/018957, which are incorporated herein by reference in their entireties).

Next-generation sequencing (NGS) methods can be employed in certain aspects of the instant disclosure to obtain a high volume of sequence information (such as are particularly required to perform deep sequencing of bead-associated RNAs following capture of RNAs from cryosections) in a highly efficient and cost-effective manner. NGS methods share the common feature of massively parallel, high-throughput strategies, with the goal of lower costs in comparison to older sequencing methods (see, e.g., Voelkerding et al., Clinical Chem., 55: 641-658, 2009; MacLean et al., Nature Rev. Microbiol, 7: 287-296; which are incorporated herein by reference in their entireties). NGS methods can be broadly divided into those that typically use template amplification and those that do not. Amplification-utilizing methods include pyrosequencing commercialized by Roche as the 454 technology platforms (e.g., GS 20 and GS FLX), the Solexa platform commercialized by Illumina, and the Supported Oligonucleotide Ligation and Detection (SOLiD™) platform commercialized by Applied Biosystems. Non-amplification approaches, also known as single-molecule sequencing, are exemplified by the HeliScope platform commercialized by Helicos Biosciences, SMRT sequencing commercialized by Pacific Biosciences, and emerging platforms marketed by VisiGen and Oxford Nanopore Technologies Ltd.

In pyrosequencing (U.S. Pat. Nos. 6,210,891; 6,258,568, which are incorporated herein by reference in their entireties), template DNA is fragmented, end-repaired, ligated to adaptors, and clonally amplified in-situ by capturing single template molecules with beads bearing oligonucleotides complementary to the adaptors. Each bead bearing a single template type is compartmentalized into a water-in-oil microvesicle, and the template is clonally amplified using a technique referred to as emulsion PCR. The emulsion is disrupted after amplification and beads are deposited into individual wells of a picotitre plate functioning as a flow cell during the sequencing reactions. Ordered, iterative introduction of each of the four dNTP reagents occurs in the flow cell in the presence of sequencing enzymes and luminescent reporter such as luciferase. In the event that an appropriate dNTP is added to the 3′ end of the sequencing primer, the resulting production of ATP causes a burst of luminescence within the well, which is recorded using a CCD camera. It is possible to achieve read lengths greater than or equal to 400 bases, and 10⁶ sequence reads can be achieved, resulting in up to 500 million base pairs (Mb) of sequence.

In the Solexa/Illumina platform (Voelkerding et al., Clinical Chem., 55: 641-658, 2009; MacLean et al., Nature Rev. Microbiol, 7: 287-296; U.S. Pat. Nos. 6,833,246; 7,115,400; 6,969,488, which are incorporated herein by reference in their entireties), sequencing data are produced in the form of shorter-length reads. In this method, single-stranded fragmented DNA is end-repaired to generate 5′-phosphorylated blunt ends, followed by Klenow-mediated addition of a single A base to the 3′ end of the fragments. A-addition facilitates addition of T-overhang adaptor oligonucleotides, which are subsequently used to capture the template-adaptor molecules on the surface of a flow cell that is studded with oligonucleotide anchors. The anchor is used as a PCR primer, but because of the length of the template and its proximity to other nearby anchor oligonucleotides, extension by PCR results in the “arching over” of the molecule to hybridize with an adjacent anchor oligonucleotide to form a bridge structure on the surface of the flow cell. These loops of DNA are denatured and cleaved. Forward strands are then sequenced with reversible dye terminators. The sequence of incorporated nucleotides is determined by detection of post-incorporation fluorescence, with each fluorophore and block removed prior to the next cycle of dNTP addition. Sequence read length ranges from 36 nucleotides to over 50 nucleotides, with overall output exceeding 1 billion nucleotide pairs per analytical run.

Sequencing nucleic acid molecules using SOLiD technology (Voelkerding et al, Clinical Chem., 55: 641-658, 2009; U.S. Pat. Nos. 5,912,148; and 6,130,073, which are incorporated herein by reference in their entireties) can initially involve fragmentation of the template, ligation to oligonucleotide adaptors, attachment to beads, and clonal amplification by emulsion PCR. Following this, beads bearing template are immobilized on a derivatized surface of a glass flow-cell, and a primer complementary to the adaptor oligonucleotide is annealed. However, rather than utilizing this primer for 3′ extension, it is instead used to provide a 5′ phosphate group for ligation to interrogation probes containing two probe-specific bases followed by 6 degenerate bases and one of four fluorescent labels. In the SOLiD system, interrogation probes have 16 possible combinations of the two bases at the 3′ end of each probe, and one of four fluors at the 5′ end. Fluor color, and thus identity of each probe, corresponds to specified color-space coding schemes. Multiple rounds (usually 7) of probe annealing, ligation, and fluor detection are followed by denaturation, and then a second round of sequencing using a primer that is offset by one base relative to the initial primer. In this manner, the template sequence can be computationally re-constructed, and template bases are interrogated twice, resulting in increased accuracy. Sequence read length averages 35 nucleotides, and overall output exceeds 4 billion bases per sequencing run.

In certain embodiments, nanopore sequencing is employed (see, e.g., Astier et al, J. Am. Chem. Soc. 2006 Feb. 8; 128(5): 1705-10, which is incorporated by reference). The theory behind nanopore sequencing has to do with what occurs when a nanopore is immersed in a conducting fluid and a potential (voltage) is applied across it. Under these conditions a slight electric current due to conduction of ions through the nanopore can be observed, and the amount of current is exceedingly sensitive to the size of the nanopore. As each base of a nucleic acid passes through the nanopore (or as individual nucleotides pass through the nanopore in the case of exonuclease-based techniques), this causes a change in the magnitude of the current through the nanopore that is distinct for each of the four bases, thereby allowing the sequence of the DNA molecule to be determined.

The Ion Torrent technology is a method of DNA sequencing based on the detection of hydrogen ions that are released during the polymerization of DNA (see, e.g., Science 327(5970): 1190 (2010); U.S. Pat. Appl. Pub. Nos. 20090026082, 20090127589, 20100301398, 20100197507, 20100188073, and 20100137143, which are incorporated herein by reference in their entireties). A microwell contains a template DNA strand to be sequenced. Beneath the layer of microwells is a hypersensitive ISFET ion sensor. All layers are contained within a CMOS semiconductor chip, similar to that used in the electronics industry. When a dNTP is incorporated into the growing complementary strand a hydrogen ion is released, which triggers a hypersensitive ion sensor. If homopolymer repeats are present in the template sequence, multiple dNTP molecules will be incorporated in a single cycle. This leads to a corresponding number of released hydrogens and a proportionally higher electronic signal. This technology differs from other sequencing technologies in that no modified nucleotides or optics are used. The per base accuracy of the Ion Torrent sequencer is approximately 99.6% for 50 base reads, with approximately 100 Mb generated per run. The read-length is 100 base pairs. The accuracy for homopolymer repeats of 5 repeats in length is approximately 98%. The benefits of ion semiconductor sequencing are rapid sequencing speed and low upfront and operating costs.

### Biopsy Samples

In some aspects of the instant disclosure, biopsy samples are obtained from a subject. It is contemplated that any art-recognized method for obtaining a sample from a subject can be employed, with exemplary biopsy procedures including, without limitation, needle biopsy (e.g., fine needle biopsy), CT-guided biopsy, ultrasound-guided biopsy, bone biopsy, bone marrow biopsy, liver biopsy, kidney biopsy, aspiration biopsy, prostate biopsy, skin biopsy and surgical biopsy, among others.

### Methods for Transcriptome/Expression Profiling

Certain aspects of the instant disclosure employ transcriptome/expression profiling, optionally upon single cells. While it is contemplated that most known methods for transcriptome/expression profiling can be employed for the processes disclosed herein, exemplary known methods for obtaining transcriptome data/expression profiling include the Seq-Well approach (Gierahn et al. Nature Methods 14: 395-398) and the Drop-seq approach to scRNA-seq (see, e.g., WO 2016/040476), among others.

### Chemotherapeutic Drugs

In certain aspects of the instant disclosure, chemotherapeutic drugs are employed (e.g., to contact biopsy samples/cells, cell lines, organoids, administered to a subject, etc.). Exemplary known chemotherapeutic drugs include, but are not limited to, bifunctional alkylators: cyclophosphamide, mechlorethamine, chlorambucil, melphalan; monofunctional alkylators:dacarbazine, nitrosoureas, temozolomide(oral dacarbazine); anthracyclines: daunorubicin, doxorubicin, epirubicin, idarubicin, mitoxantrone, valrubicin; cytoskeletal disruptors (taxanes): paclitaxel, docetaxel, abraxane. taxotere, epothilones; histone deacetylase inhibitors: vorinostat, romidepsin; inhibitors of topoisomerase i:irinotecan, topotecan; inhibitors of topoisomerase etoposide; teniposide, tafluposide; kinase inhibitors: bortezomib, erlotinib, gefitinib, imatinib, vemurafenib, vismodegib; nucleotide analogs and precursor analogs: azacitidine, azathioprine, capecitabine, cytarabine, doxifluridine, fluorouracil, gemcitabine, hydroxyurea, mercaptopurine, methotrexate, tioguanine; peptide antibiotics: bleomycin, actinomycin; platinum-based agents: carboplatin, cisplatin, oxaliplatin, retinoids, tretinoin, alitretinoin, bexarotene; and vinca alkaloids and derivatives: vinblastine, vincristine, vindesine, vinorelbine. In some embodiments, the chemotherapeutic drugs dabrafenib, trametinib, bortezomib and/or nutlin are specifically employed.

Dabrafenib

Dabrafenib is a FDA-approved drug for the treatment of cancers associated with a mutated version of the gene BRAF. Dabrafenib acts as an inhibitor of the associated enzyme B-Raf, which plays a role in the regulation of cell growth. Dabrafenib has exhibited clinical activity with a manageable safety profile in clinical trials of phase 1 and 2 in patients with BRAF (V600)-mutated metastatic melanoma. The structure of dabrafenib is:

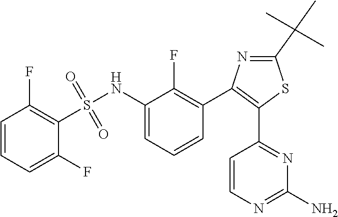 [SMILES: CC(C)(C)c1nc(-c2cccc(NS(=O)(=O)c3c(F)cccc3F)c2F)c(-c2ccnc(N)n2)s1]

An exemplary dosage of dabrafenib is 150 mg daily taken by tablet (50 mg and 75 mg tablets) until disease progression or unacceptable toxicity occurs.

Trametinib

Trametinib is a FDA-approved MEK inhibitor drug with anti-cancer activity. It inhibits MEK1 and MEK2. Trametinib has been commonly used to treat metastatic melanoma carrying the BRAF V600E mutation and it is routinely combined with the BRAF inhibitor dabrafenib. The structure of trametinib is:

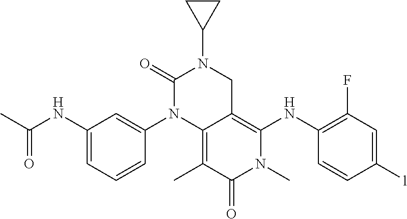

An exemplary dosage of trametinib is 2 mg daily by tablet (0.5 mg and 2 mg tablets; sometimes combined with 150 mg dabrafenib daily) until disease progression or unacceptable toxicity occurs.

Bortezomib

Bortezomib is a FDA-approved proteasome inhibitor commonly used for the treatment of relapsed multiple myeloma and mantle cell lymphoma. Bortezomib has also been shown to increase the sensitivity of cancer cells to traditional anticancer agents (e.g., gemcitabine, cisplatin, and paclitaxel). The structure of bortezomib is:

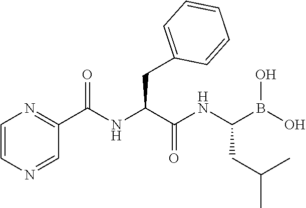 [SMILES: CC(C)C[C@H](NC(=O)[C@H](Cc1ccccc1)NC(=O)c1cnccn1)B(O)O]

An exemplary dosage of bortezomib is 1.3 mg/m² given intravenously for a 2 week period followed by a 10 day rest period for six 3-week cycles (can be extended up to eight 3-week cycles).

Nutlin

Nutlins are cis-imidazoline analogs which inhibit the interaction between mdm2 and tumor suppressor p53. Nutlin-3 is the compound most commonly used in anti-cancer studies, particularly for liposarcoma (clinical trial phase I). Nutlin small molecules occupy the p53 binding pocket of MDM2 and effectively disrupt the p53-MDM2 interaction that leads to activation of the p53 pathway in p53 wild-type cells. Inhibiting the interaction between mdm2 and p53 stabilizes p53, and is believed to selectively induce senescence in cancer cells. Several derivatives of nutlin, such as RG7112 and RG7388 (Idasanutlin) have been developed and progressed into human studies The structure of nutlin (nutlin 3) is:

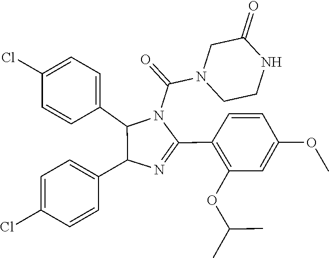 [SMILES: COc1ccc(C2=NC(c3ccc(Cl)cc3)C(c3ccc(Cl)cc3)N2C(=O)N2CCNC(=O)C2)c(OC(C)C)c1]

An exemplary dosage of nutlin (RG7112; nutlin analog in a phase I clinical trial) is 1440 mg/m² (IV) daily for 10 days on a 28 day cycle followed by surgical removal of tumor.

### Other Drugs

Certain aspects of the instant disclosure have employed a number of other compounds, including navitoclax, everolimus, CGS15943, AZD5591, afatinib, JQ1, gemcitabine, taselisib and prexasertib.

Navitoclax is a Bcl-2 inhibitor having the following structure:

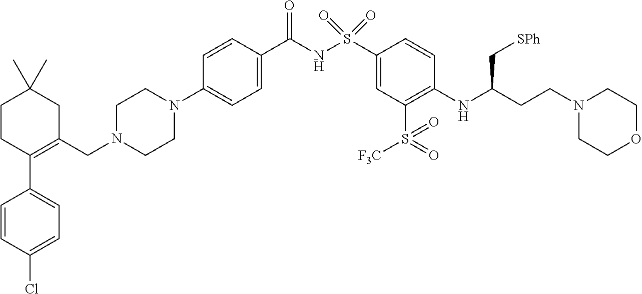

Everolimus is a mTOR inhibitor used as an immunosuppressant to prevent rejection of organ transplants and in the treatment of renal cell cancer and other tumours, having the following structure:

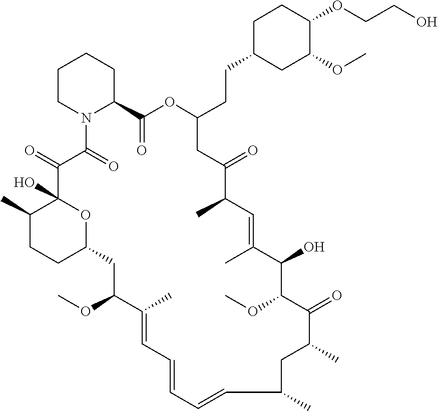

CGS15943 is a potent and reasonably selective antagonist for the adenosine receptors A1 and A2A having the following structure:

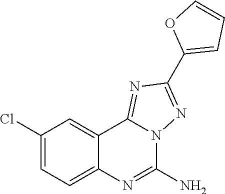 [SMILES: Nc1nc2ccc(Cl)cc2c2nc(-c3ccco3)nn12]

AZD5591 is a MCL-1-targeting BH3-mimetic.

Afatinib is a tyrosine kinase inhibitor used for treatment of non-small cell lung carcinoma (NSCLC), having the following structure:

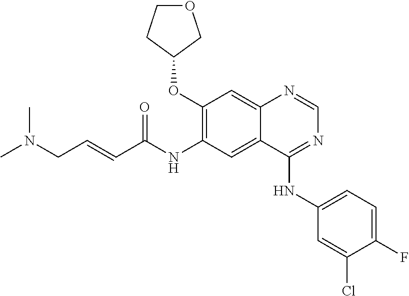 [SMILES: CN(C)C/C=C/C(=O)Nc1cc2c(Nc3ccc(F)c(Cl)c3)ncnc2cc1O[C@@H]1CCOC1]

JQ1 is a thienotriazolodiazepine and a potent inhibitor of the BET family of bromodomain proteins which include BRD2, BRD3, BRD4, and the testis-specific protein BRDT in mammals. BET inhibitors structurally similar to JQ1 are being tested in clinical trials for a variety of cancers including NUT midline carcinoma. JQ1 has the following structure:

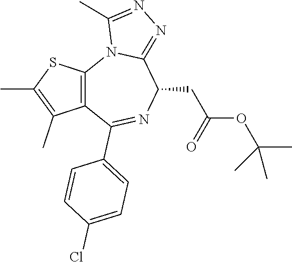 [SMILES: Cc1sc2c(c1C)C(c1ccc(Cl)cc1)=N[C@@H](CC(=O)OC(C)(C)C)c1nnc(C)n1-2]

Gemcitabine is a is a synthetic pyrimidine nucleoside prodrug—a nucleoside analog in which the hydrogen atoms on the 2′ carbon of deoxycytidine are replaced by fluorine atoms, used to treat a number of types of cancers, including breast cancer, ovarian cancer, non-small cell lung cancer, pancreatic cancer, and bladder cancer. Gemcitabine has the following structure:

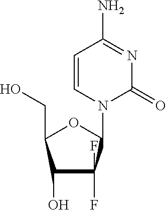

Taselisib is a small molecule inhibitor targeting the p110α protein product of the phosphoinositide 3-kinase gene, PIK3CA, having the following structure:

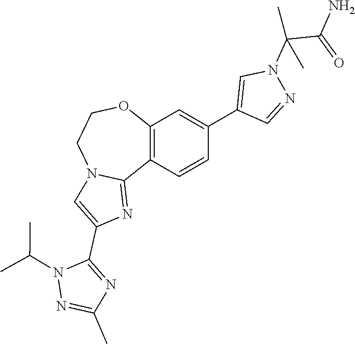 [SMILES: Cc1nc(-c2cn3c(n2)-c2ccc(-c4cnn(C(C)(C)C(N)=O)c4)cc2OCC3)n(C(C)C)n1]

Prexasertib is a small molecule checkpoint kinase inhibitor, mainly active against CHEK1, with minor activity against CHEK2, which causes induction of DNA double-strand breaks resulting in apoptosis. Prexasertib has the following structure:

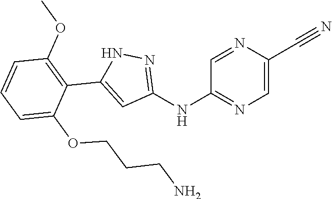 [SMILES: COc1cccc(OCCCN)c1-c1cc(Nc2cnc(C#N)cn2)n[nH]1]

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue, to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of a high value target gene inhibitor which achieves a half-maximal inhibition of symptoms) as determined in cell culture. Such information can be used to more accurately determine useful doses in humans. Levels in plasma may be measured, for example, by high performance liquid chromatography.

### Pharmaceutical Compositions

Agents of the present disclosure can be incorporated into a variety of formulations for therapeutic use (e.g., by administration) or in the manufacture of a medicament, by combining the agent(s) with appropriate pharmaceutically acceptable carriers or diluents, and may be formulated into preparations in solid, semi-solid, liquid or gaseous forms. Examples of such formulations include, without limitation, tablets, capsules, powders, granules, ointments, solutions, suppositories, injections, inhalants, gels, microspheres, and aerosols.

Pharmaceutical compositions can include, depending on the formulation desired, pharmaceutically-acceptable, non-toxic carriers of diluents, which are vehicles commonly used to formulate pharmaceutical compositions for animal or human administration. The diluent is selected so as not to affect the biological activity of the combination. Examples of such diluents include, without limitation, distilled water, buffered water, physiological saline, PBS, Ringer's solution, dextrose solution, and Hank's solution. A pharmaceutical composition or formulation of the present disclosure can further include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

Further examples of formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249: 1527-1533 (1990).

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink.

Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for parenteral administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of amines, carboxylic acids, and other types of compounds, are well known in the art. For example, S. M. Berge, et al. describe pharmaceutically acceptable salts in detail in J Pharmaceutical Sciences 66 (1977):1-19, incorporated herein by reference. The salts can be prepared in situ during the final isolation and purification of the compounds of the application, or separately by reacting a free base or free acid function with a suitable reagent, as described generally below. For example, a free base function can be reacted with a suitable acid. Furthermore, where the compounds to be administered of the application carry an acidic moiety, suitable pharmaceutically acceptable salts thereof may, include metal salts such as alkali metal salts, e.g. sodium or potassium salts; and alkaline earth metal salts, e.g. calcium or magnesium salts. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

Formulations may be optimized for retention and stabilization in a subject and/or tissue of a subject, e.g., to prevent rapid clearance of a formulation by the subject. Stabilization techniques include cross-linking, multimerizing, or linking to groups such as polyethylene glycol, polyacrylamide, neutral protein carriers, etc. to achieve an increase in molecular weight.

Other strategies for increasing retention include the entrapment of the agent, such as dabrafenib, trametinib, bortezomib, nutlin or other drug, in a biodegradable or bioerodible implant. The rate of release of the therapeutically active agent is controlled by the rate of transport through the polymeric matrix, and the biodegradation of the implant. The transport of drug through the polymer barrier will also be affected by compound solubility, polymer hydrophilicity, extent of polymer cross-linking, expansion of the polymer upon water absorption so as to make the polymer barrier more permeable to the drug, geometry of the implant, and the like. The implants are of dimensions commensurate with the size and shape of the region selected as the site of implantation. Implants may be particles, sheets, patches, plaques, fibers, microcapsules and the like and may be of any size or shape compatible with the selected site of insertion.

The implants may be monolithic, i.e. having the active agent homogenously distributed through the polymeric matrix, or encapsulated, where a reservoir of active agent is encapsulated by the polymeric matrix. The selection of the polymeric composition to be employed will vary with the site of administration, the desired period of treatment, patient tolerance, the nature of the disease to be treated and the like. Characteristics of the polymers will include biodegradability at the site of implantation, compatibility with the agent of interest, ease of encapsulation, a half-life in the physiological environment.

Biodegradable polymeric compositions which may be employed may be organic esters or ethers, which when degraded result in physiologically acceptable degradation products, including the monomers. Anhydrides, amides, orthoesters or the like, by themselves or in combination with other monomers, may find use. The polymers will be condensation polymers. The polymers may be cross-linked or non-cross-linked. Of particular interest are polymers of hydroxyaliphatic carboxylic acids, either homo- or copolymers, and polysaccharides. Included among the polyesters of interest are polymers of D-lactic acid, L-lactic acid, racemic lactic acid, glycolic acid, polycaprolactone, and combinations thereof. By employing the L-lactate or D-lactate, a slowly biodegrading polymer is achieved, while degradation is substantially enhanced with the racemate. Copolymers of glycolic and lactic acid are of particular interest, where the rate of biodegradation is controlled by the ratio of glycolic to lactic acid. The most rapidly degraded copolymer has roughly equal amounts of glycolic and lactic acid, where either homopolymer is more resistant to degradation. The ratio of glycolic acid to lactic acid will also affect the brittleness of in the implant, where a more flexible implant is desirable for larger geometries. Among the polysaccharides of interest are calcium alginate, and functionalized celluloses, particularly carboxymethylcellulose esters characterized by being water insoluble, a molecular weight of about 5 kD to 500 kD, etc. Biodegradable hydrogels may also be employed in the implants of the individual instant disclosure. Hydrogels are typically a copolymer material, characterized by the ability to imbibe a liquid. Exemplary biodegradable hydrogels which may be employed are described in Heller in: Hydrogels in Medicine and Pharmacy, N. A. Peppes ed., Vol. III, CRC Press, Boca Raton, Fla., 1987, pp 137-149.

### Pharmaceutical Dosages

Pharmaceutical compositions of the present disclosure containing an agent described herein may be used in accord with known methods, such as oral administration, intravenous administration as a bolus or by continuous infusion over a period of time, by intramuscular, intraperitoneal, intracerobrospinal, intracranial, intraspinal, subcutaneous, intraarticular, intrasynovial, intrathecal, topical, or inhalation routes.

Dosages and desired drug concentration of pharmaceutical compositions of the present disclosure may vary depending on the particular use envisioned. The determination of the appropriate dosage or route of administration is well within the skill of an ordinary artisan. Animal experiments provide reliable guidance for the determination of effective doses for human therapy. Interspecies scaling of effective doses can be performed following the principles described in Mordenti, J. and Chappell, W. “The Use of Interspecies Scaling in Toxicokinetics,” In Toxicokinetics and New Drug Development, Yacobi et al., Eds, Pergamon Press, New York 1989, pp. 42-46.

For in vivo administration of any of the agents of the present disclosure, normal dosage amounts may vary from about 10 ng/kg up to about 100 mg/kg of an individual's and/or subject's body weight or more per day, depending upon the route of administration. In some embodiments, the dose amount is about 1 mg/kg/day to 10 mg/kg/day. For repeated administrations over several days or longer, depending on the severity of the disease, disorder, or condition to be treated, the treatment is sustained until a desired suppression of symptoms is achieved.

An effective amount of an agent of the instant disclosure may vary, e.g., from about 0.001 mg/kg to about 1000 mg/kg or more in one or more dose administrations for one or several days (depending on the mode of administration). In certain embodiments, the effective amount per dose varies from about 0.001 mg/kg to about 1000 mg/kg, from about 0.01 mg/kg to about 750 mg/kg, from about 0.1 mg/kg to about 500 mg/kg, from about 1.0 mg/kg to about 250 mg/kg, and from about 10.0 mg/kg to about 150 mg/kg.

An exemplary dosing regimen may include administering an initial dose of an agent of the disclosure of about 200 μg/kg, followed by a weekly maintenance dose of about 100 μg/kg every other week. Other dosage regimens may be useful, depending on the pattern of pharmacokinetic decay that the physician wishes to achieve. For example, dosing an individual from one to twenty-one times a week is contemplated herein. In certain embodiments, dosing ranging from about 3 μg/kg to about 2 mg/kg (such as about 3 μg/kg, about 10 μg/kg, about 30 μg/kg, about 100 μg/kg, about 300 μg/kg, about 1 mg/kg, or about 2 mg/kg) may be used. In certain embodiments, dosing frequency is three times per day, twice per day, once per day, once every other day, once weekly, once every two weeks, once every four weeks, once every five weeks, once every six weeks, once every seven weeks, once every eight weeks, once every nine weeks, once every ten weeks, or once monthly, once every two months, once every three months, or longer. Progress of the therapy is easily monitored by conventional techniques and assays. The dosing regimen, including the agent(s) administered, can vary over time independently of the dose used.

Pharmaceutical compositions described herein can be prepared by any method known in the art of pharmacology. In general, such preparatory methods include the steps of bringing the agent or compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, Poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfate, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, Eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

To prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of an agent (e.g., dabrafenib, trametinib, bortezomib, nutlin or other drug) described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans, to render the compositions suitable for administration to various animals is well understood, and the ordinarily skilled veterinary pharmacologist can design and/or perform such modification with ordinary experimentation.

Drugs provided herein can be formulated in dosage unit form for ease of administration and uniformity of dosage. It will be understood, however, that the total daily usage of the agents described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The agents and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the agent or pharmaceutical composition described herein is suitable for topical administration to the eye of a subject.

The exact amount of an agent required to achieve an effective amount will vary from subject to subject, depending, for example, on species, age, and general condition of a subject, severity of the side effects or disorder, identity of the particular agent, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of an agent (e.g., a modulator of a high value target gene identified herein) described herein.

As noted elsewhere herein, a drug of the instant disclosure may be administered via a number of routes of administration, including but not limited to: subcutaneous, intravenous, intrathecal, intramuscular, intranasal, oral, transepidermal, parenteral, by inhalation, or intracerebroventricular.

The term “injection” or “injectable” as used herein refers to a bolus injection (administration of a discrete amount of an agent for raising its concentration in a bodily fluid), slow bolus injection over several minutes, or prolonged infusion, or several consecutive injections/infusions that are given at spaced apart intervals.

In some embodiments of the present disclosure, a formulation as herein defined is administered to the subject by bolus administration.

A drug or other therapy of the instant disclosure is administered to the subject in an amount sufficient to achieve a desired effect at a desired site, and/or in the subject as a whole, determined by a skilled clinician to be effective. In some embodiments of the disclosure, the agent is administered at least once a year. In other embodiments of the disclosure, the agent is administered at least once a day. In other embodiments of the disclosure, the agent is administered at least once a week. In some embodiments of the disclosure, the agent is administered at least once a month.

Additional exemplary doses for administration of an agent of the disclosure to a subject include, but are not limited to, the following: 1-20 mg/kg/day, 2-15 mg/kg/day, 5-12 mg/kg/day, 10 mg/kg/day, 1-500 mg/kg/day, 2-250 mg/kg/day, 5-150 mg/kg/day, 20-125 mg/kg/day, 50-120 mg/kg/day, 100 mg/kg/day, at least 10 μg/kg/day, at least 100 μg/kg/day, at least 250 μg/kg/day, at least 500 μg/kg/day, at least 1 mg/kg/day, at least 2 mg/kg/day, at least 5 mg/kg/day, at least 10 mg/kg/day, at least 20 mg/kg/day, at least 50 mg/kg/day, at least 75 mg/kg/day, at least 100 mg/kg/day, at least 200 mg/kg/day, at least 500 mg/kg/day, at least 1 g/kg/day, and a therapeutically effective dose that is less than 500 mg/kg/day, less than 200 mg/kg/day, less than 100 mg/kg/day, less than 50 mg/kg/day, less than 20 mg/kg/day, less than 10 mg/kg/day, less than 5 mg/kg/day, less than 2 mg/kg/day, less than 1 mg/kg/day, less than 500 μg/kg/day, and less than 500 μg/kg/day.

In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of an agent (e.g., dabrafenib, trametinib, bortezomib, nutlin or other drug) described herein. In certain embodiments, a dose described herein includes independently between 1 mg and 3 mg, inclusive, of an agent (e.g., dabrafenib, trametinib, bortezomib, nutlin or other drug) described herein. In certain embodiments, a dose described herein includes independently between 3 mg and 10 mg, inclusive, of an agent (e.g., dabrafenib, trametinib, bortezomib, nutlin or other drug) described herein. In certain embodiments, a dose described herein includes independently between 10 mg and 30 mg, inclusive, of an agent (e.g., dabrafenib, trametinib, bortezomib, nutlin or other drug) described herein. In certain embodiments, a dose described herein includes independently between 30 mg and 100 mg, inclusive, of an agent (e.g., dabrafenib, trametinib, bortezomib, nutlin or other drug) described herein.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult. In certain embodiments, a dose described herein is a dose to an adult human whose body weight is 70 kg.

It will be also appreciated that an agent (e.g., dabrafenib, trametinib, bortezomib, nutlin or other drug) or composition, as described herein, can be administered in combination with one or more additional pharmaceutical agents (e.g., therapeutically and/or prophylactically active agents), which are different from the agent or composition and may be useful as, e.g., combination therapies.

Combination therapies explicitly contemplated for the instant disclosure include, e.g., administration of first drug (e.g., dabrafenib, trametinib, bortezomib, nutlin or other drug) with a second drug, e.g., a different chemotherapeutic or other pharmaceutical agent.

The agent or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease described herein. Each additional pharmaceutical agent may be administered at a dose and/or on a time schedule determined for that pharmaceutical agent. The additional pharmaceutical agents may also be administered together with each other and/or with the agent or composition described herein in a single dose or administered separately in different doses. The particular combination to employ in a regimen will take into account compatibility of the agent described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

Dosages for a particular agent of the instant disclosure may be determined empirically in individuals who have been given one or more administrations of the agent.

Administration of an agent of the present disclosure can be continuous or intermittent, depending, for example, on the recipient's physiological condition, whether the purpose of the administration is therapeutic or prophylactic, and other factors known to skilled practitioners. The administration of an agent may be essentially continuous over a preselected period of time or may be in a series of spaced doses.

Guidance regarding particular dosages and methods of delivery is provided in the literature; see, for example, U.S. Pat. Nos. 4,657,760; 5,206,344; or 5,225,212. It is within the scope of the instant disclosure that different formulations will be effective for different treatments and different disorders, and that administration intended to treat a specific organ or tissue may necessitate delivery in a manner different from that to another organ or tissue. Moreover, dosages may be administered by one or more separate administrations, or by continuous infusion. For repeated administrations over several days or longer, depending on the condition, the treatment is sustained until a desired suppression of disease symptoms occurs. However, other dosage regimens may be useful. The progress of this therapy is easily monitored by conventional techniques and assays.

### Kits

The instant disclosure also provides kits containing agents of this disclosure for use in the methods of the present disclosure. Kits of the instant disclosure may include one or more containers comprising, e.g., agents for obtaining and/or assessing single cell transcriptional profiles and/or agent(s) for administration to a subject identified as responsive to such agent(s).

Where a therapeutic agent is included in the kit, the instructions generally include information as to dosage, dosing schedule, and route of administration for the intended treatment. The containers may be unit doses, bulk packages (e.g., multi-dose packages) or sub-unit doses. Instructions supplied in the kits of the instant disclosure are typically written instructions on a label or package insert (e.g., a paper sheet included in the kit), but machine-readable instructions (e.g., instructions carried on a magnetic or optical storage disk) are also acceptable.

The kits of this disclosure are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (e.g., the container may be an intravenous solution bag or a vial having a stopper pierceable by a hypodermic injection needle). The container may further comprise a second pharmaceutically active agent.

Kits may optionally provide additional components such as buffers and interpretive information. Normally, the kit comprises a container and a label or package insert(s) on or associated with the container.

The practice of the present disclosure employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000).

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Reference will now be made in detail to exemplary embodiments of the disclosure. While the disclosure will be described in conjunction with the exemplary embodiments, it will be understood that it is not intended to limit the disclosure to those embodiments. To the contrary, it is intended to cover alternatives, modifications, and equivalents as may be included within the spirit and scope of the disclosure as defined by the appended claims. Standard techniques well known in the art or the techniques specifically described below were utilized.

### EXAMPLES

### Example 1: Materials and Methods

### Cell Line Pooling

Cell line pools were made in sets of 25 cell lines. These 25 cell line pools were chosen based on doubling time and were grown in RPMI in the absence of phenol red and with 10% FBS. Cell lines were then washed with 10 mLs of PBS, and trypsinized with 1 mL trypsin which was then removed. Ten mLs of RPMI media were added to the cells post trypsinization and resuspended. Cells were then counted by a Nexcelom cellometer using 10 uL of cell suspension and 10 ul of Trypan blue. Equal numbers of cells per cell line were mixed together and spun down at 1,250 RPM for 5 minutes. Media was aspirated and the cells were resuspended in Sigma Cell Freezing media and frozen in 1 mL aliquots. This process was repeated for all of the 25 cell line pools. For MIX-Seq experiments involving larger pools, multiple 25 cell line pools were thawed in RPMI with 10% FBS, spun down and resuspended in 5 mLs of RPMI media. Cells were then counted and equal numbers were combined together on the day of plating to form larger pools of up to ˜100 cell lines.

### Cell Culture

For drug treatment experiments, cell line pools were cultured in RPMI containing 10% fetal bovine serum, in the absence of phenol red and penicillin/streptomycin. Cell line pools were validated as Mycoplasma free prior to initiating the experiment. Cell line pools were plated at 200,000 cells per well in 6 well plates containing 2 mL of RPMI culture media described above. Cell seeding density did not vary depending on pool size (25, 50 or 100 cell line pools). Cell pools were plated ˜16-20 hours prior to drug treatment. Cells were treated with the described drugs or vehicle (DMSO) with a 0.2% final media DMSO concentration.

For GPX4 knockout, cell line pools were plated at 200,000 cells per well in 12 well plates containing 1 mL of RPMI culture media. 24 hours later, the cells were infected with lentivirus expressing Cas9 and sgRNA at a multiplicity of infection of 20 in the presence of 4 ug/mL of polybrene. At 48 hours after the infection, the culture medium was replaced with medium containing 1 ug/mL puromycin. Cells were harvested at 72 or 96 hours after the infection.

### Cell Harvesting

Generally cells were harvested after drug treatment using standard cell culture methods. Briefly, after drug treatment cells that were in suspension (presumably containing dead cells from drug treatment) were collected and reserved for addition to the adherent cell fraction. Adherent cells were washed once with 1×PBS, trypsinized in 1 mL trypsin, incubated for 3-7 minutes at 37 C, and then trypsin inactivated with 1 mL growth media. For Cell Hashing, cells were treated with TrypLE Express (ThermoFisher) instead of trypsin to reduce the amount of cell surface proteins digested that may affect the binding of Cell Hashing antibodies.

For the trametinib time course experiment, cells were treated with trametinib with a staggered dosing schedule so all timepoints could be collected simultaneously. Cells were plated 19 hours prior to the first drug treatment, corresponding to the 48 hour time point. Cells were harvested for 10× capture ˜67 hours after initial seeding. Final concentrations for drug treatments are listed in Table 1 below.

TABLE 1

| | Concentration | Timepoint | |
|---|---|---|---|
| Drug | (uM) | Assayed (Hrs.) | Source |
| Idasanutlin | 2.5 | 6, 24 | Med Chem Express |
| Bortezomib | 2.5 | 6, 24 | EMD Millipore |
| Navitoclax | 5 | 24 | Selleck Chemicals |
| BRD-3379 | 10 | 6, 24 | Custom synthesis |
| Dabrafenib | 0.1 | 24 | Selleck Chemicals |
| Everolimus | 10 | 24 | Sigma-Aldrich |
| Afatinib | 0.5 | 24 | Selleck Chemicals |
| Prexasertib | 1 | 24 | Selleck Chemicals |
| Taselisib | 1 | 24 | Selleck Chemicals |
| AZD5991 | 2 | 24 | ChemieTek |
| JQ1 | 2.5 | 24 | Med Chem Express |
| Gemcitabine | 0.1 | 24 | Selleck Chemicals |
| Trametinib | 0.1 | 3, 6, 9, 12, 24, 48 | Selleck Chemicals |

Preparation of Cell Suspensions and scRNA-Seq

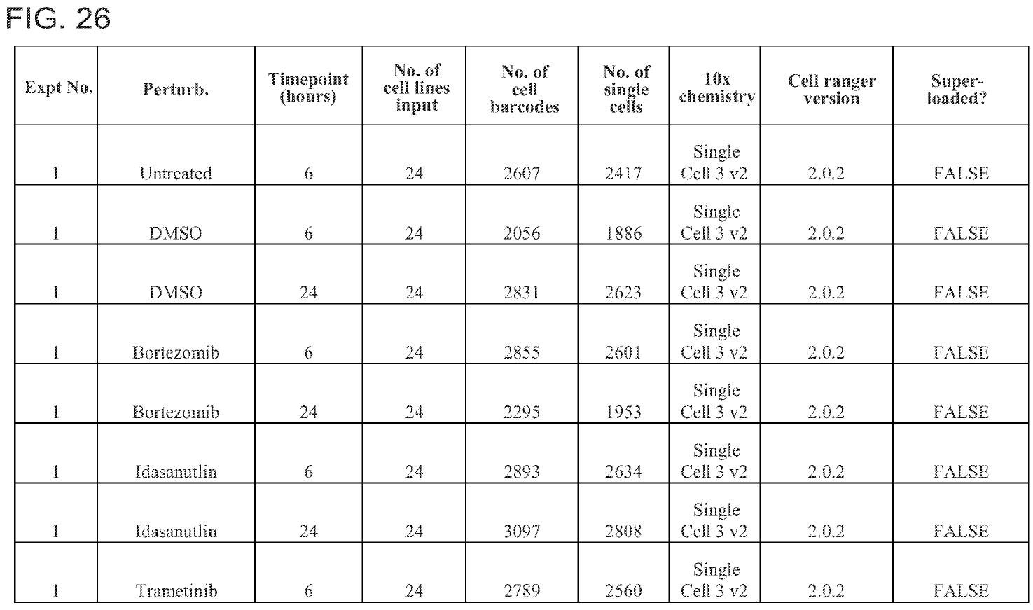

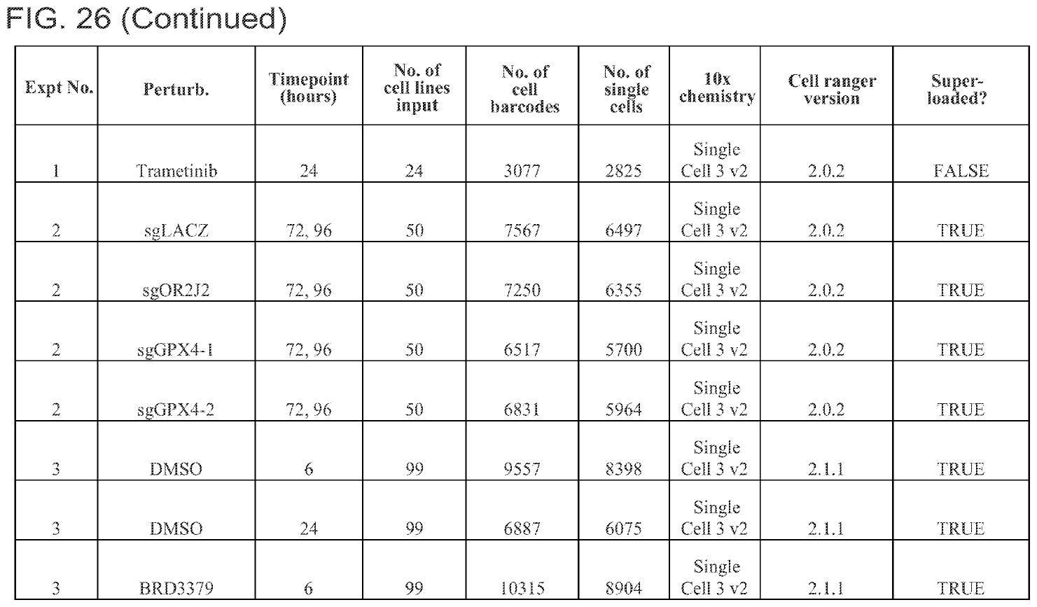

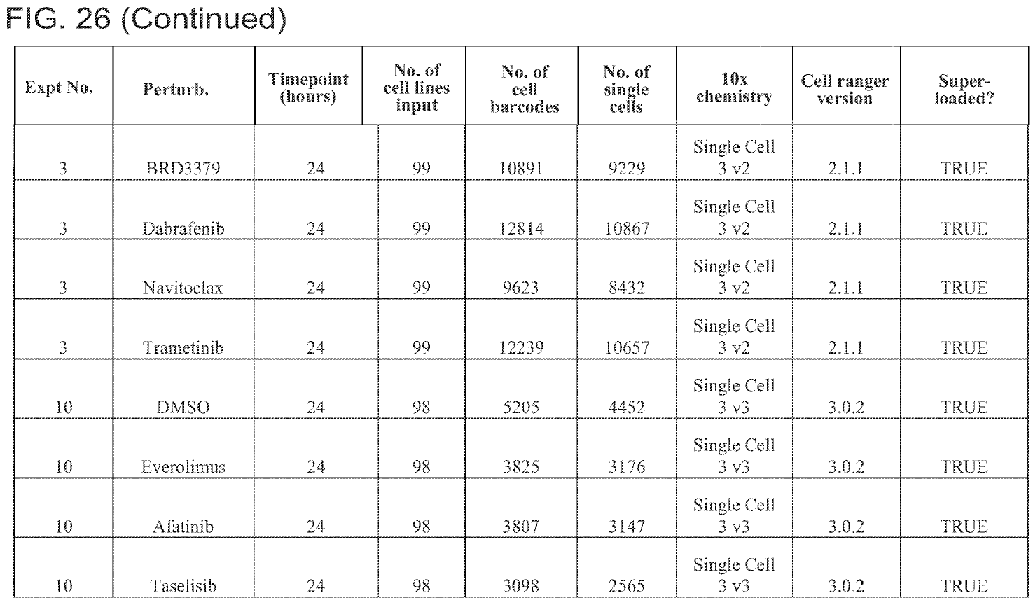

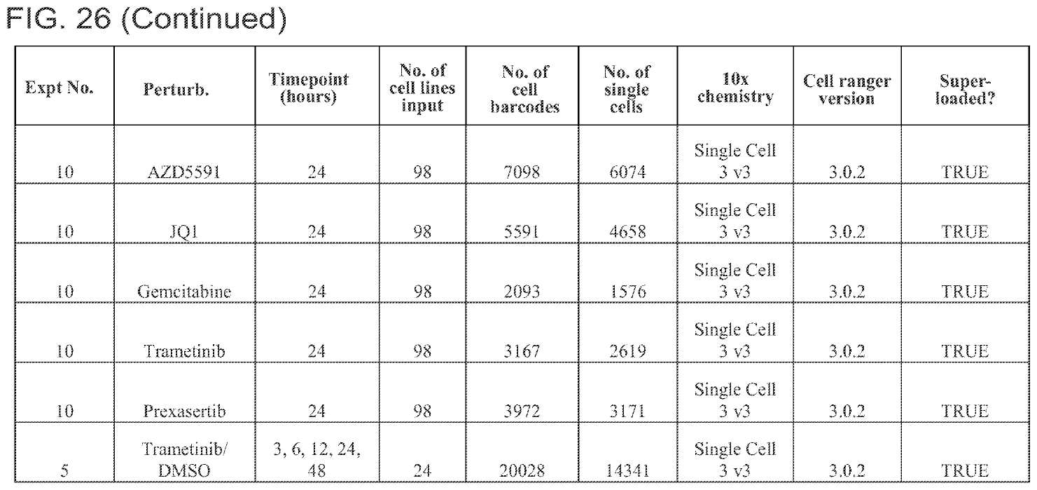

After trypsinization, adherent and suspension cells were combined for each treatment, pelleted, and resuspended in Cell Capture Buffer (lx PBS with 0.04% BSA). Cells were counted (including trypan blue non-viable cells) and resuspended at a concentration of 1,000 cells per microliter for standard loading on the Chromium Controller (10× Genomics), or at 1,500 cells per microliter for “super loaded” samples. Up to 40,000 cells were loaded per 10× channel for “super loaded” samples, with expected recovery of up to 20,000 cells per channel. Cell suspensions were captured on a 10× Chromium controller using Single Cell 3′ reagent chemistries (either version 2 or and version 3 reagents) FIG. 26.

### Cell Hashing Cell Labelling

Cell Hashing (Stoeckius et al., 2018) was performed using the cell harvest method described above with the following changes. All steps were performed on ice. Harvested cells were resuspended in Cell Hashing Staining Buffer (lx PBS with 2% BSA and 0.02% Tween) prior to cell counting. Samples were counted in duplicate with two technical replicates by Countess (Life Technologies) to estimate total cell number. Up to 1,000,000 cells (range 3e5-1e6 cells) were resuspended in 100 microliters of Cell Hashing Staining Buffer. Cells were blocked with 10 microliters of Human TruStain FcX blocking solution (BioLegend) for 10 minutes at 4° C. 100 microliter cell suspensions in Cell Hashing Staining Buffer were then incubated with 2 μL of the appropriate BioLegend TotalSeq™-A Hashing antibody A Hashing antibody (a 1:50 dilution, using a total of 1 μg of antibody per cell suspension). TotalSeq™-A anti-human Hashtag antibodies #4-10 and 12-15 (product codes: 394607, 394609, 394611, 394613, 394615, 394617, 394619, 394623, 394625, 394627, 394629) were used. Cells were washed three times with 0.5 mL of Cell Hashing Staining Buffer and filtered through low volume 40 μm cell strainers (Flowmi). All cell suspensions were recounted to achieve a uniform concentration of 1,500 cells per microliter before pooling for 10× capture. A detailed general protocol is available (www.protocols.io/private/4AC0F6594480B498D8B60EAF6F518E66).

### Cell Hashing Library Preparation

Separation of hashtag oligo (HTO)-derived cDNAs (<180 bp) and mRNA-derived cDNAs (>300 bp) was done after whole transcriptome amplification by performing 0.6×SPRI bead purification (Agencourt) on cDNA reactions as described in 10× Genomics protocol. Briefly, the supernatant from 0.6×SPRI purification contains the HTO fraction, which was subsequently purified using two 2×SPRI purifications per manufacturer protocol (Agencourt). HTO's were eluted by resuspending SPRI beads in 15 μl TE.

Purified HTO sequencing libraries were then amplified by PCR. PCR reactions were as follows:

| Reagent | Volume |
|---|---|
| Purified HTO fraction after 2X SPRI | ~1 μL (5 ng) |
| 2x NEB Next Master Mix | 25 μL |
| Illumina TruSeq DNA D7xx_s primer | 1 μL |
| (containing i7 index) 10 μM | |
| SI PCR oligo 10 μM | 1 μL |
| H₂O | To 50 μL final volume |

Typically 3 identical “dial out” PCR reactions were performed per HTO library. The number of PCR cycles were varied to avoid under or overamplifying the HTO libraries. PCR cycling conditions were are follows:

| Step | Temperature | Time |
|---|---|---|
| 1 | 98° C. | 10 sec |
| 2* | 98° C. | 2 sec |
| 3* | 72° C. | 15 sec |
| 4 | 72° C. | 1 min |

*Steps 2&3 were repeated for 15, 18, or 22 cycles

PCR reactions were purified using another 2×SPRI clean up and eluted in 15 uL of 1×TE. HTO libraries were then analyzed for amplification quality. Libraries were quantified by Qubit High sensitivity DNA assay (ThermoFisher) and loaded onto a BioAnalyzer high sensitivity DNA chip (Agilent) to determine if an intended HTO product size of ˜180 bp was achieved.

### Sequencing

Samples were sequenced using HiSeq X (Illumina) and NovaSeq 6000 (Illumina) platforms. The read structure (for 10×3′ v3 chemistry) was as follows:

| Platform | Read | Cycles |
|---|---|---|
| HiSeq | Read 1 | 28 (26)* |
| | Read 2 | 96 |
| | Index 1 | 8 |
| NovaSeq | Read 1 | 28 (26)* |
| | Read 2 | 80 |
| | Index 1 | 8 |

*For 10x 3′ v2 chemistry

The hashing library for the trametinib time-course experiment was sequenced twice with spike-ins of 2.5-10%.

### Data Processing

Sequencing data were processed using 10× Cell Ranger software, run using the Cumulus cloud-based analysis framework (Li et al. 2019). Initial experiments were done with 10× Single Cell 3′ v2 chemistry, and were processed using version 2 of the Cell Ranger software. In later experiments v3 chemistry and the corresponding version 3 of Cell Ranger were used. Reads were aligned to the hg19 reference genome.

### SNP Identification

To define a SNP panel for cell line classification, SNPs were identified that occurred frequently across a large panel of 1,160 cell lines. Specifically, MuTect 1 (version 1.1.6) was employed to call SNVs from bulk RNA-seq data, as well as single-cell RNA-seq data, for 200 cell lines, using a downsample to coverage rate of 1,000, and a fraction contamination rate of 0.02. All other parameters were set to defaults. The subset of SNPs that were observed in both the bulk and single cell data were selected, then all SNPs were ordered by frequency of occurrence in the bulk RNA-seq data. From this the 100,000 most frequently observed SNPs were selected.

For the bulk RNA-seq data, Freebayes (Garrison et al. 2012) was used to estimate allelic fractions across the reference SNP panel, using the settings “pooled-continuous” and “report-monomorphic”. A pseudocount of 1 was added to the reference and alternate allele read counts. For the single cell data, the method scAlleleCount (www.github.com/barkasn/scAlleleCount) was used to extract reference and alternate allele counts at all SNP sites.

### SNP-Based Cell Line Classification

A generalized linear modeling approach was used to estimate the likelihood of the observed SNP reads for an individual cell having come from each parental cell line. Specifically, a logistic regression model was employed, wherein the probability of a read at SNP site i being an alternate allele was given by:

πᵢ=σ(β₀+βⱼXᵢⱼ), (1)

wherein σ is the logistic function, Xᵢⱼ is the (predefined) fraction of reads at SNP site i from the alternate allele in cell line j (estimated from bulk RNA-sequencing data), and β are parameters estimated for each single cell and reference cell line by maximizing the likelihood:

$${\prod }_{i} ℒ ( β \vert {y}_{i} , {n}_{i} ) , \qquad ( 2 )$$

wherein [symbol] is the binomial likelihood, yᵢ is the number of alternate reads, and nᵢ is the total reads observed at site i. Models were fit using the glm function in R, and the cell line whose SNP allelic fraction profile Xⱼ produced the highest likelihood for the observed single-cell SNP reads was selected. Goodness-of-fit was quantified by the deviance ratio: 1−deviance/(null deviance). A measure of the classification confidence was also computed, given by the margin between the best-fitting and second-best fitting model deviance ratios, and normalized by the standard deviation of deviance ratio values across reference cell lines (excluding the best matching cell line j*).

Estimates of the SNP classification error rate were given by

$$\frac{{n}_{out}}{{n}_{tot}} c ,$$

where nₒᵤₜ is the number of cells erroneously classified as out-of-pool cell lines, nₜₒₜ is the total number of cells recovered in the experiment (excluding doublets and low-quality cells), and c is a correction factor to account for the probability of cells being classified incorrectly among the in-pool cell lines. Assuming errors were made with equal probability among all reference cell lines, this is given by

$$c = 1 - \frac{{N}_{pool} - 1}{{N}_{ref}} . \qquad ( 3 )$$

### Modeling Doublets

Doublet detection was performed using a similar generalized linear modeling approach, where alternate allele probabilities were modeled as a mixture of the allelic fraction profiles from two reference cell lines Xⱼ and Xₖ:

πᵢ=σ(β₀+βⱼXᵢⱼ+βₖXᵢₖ), (4)

wherein the ratio βⱼ/βₖ represented the proportion of mRNA reads from cell line j vs cell line k. To efficiently estimate the most likely pairwise mixture of reference cell lines, a Lasso-regularized generalized linear model was used (implemented in the R package glmnet (Friedman et al. 2010)), with the allelic fraction profiles for all in-pool reference cell lines Xⱼ considered as covariates. Coefficient estimates were constrained to be non-negative, and the model was limited to use of a maximum of 2 non-zero coefficients (i.e. 2 reference SNP profiles). After using the Lasso model to estimate the most-likely ‘doublet’ pair of cell lines, the GLM was refit without regularization to estimate the goodness-of-fit of the doublet model (deviance), as well as the optimal mixing ratio. To measure the evidence in favor of a cell being a doublet, the difference of deviance ratios of the best-fit doublet and singlet models was employed (equivalent to the log likelihood ratio of the doublet and singlet models, normalized by the log likelihood ratio between the saturated and null models).

### Classifying Low-Quality Cells and Doublets

To identify low-quality cells and classify doublets, cells were initially removed which exhibited a high or low proportion of UMIs from mitochondrial genes (>0.25 or <0.01), or with reads at fewer than 50 of the reference SNP sites. In many experiments, groups of cells were observed that exhibited distinct gene expression profiles and SNP profiles that did not match to any reference cell line (or pairwise combination of cell lines) in particular, but rather resembled more a mixture of SNPs from all the in-pool cell lines, which indicated that these were empty droplets containing ambient mRNA in the pool (Macosko et al. 2015; Stoeckius et al.). To identify these putative empty droplets, the single-cell gene expression profiles were first clustered using Seurat's default graph-based clustering with 10 nearest neighbors and a resolution parameter of 1-4 (depending on the pool size). Gene expression clusters were then identified which consistently exhibited poor-fitting SNP models (i.e. that did not resemble singlets or doublets based on their SNPs). For this, the overall SNP model goodness-of-fit for each cell was assessed by the deviance ratio of the doublet model, which was strictly greater than or equal to that of the restricted singlet model. The median SNP-model deviance ratio was computed for each gene-expression cluster; and clusters with a median deviance ratio of less than 0.3 were considered to be low quality and were removed from the data before further analysis.

Doublets were then separated from singlets using a 2-component Gaussian mixture model (GMM) fit with two features: the singlet model deviance ratio, and the doublet model fit (difference in deviance ratios relative to the singlet model). GMMs were fit using the R package MClust (Scrucca et al., 2016), with the default conjugate prior on the covariance matrices, and no shrinkage on the component means. Cells with a probability >0.5 of being doublets were then taken to be doublets.

Finally, to ensure cells labeled singlets were confidently identified, the difference in goodness-of-fit between the best-fitting and second best-fitting reference cell lines was required to be at least 2 z-score. Cells that were excluded based on any of the above criteria (other than doublets) were labeled low-quality′ (FIGS. 6A-6C).

### Visualizing Single-Cell Expression Profiles

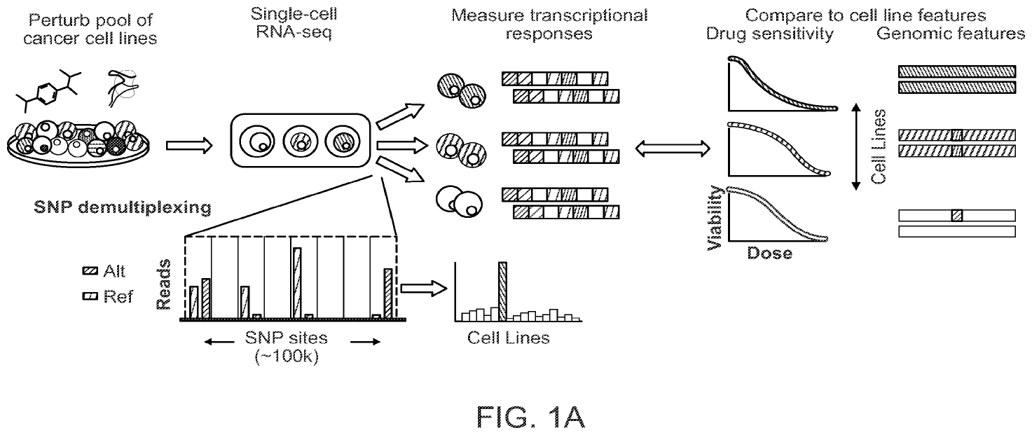

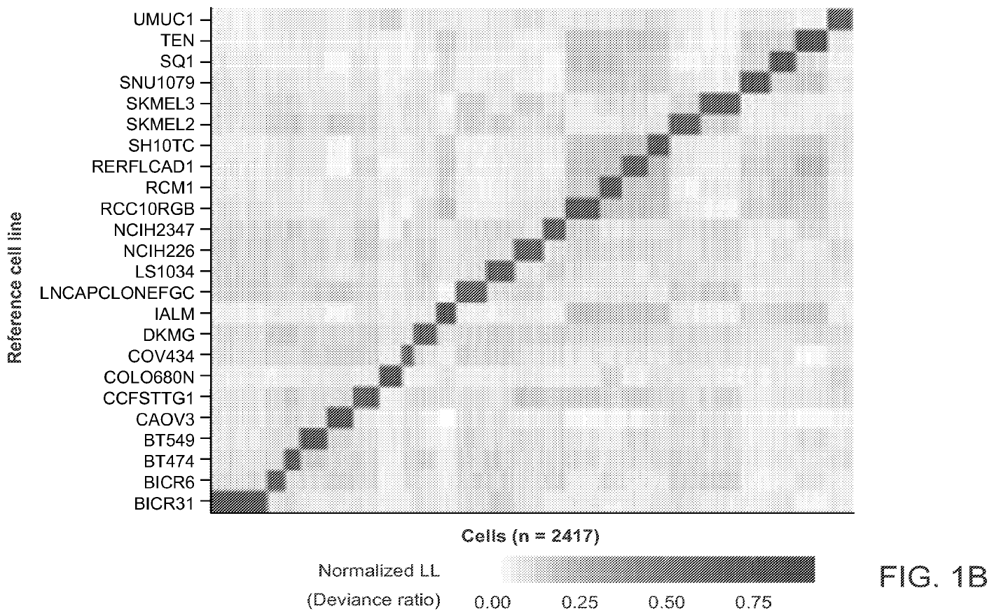

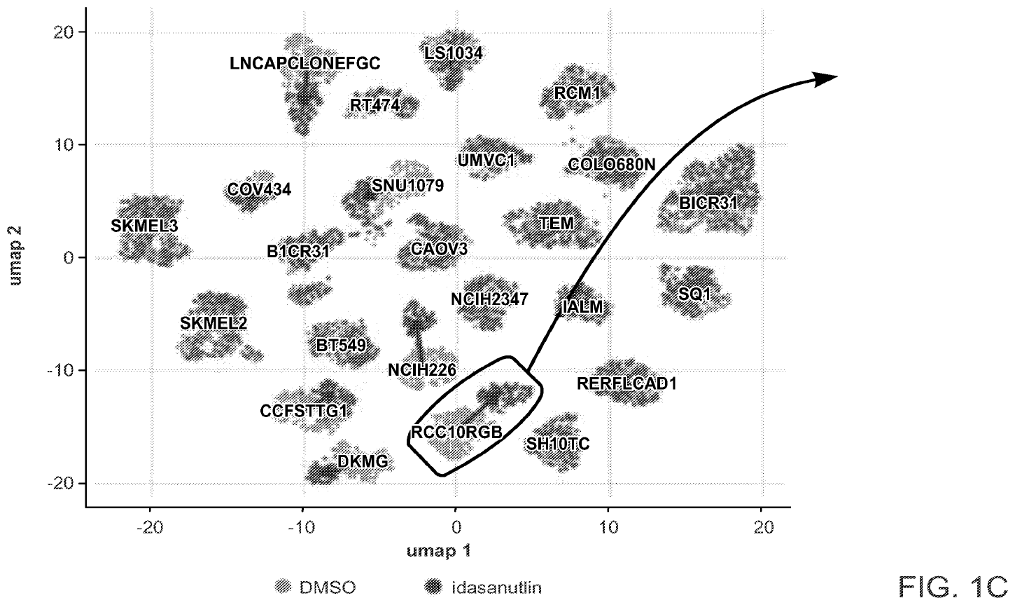

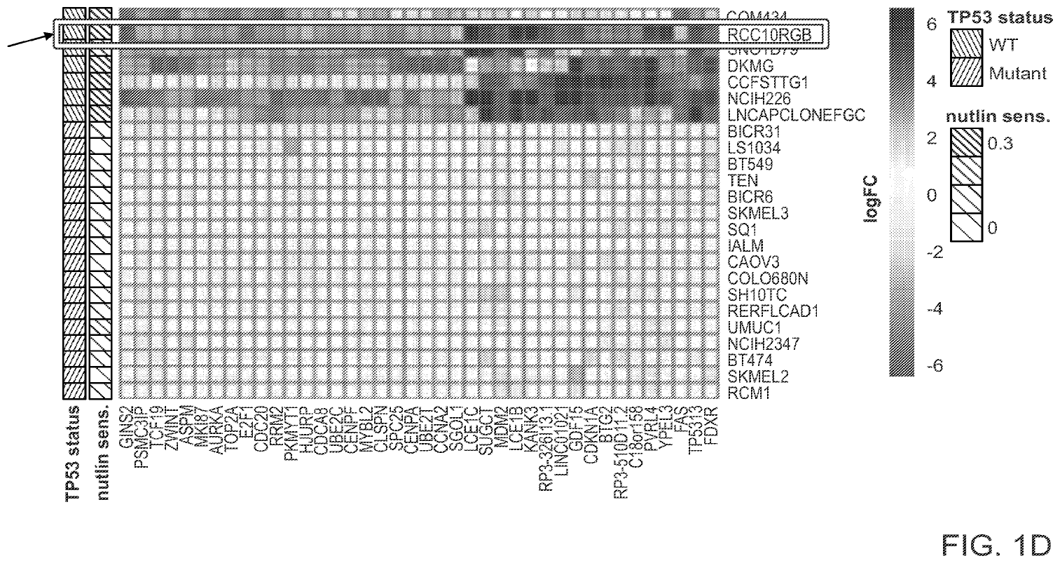

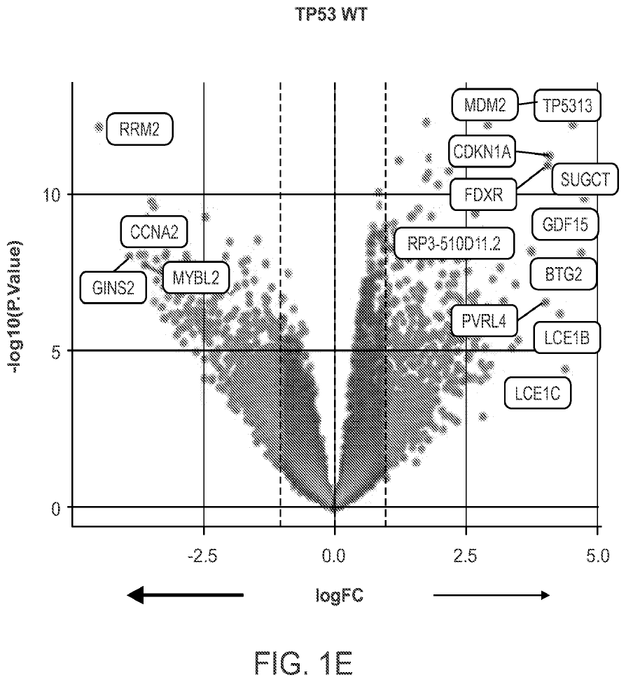

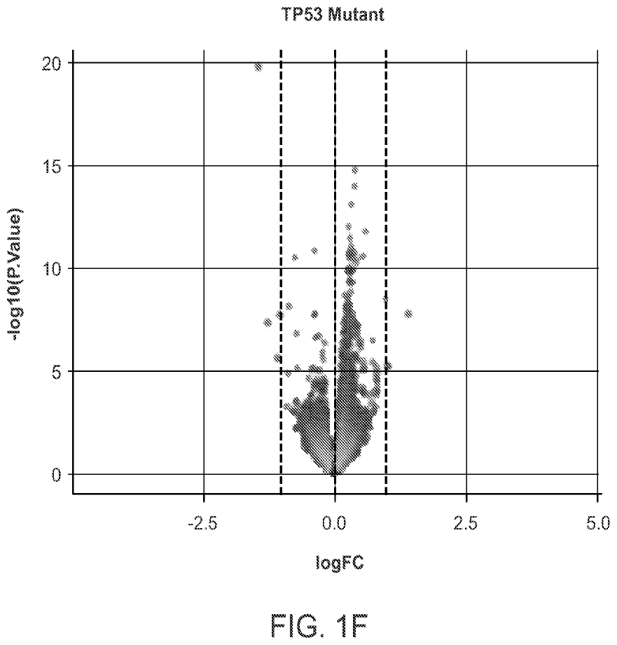

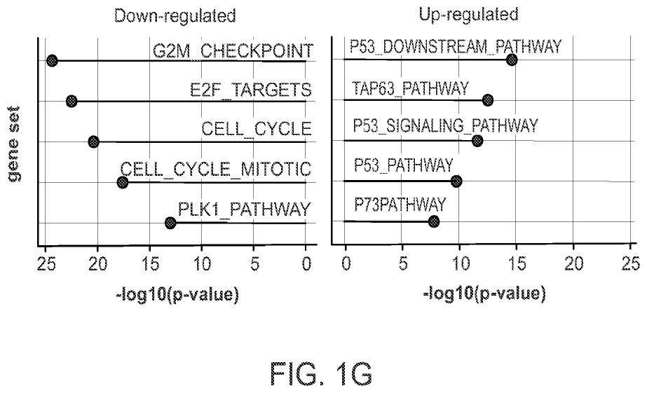

2D representations of single-cell expression profiles (e.g., FIG. 1B) were generated using Seurat v3 (Butler et al. 2018). Single-cell counts data were first normalized and log-transformed using the NormalizeData function, with a scale factor of 1e5. Data were then normalized across cells using the ScaleData function. The top 5,000 most variable genes (based on the ‘vst’ selection method) were selected using the FindVariableGenes function, and principal components were computed using the RunPCA function, retaining the top 2N PCs, where N is the number of cell lines in the pool. t-SNE embeddings were computed based on the PCs, using the RunTSNE function with a perplexity parameter of 25. UMAP embeddings were computed using the RunUMAP Seurat function with 15 nearest neighbors, and a ‘min.dist’ parameter of 0.6-1.0 (with otherwise default parameters).

### Differential Expression Analysis

To estimate the average transcriptional response of each cell line to a perturbation, the data were first ‘sum-collapsed’—summing read counts across cells for each cell line and treatment condition—to produce a bulk RNA-seq style read counts profile for each sample (Crowell et al. 2019; Lun & Marioni 2017). Normalization factors were then computed per sample (per cell line and condition) using the “TMMwzp” method from the edgeR R package (Robinson et al. 2010), and the profiles were transformed to log counts-per-million (using a ‘pseudo count’ of 1) using the edgeR function ‘cpm’ before computing the log-fold-change difference in relative gene abundances between treatment and control conditions.

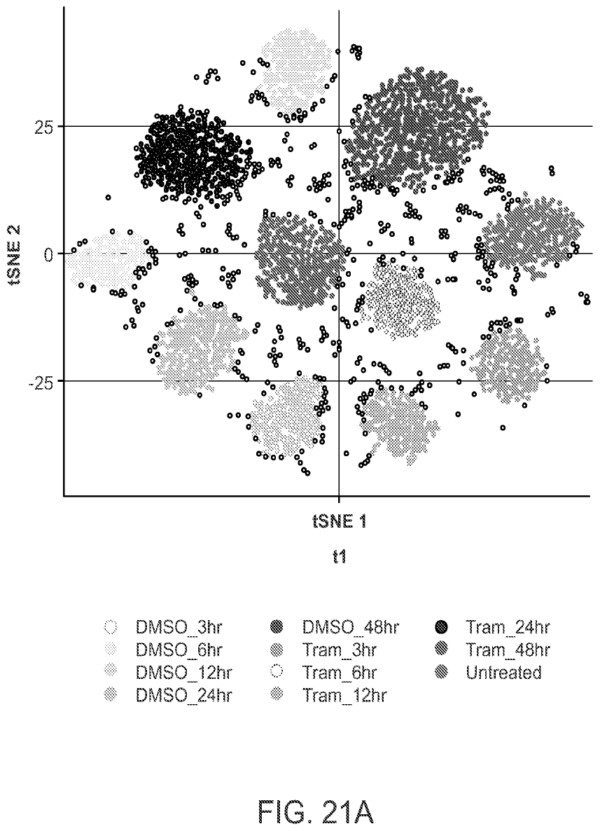

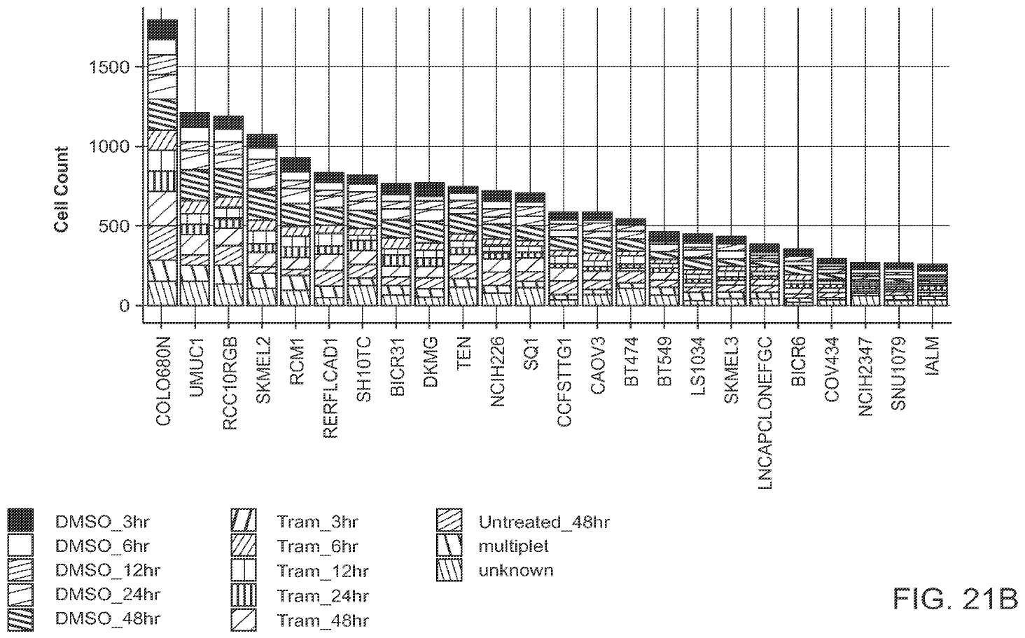

Differential expression analyses across cell lines was performed using the “limma-trend” pipeline (Law et al. 2014; Ritchie et al. 2015), applied to these sum-collapsed and normalized profiles. For this analysis, data were included from cell both 6 and 24 hours post-treatment with vehicle control (DMSO) in the control group, as no consistent time-related effect of DMSO treatment was observed in the data (FIGS. 21A and 21B). Global differences between the two control conditions were incorporated into the model to help mitigate batch effects (Lun et al. 2017).

To identify the average drug response across cell lines, models were employed of the form:

Y_{gjk}β_{g}Iₖ+c_{gk}+b_{jg}, (5)

where Y_{gjk} is the log CPM expression level of gene gin cell line j and condition k, is modeled as a sum of several terms. The first term captures the average treatment effect, where β_{y} is the average LFC of gene g, in response to treatment and Iₖ is an indicator variable representing whether condition k is treatment or control. c_{gk} captures differences in average expression across control conditions, and b_{jg} captures the baseline expression of gene g in cell line j.

To estimate the viability-related and viability-independent response components, a similar modeling approach was employed, including the measured drug sensitivity of each cell line as a covariate interacting with treatment as follows:

Y_{gjk}β_{0g}Iₖ+sⱼIₖβ_{1g}+c_{gk}+b_{jg}, (6)

where sⱼ is the measured sensitivity of cell line j to the treatment (one minus the area under the dose-response curve), β_{0g} is the viability-independent response of gene g to treatment, and β_{1g} is the viability-related response of gene g to treatment.

Only genes with at least 5 reads detected (summed across cells) in at least 5% of the samples were included in the analysis. p-values were derived from empirical-Bayes moderated t-statistics, and FDR-adjusted p-values were obtained using the Benjamini-Hochberg method (Bejamini et al. 1995).

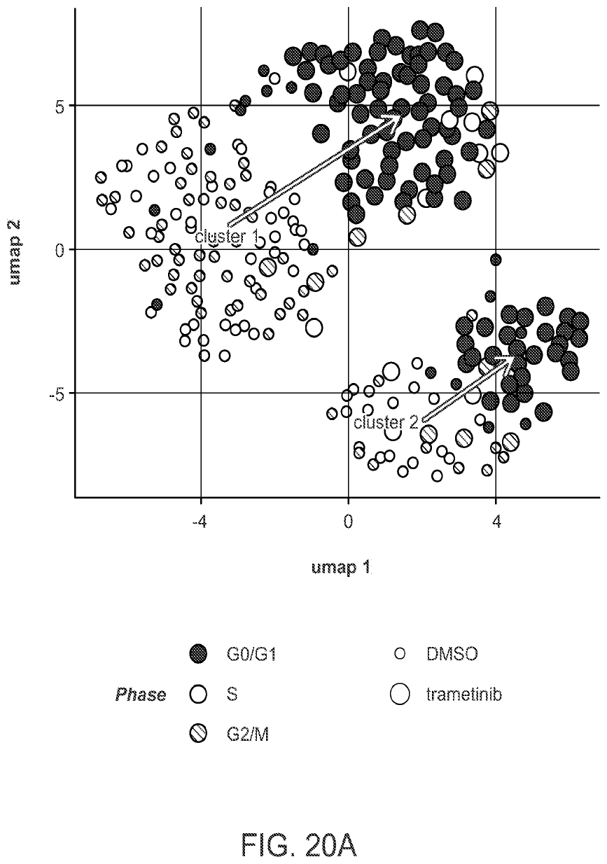

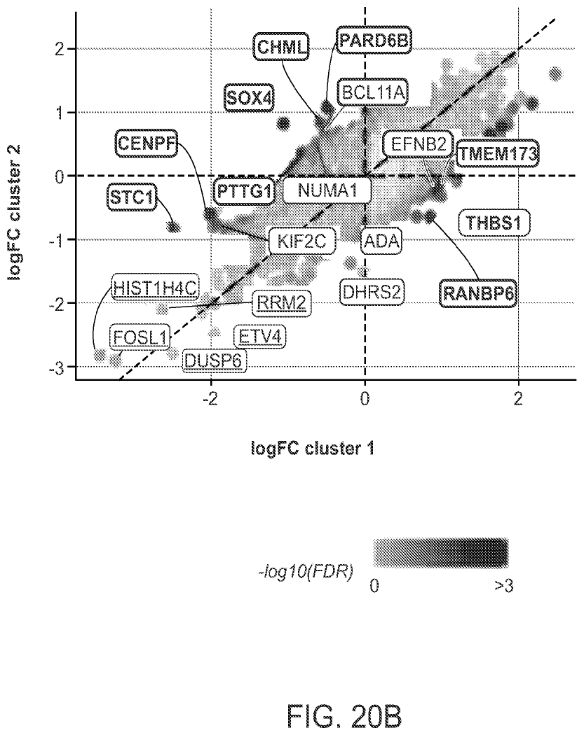

When comparing the transcriptional responses of two cell lines to a drug or cell populations to a drug (e.g., FIGS. 20A, 20B, 24A and 24B) the above method could not be applied as each group had a single sample. Hence, uncollapsed single-cell expression profiles were compared. Specifically, the edgeR quasi-likelihood approach was used (Lun et al. 2016), following the pipeline in Soneson et al. 2018, with the cell detection rate (the fraction of genes with non-zero reads detected) included as a covariate.

### Drug Sensitivity Data

Cell line drug sensitivity data were taken from the Sanger GDSC dataset (Iorio et al. 2016; Garnett et al. 2012), as well as data generated using the PRISM multiplexed drug screening platform (Corsello et al. 2020 and Yu et al. 2016). For most compounds, the area under the dose-response curve (AUC) was used to measure sensitivity. When data were available from both PRISM and GDSC datasets for a given drug, the average of each cell line's AUC values were used, after quantile normalization of the AUC measurements from each dataset.

For nutlin treatment, nutlin-3a data from GDSC was combined with nutlin-family compound idasanutlin (RG7388) PRISM data. For the tool compound BRD3379, the (PRISM) data were most reliable for the highest dose, so log viability measurements at a single dose of 10 μM were used, though results were similar when using the AUC.

### Gene Set Enrichment Analysis (GSEA)

For analysis of gene set enrichment of transcriptional response signatures, a simple approach was used, measuring the set overlap (Fisher's-exact test) between each gene set and the 50 top up- and down-regulated genes (based on the estimated log-fold-change). The collection of gene sets used was the combination of the ‘Hallmark’ and ‘Canonical’ gene set collections from MSigDB v6.2 (Liberzon et al. 2011).

### Estimating Relative Cell Line Abundance

Estimates of the effects of perturbations on relative cell line abundance were obtained by counting the number of (QC-passing) single cells from each cell line in each treatment condition, adding a ‘pseudo-count’ of 1, and normalizing counts across cell lines per condition. These relative abundance estimates were averaged across samples for each treatment condition to compute the log₂-fold-change difference between drug-treated and control relative cell line abundances.

### Cell Cycle Analysis

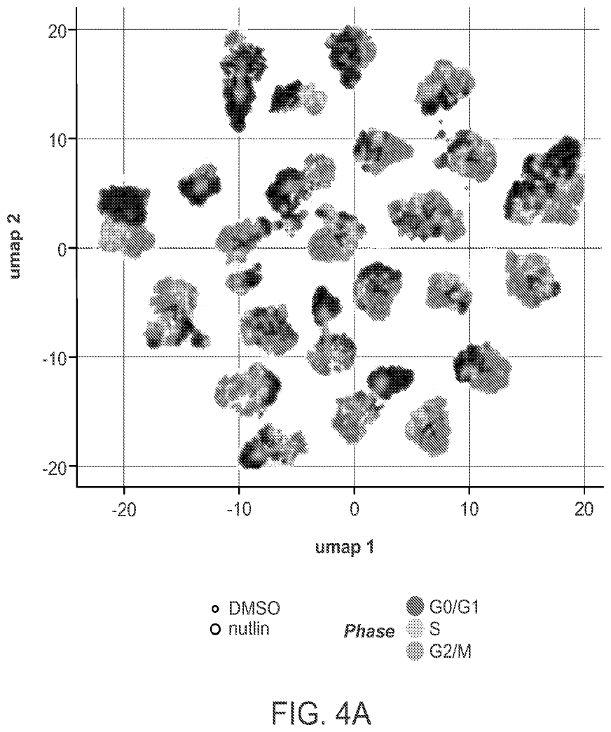

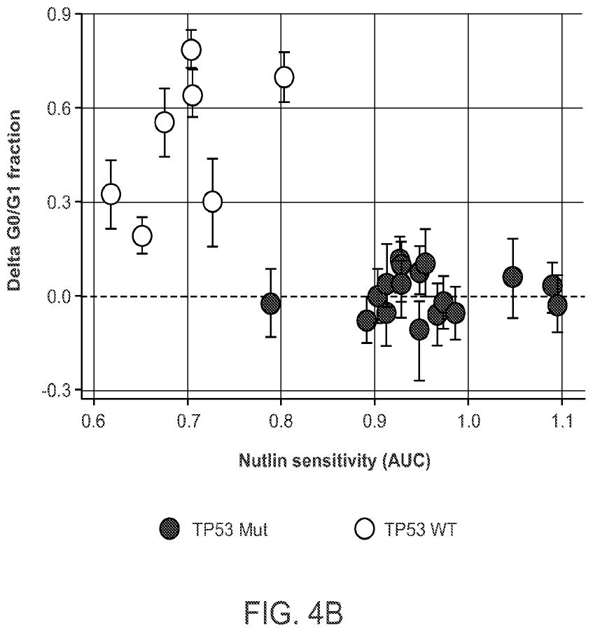

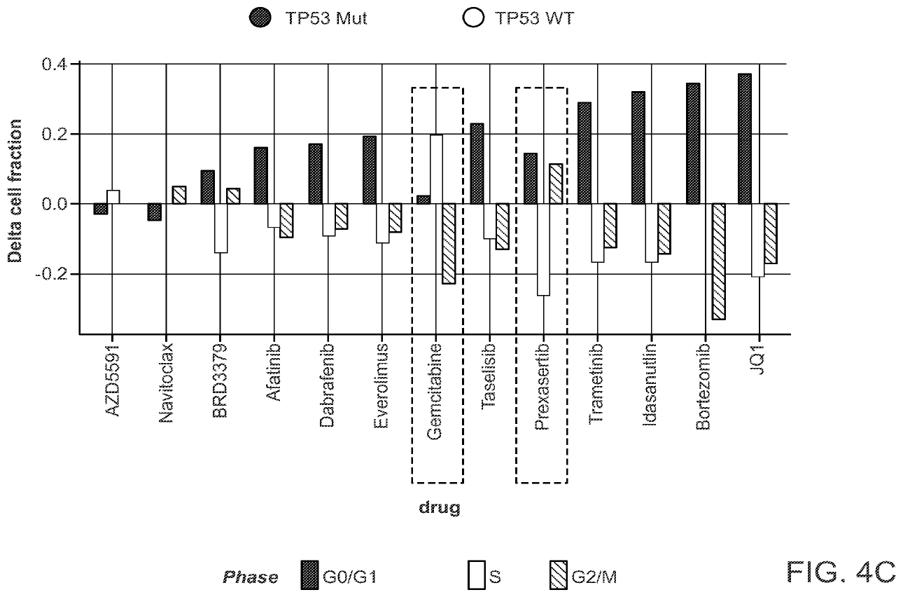

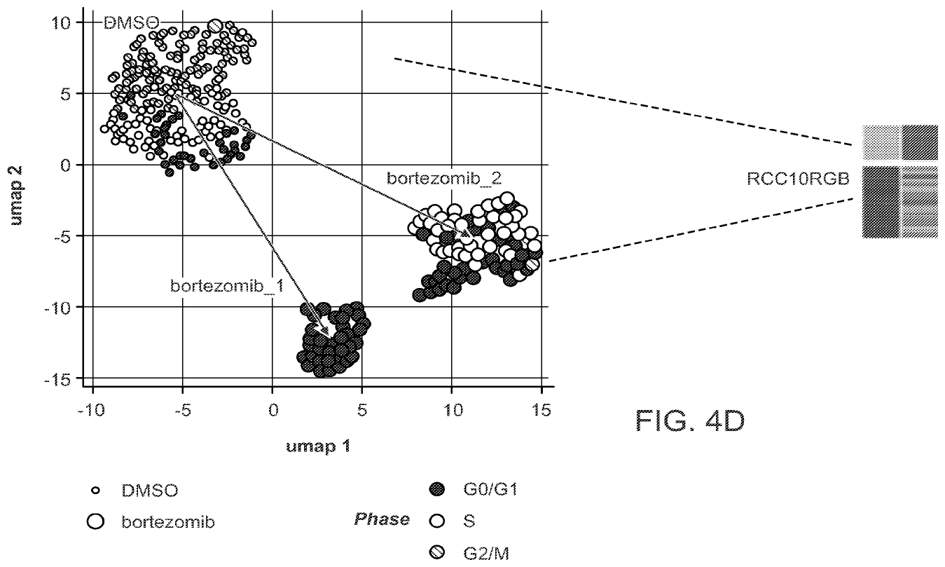

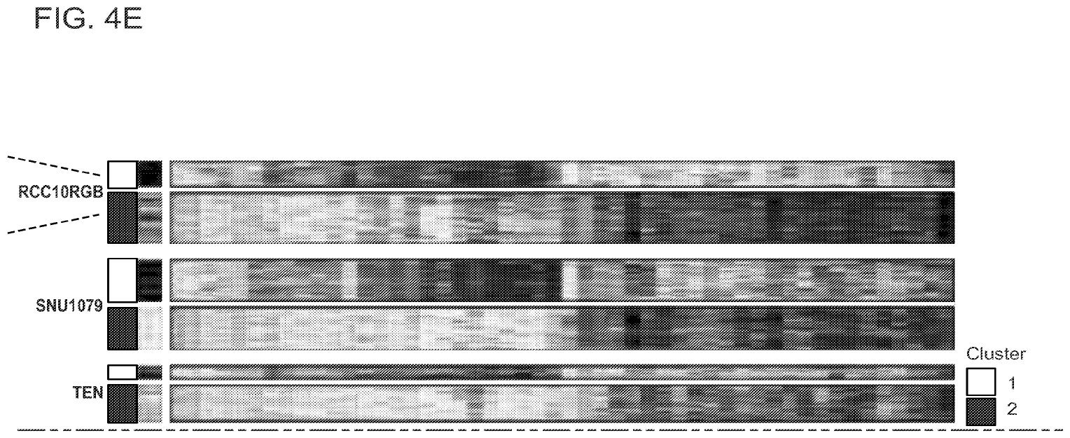

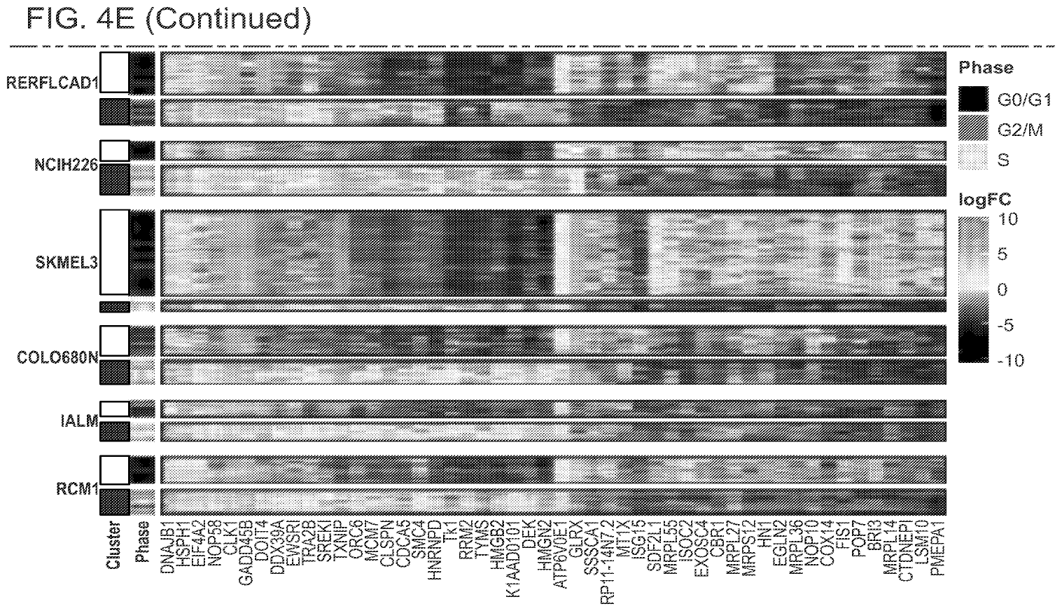

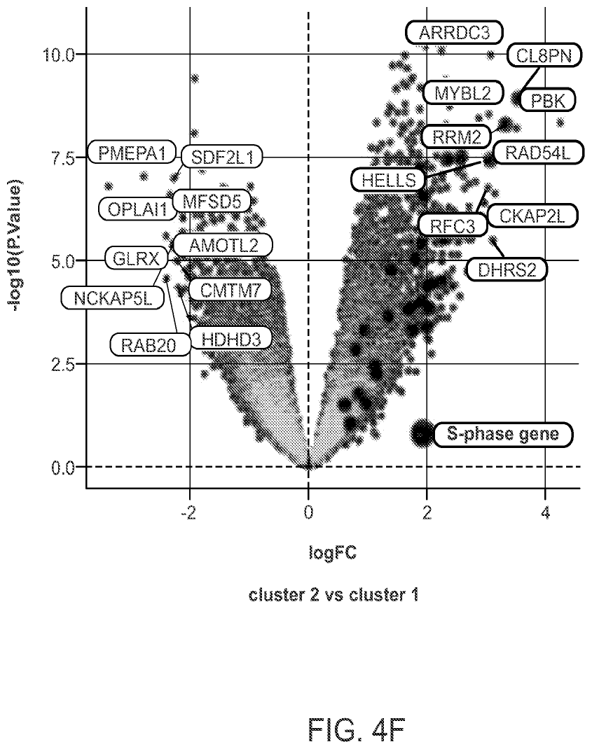

Cell cycle phase classification was performed using the Seurat function CellCycleScoring, using the S- and G2M-phase gene lists reported in Tirosh et al. 2016. The change in proportion of cells in each phase between treatment and control conditions for each cell line, along with associated confidence intervals, were estimated using the prop.test R function. For FIG. 4C, aggregate scores were computed representing how each compound altered the cell cycle composition by computing weighted averages across cell lines of the change in proportion of cells in each phase, where the weights were determined by the cell lines' measured drug sensitivity (1 minus AUC, bounded between 0 and 1).

### Principal Component Analysis

For principal component analysis (PCA) and other machine learning analyses, a slightly different procedure was used to estimate each cell line's average transcriptional response to drug treatment. Rather than sum-collapsing the read count data, the single-cell gene expression profiles were mean-collapsed by normalizing each single-cell profile to counts-per-million, averaging across cells, and then log-transforming the averaged profiles (using a larger pseudo count value of 10 to help stabilize log-fold change estimates for lowly expressed genes). PCA was then computed on the matrix of cell line log-fold change profiles, mean-centered per gene, using the 5,000 genes with the most across-cell-line variance. Cell lines were included in PCA analysis if there were at least 10 cells in both control and treatment conditions.

The use of mean-collapsed, rather than sum-collapsed profiles for machine learning analysis helped prevent any bias in the estimated log-fold change responses related to the number of cells recovered for each cell line. Both sum-collapsed and mean-collapsed log-fold change estimates produced similar results, differing primarily in whether cells with greater sequencing depth are given more weight.

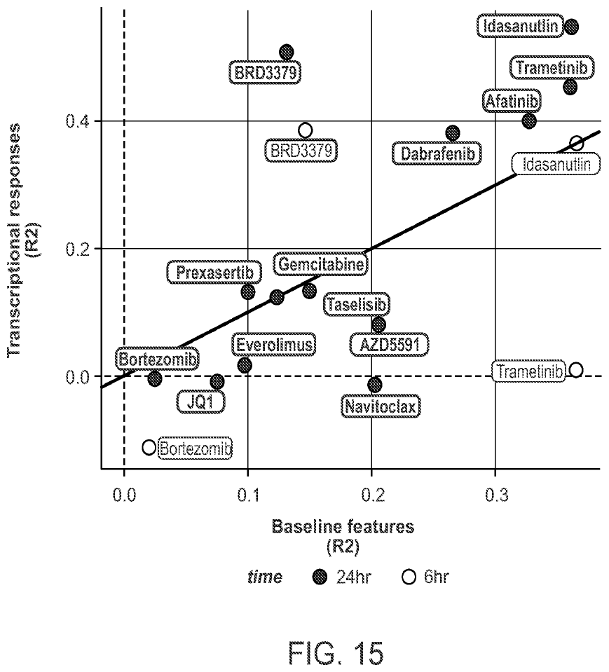

Comparisons of PCI loadings with measured drug sensitivity across cell lines (FIG. 15) were made using Pearson correlations, with p-values estimated using the ‘cor.test’ R function. FDR adjusted p-values were estimated using the Benjamini-Hochberg method (Benjamini et al. 1995).

### Transcriptional Response Embedding

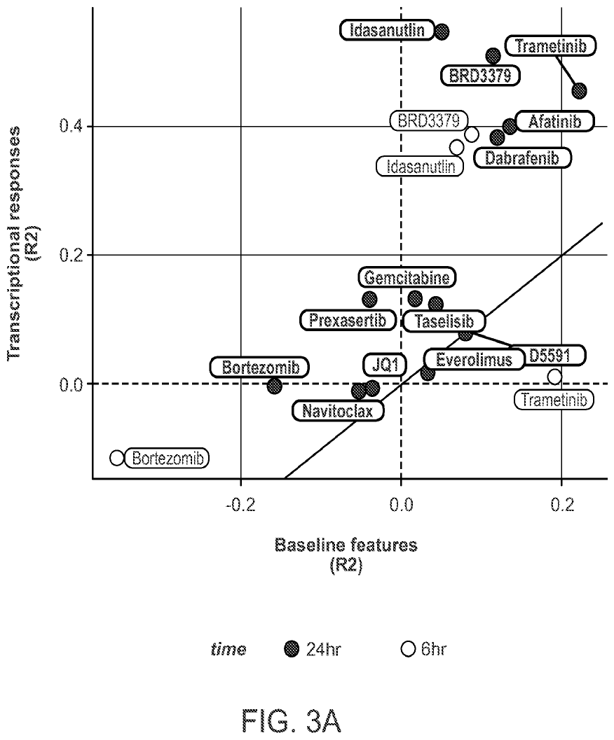

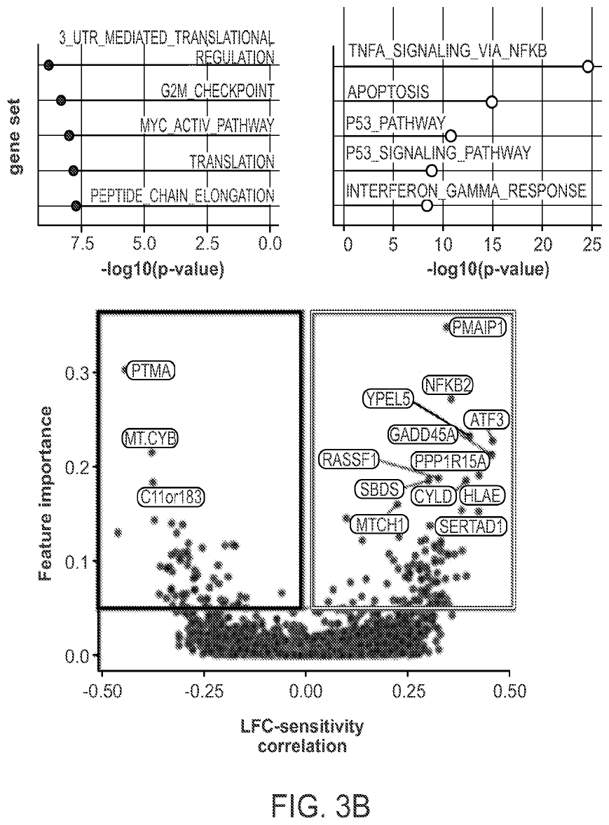

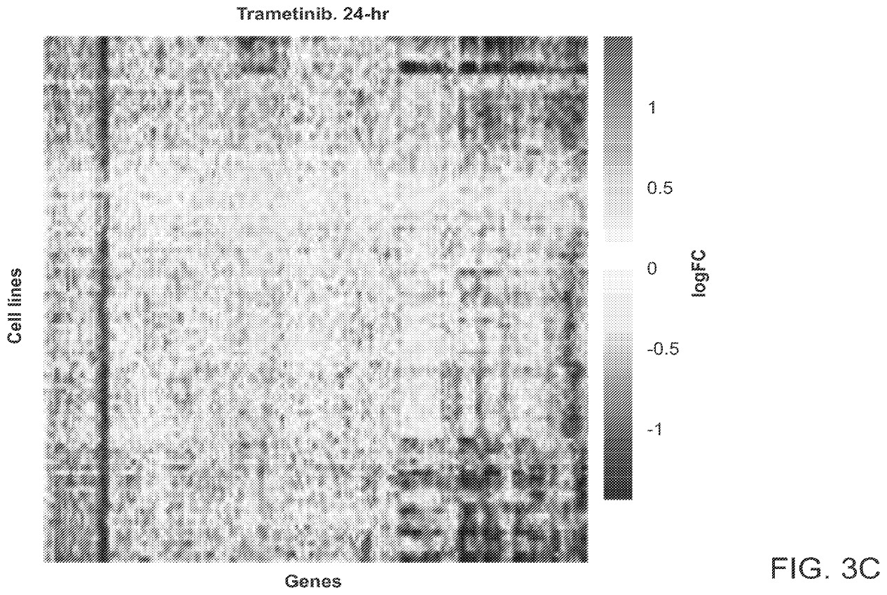

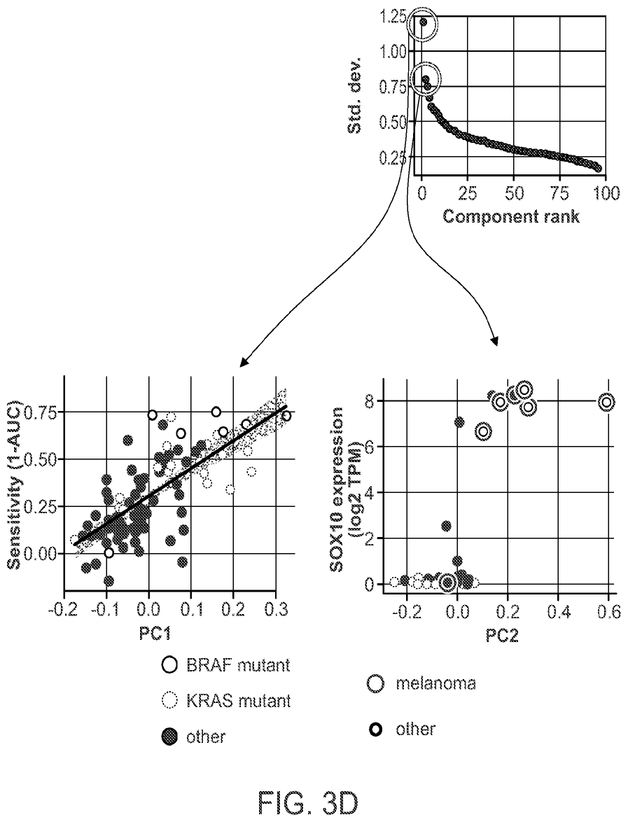

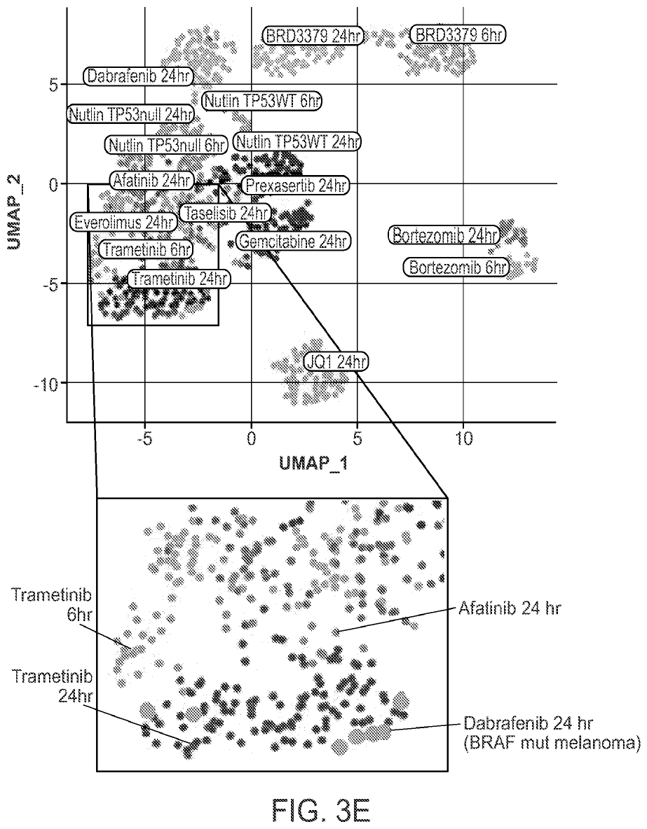

To compute the embedding of transcriptional response profiles (FIG. 3E), the UMAP method (McInnes et al. 2018) was used, as implemented in the Seurat package. Specifically, all log-fold change response profiles were compiled across cell lines and treatment conditions (computed using mean-collapsed profiles). Analysis was restricted to response profiles supported with at least 10 cells per condition and 40 cells total. 5,000 genes with highest variance were selected, and the top 30 principal components were computed. UMAP was then run using cosine distance between samples in this principle component space, with an ‘n.neighbors’ parameter of 15, and ‘min.dist’ of 0.6.

### Predictive Modeling Analysis

To assess how well a cell line's drug sensitivity could be predicted from baseline features, or measured transcriptomic responses, random forest regression models (using the R package ranger (Janitza et al. 2016) were employed, with default parameters. Prediction accuracy (R² of model predictions) was evaluated using 10-fold cross-validation. AUC values were capped at 1.5 before model training to mitigate the effects of a few outliers with large AUC values, though results were similar without capping of AUC values. To help mitigate overfitting, features were pre-filtered, selecting the top 1000 features based on the magnitude of their marginal correlation with the response variable (feature selection was performed separately for each cross-validation set, using training data only). Cell lines were only included if there were at least 5 cells per condition (treatment and control) for a given drug, to ensure that the estimated transcriptional response profiles were sufficiently robust.

To estimate the importance of transcriptional response features used by the model (FIG. 3B) the ‘impurity’ feature importance metric of the ranger package was utilized, without applying feature pre-selection.

For the baseline omics' features, baseline log TPM expression levels of each protein coding gene were used, as well as the damaging and hotspot missense mutation status of each gene (Barretina et al. 2012 and Ghandi et al. 2019). These data were taken from the DepMap 19Q3 data release, available at www.depmap.org.

### Time Course Analysis

Classification of single-cell treatment conditions, as well as doublet classification, from the hashtag read counts data were performed using DemuxEM (Gaublomme et al. 2019), with default parameters.

The same approach described above was used to estimate the viability-related and viability-independent components of the response at each time point post-trametinib treatment. As substantial transcriptional changes across time points after DMSO-treatment were not observed (FIGS. 21A and 21B), data across DMSO conditions was pooled for analysis.

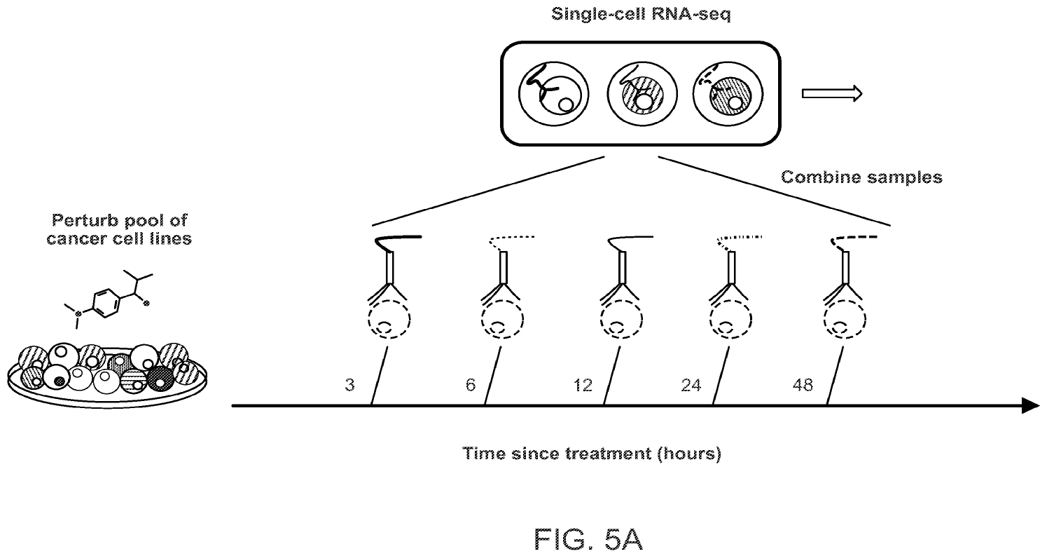

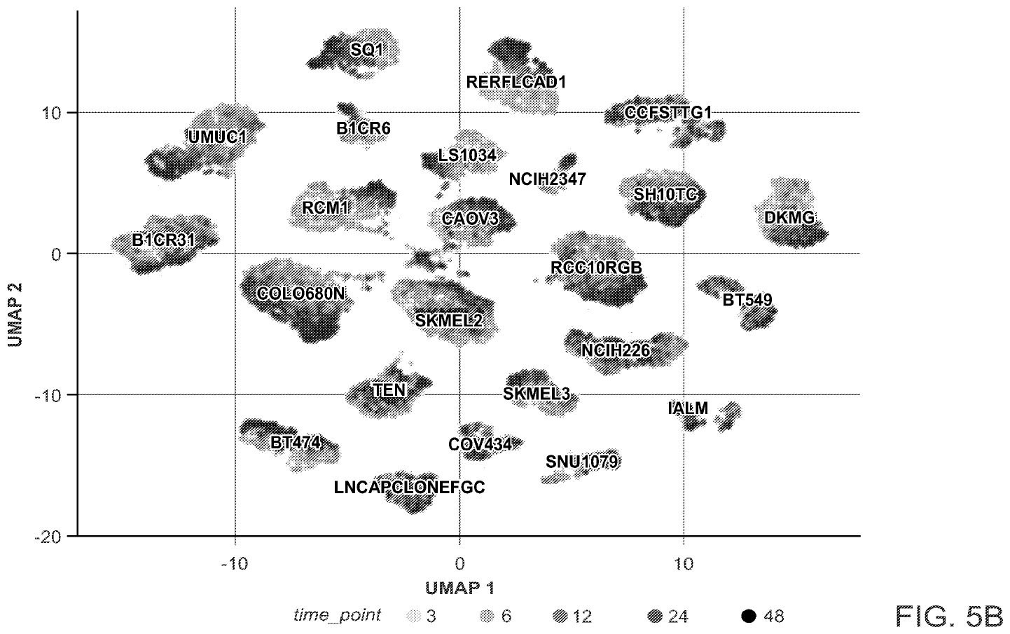

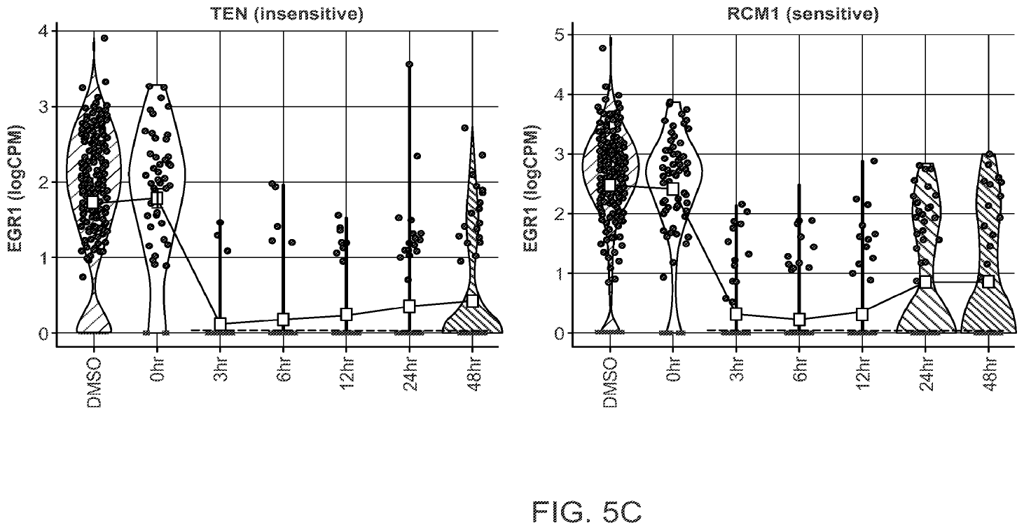

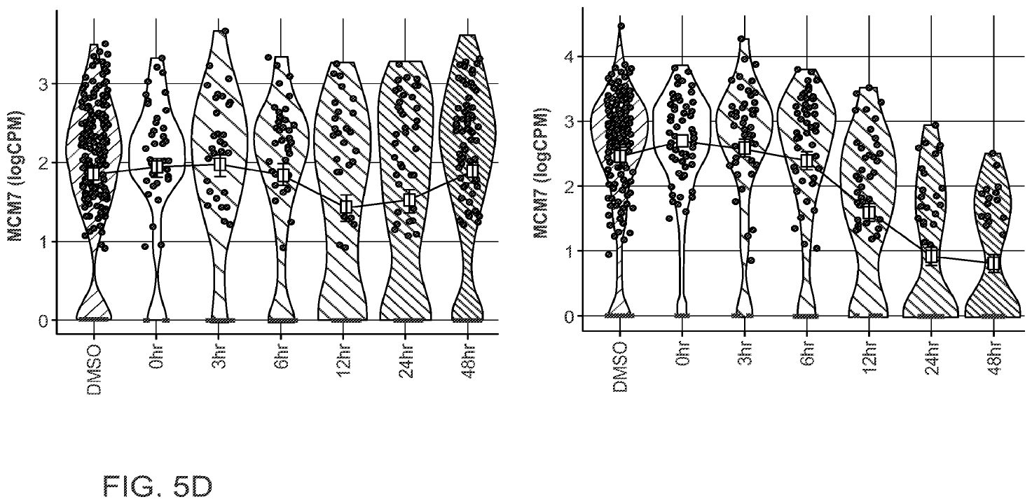

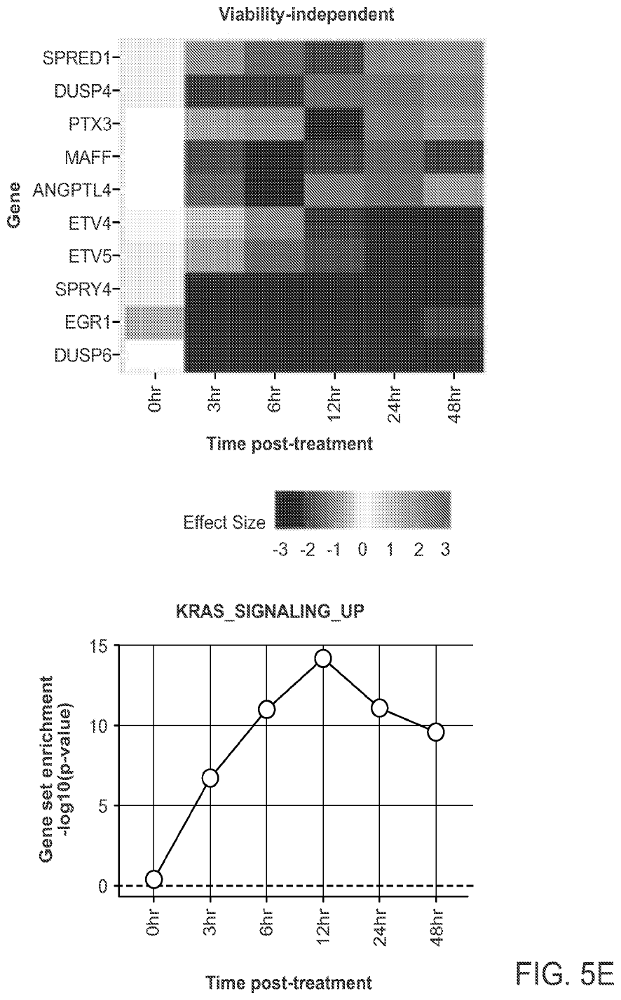

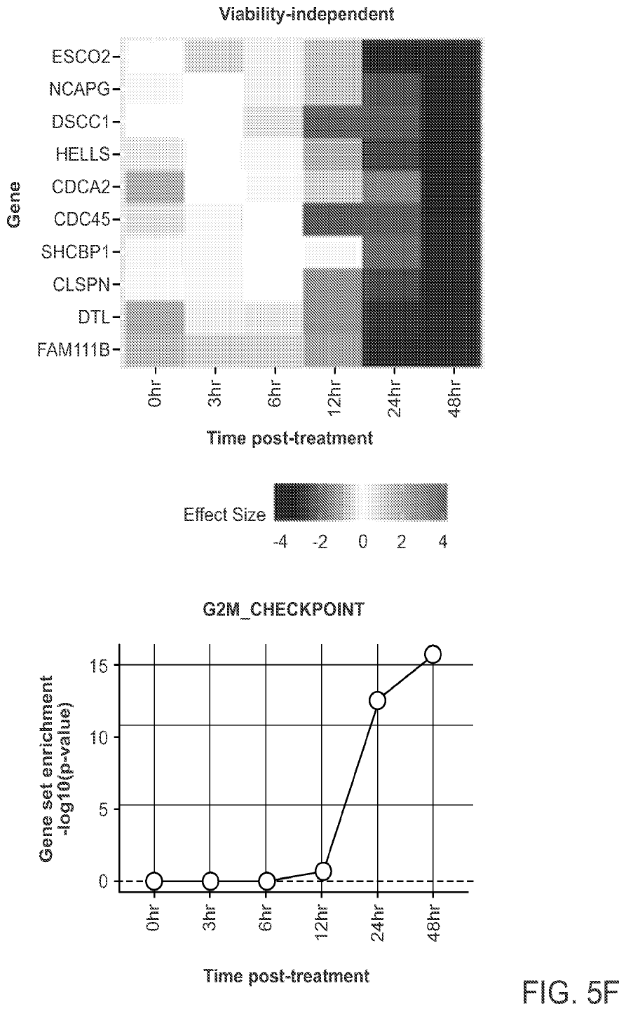

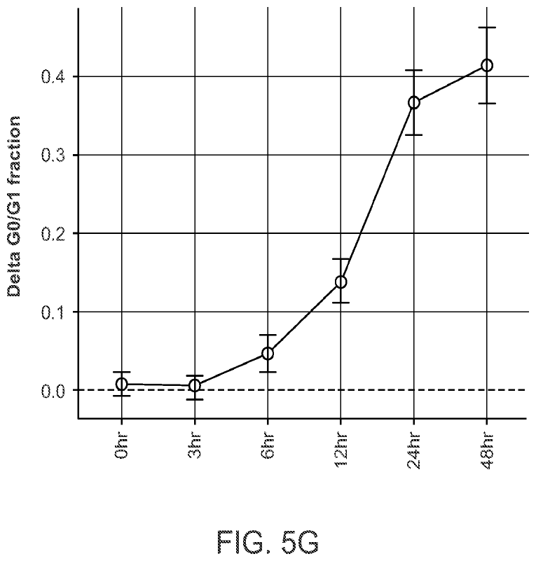

For FIGS. 5E and 5F, the time course of viability-independent and viability-related responses were plotted for the top 10 down-regulated genes in each component, taking the coefficient with the largest magnitude across post-treatment time points for each gene (after filtering for coefficients with FDR<0.1).

### Bortezomib Heterogeneity Analysis

To identify subpopulations of bortezomib treated cells (FIGS. 4D-4F), Seurat's default methods were used to normalize the data, detect variable genes, and compute PCs (using 5000 most variable genes, and 50 PCs). Seurat's default clustering methods were then used to cluster cells for each cell line (using the 20 nearest neighbors, and a clustering resolution parameter of 0.25).

### Trametinib Heterogeneity Analysis

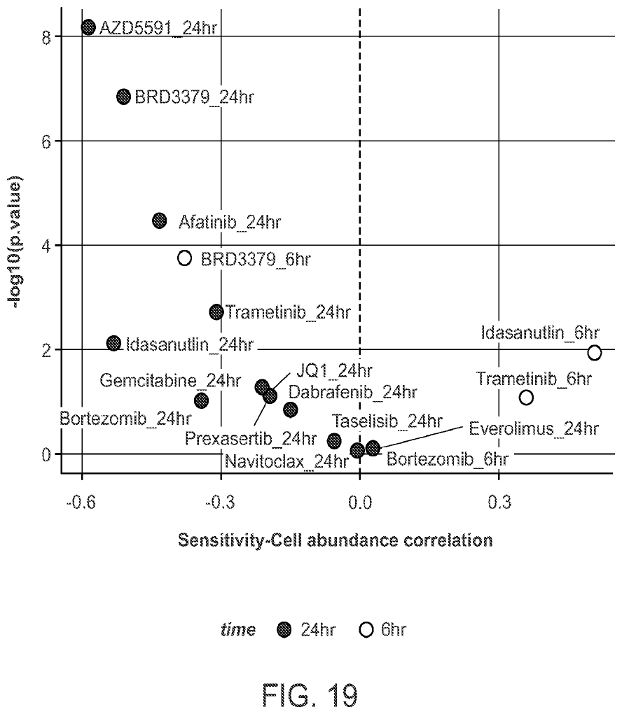

To identify sub-populations of cells from a given cell line in a consistent fashion across baseline and treatment conditions (for analysis and presentation in FIG. 19), the following procedure was employed. After restricting to cells from the target cell line, the scTransform method (Hafemeiester et al. 2019) was used to normalize the data, identify 5,000 variable genes, and regress out experimental condition as a covariate to align clusters across conditions. Seurat's default clustering methods (using the top 10 PCs, 20 nearest neighbors, and a clustering resolution parameter of 0.25) were then used to identify clusters jointly across treated and control cells.

### GPX4 KO Data

Differential expression analysis of GPX4 KO was done by comparing the average effects of the two GPX4 targeting guides against the average of the two control guides (one targeting and one non-targeting), following the same analysis procedure as used for drug treatment data. GPX4 dependent and non-dependent lines were identified using the estimated probability of GPX4 dependency for each cell line from the Achilles 19Q3 “gene dependency” file (Broad DepMap 2019). Cell lines with GPX4 dependency probability greater than 0.5 were considered dependent.

### L1000 Comparison

L1000 gene expression signatures were taken from either the LINCS Phase 2 data (GEO accession GSE70138, downloaded from www.amp.pharm.mssm.edu/Slicr) or LINCS Phase 1 data (GEO accession GSE92742, downloaded from www.clue.io). Phase 2 data were used when available (for the compounds trametinib, everolimus, and JQ1), while Phase 1 data were used for the remaining compounds (bortezomib, gemcitabine, and navitoclax). Comparisons were made using the average of the L1000 Level 5 gene expression signatures across all samples for a given drug, and the average log-fold change values across all cell lines from the MIX-Seq experiment for that drug (24 hours post-treatment).

### Example 2: Multiplexed Cell Line Transcriptional Profiling Using scRNA-Seq

Development of the MIX-seq platform is disclosed herein. MIX-seq uses single-cell RNA sequencing (scRNA-seq) to measure the transcriptional effects of a perturbation across diverse cancer cell lines cultured and perturbed in one or more pools (see FIG. 1A). These pools were then treated with a small molecule compound (or genetic perturbation) (Yu et al. 2016). To ascertain transcriptional response signatures, cell-specific transcriptomes were measured using scRNA-seq after a defined time interval following perturbation. To assign each profiled cell to its respective cell line, an optimized computational demultiplexing method was developed that classified cells by their genetic fingerprints, similar to recently developed methods, such as the Demuxlet method.

Specifically, for each single cell, the reference cell line whose genotype across a panel of commonly occurring SNPs would most likely explain the observed pattern of mRNA SNP reads was estimated (FIG. 1B). As previously demonstrated, this also allows for identification of multiplets of co-encapsulated cells (Kang et al. 2018), where two or more cells from different cell lines were unintentionally tagged with the same cell barcode during droplet-based single-cell library preparation. The pipeline developed by the instant disclosure utilized a fast approximation strategy to identify such ‘doublets’ that efficiently scaled to pools of hundreds of cell lines (see Example 1 above). It also provided quality metrics that were used to identify and remove low-quality cells (FIGS. 6A-6C), such as empty droplets (Macosko et al. 2015 and Stoeckius et al. 2018).

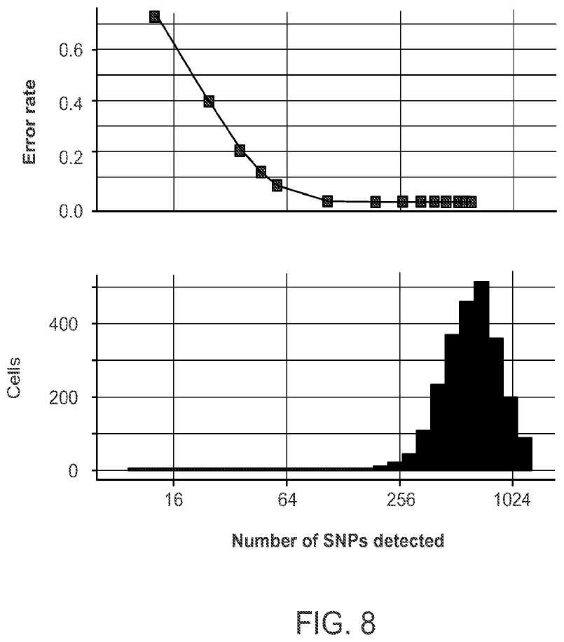

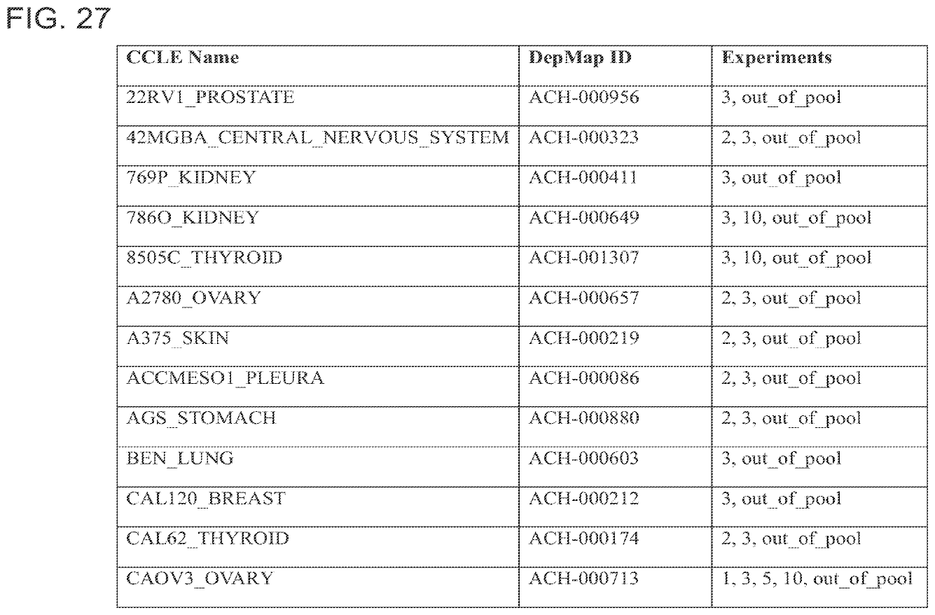

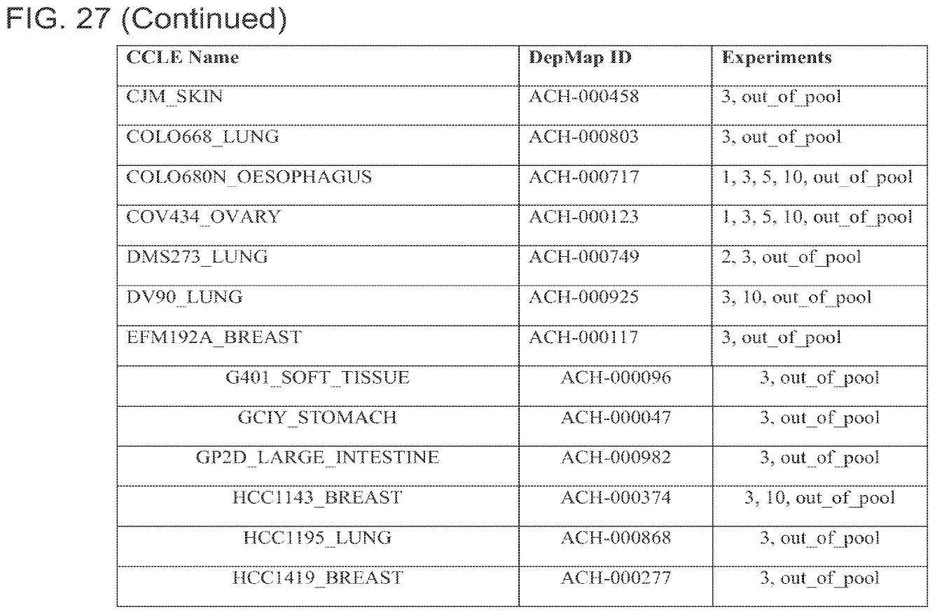

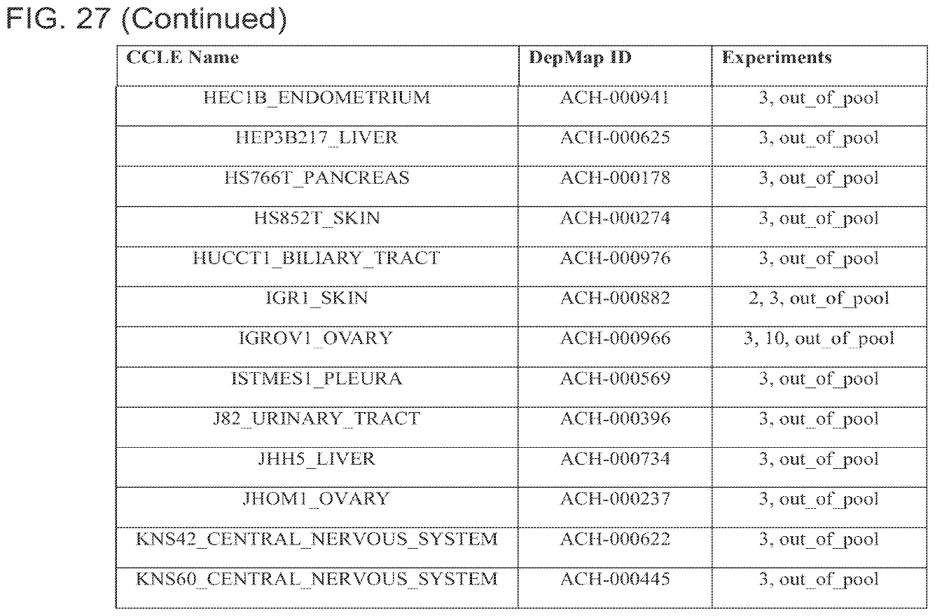

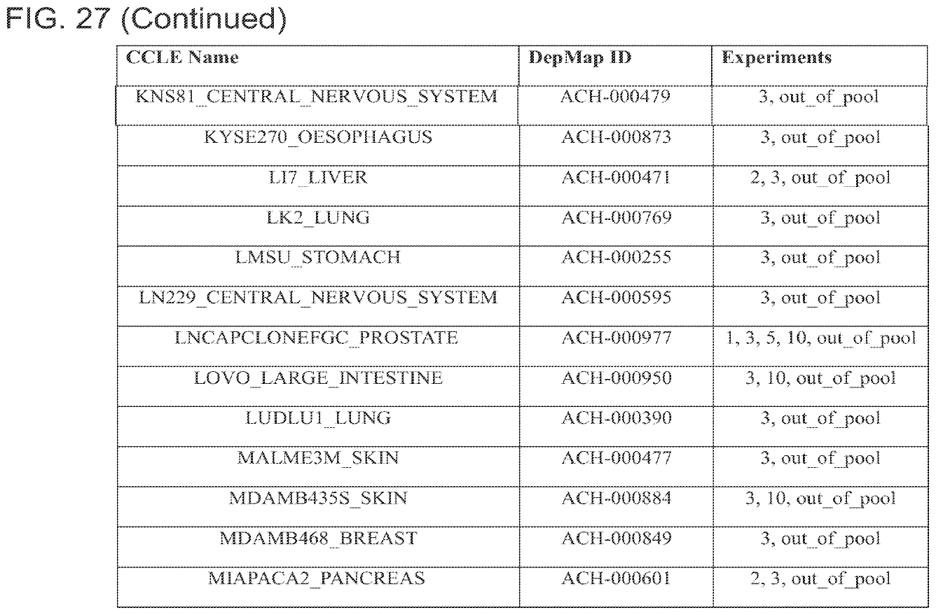

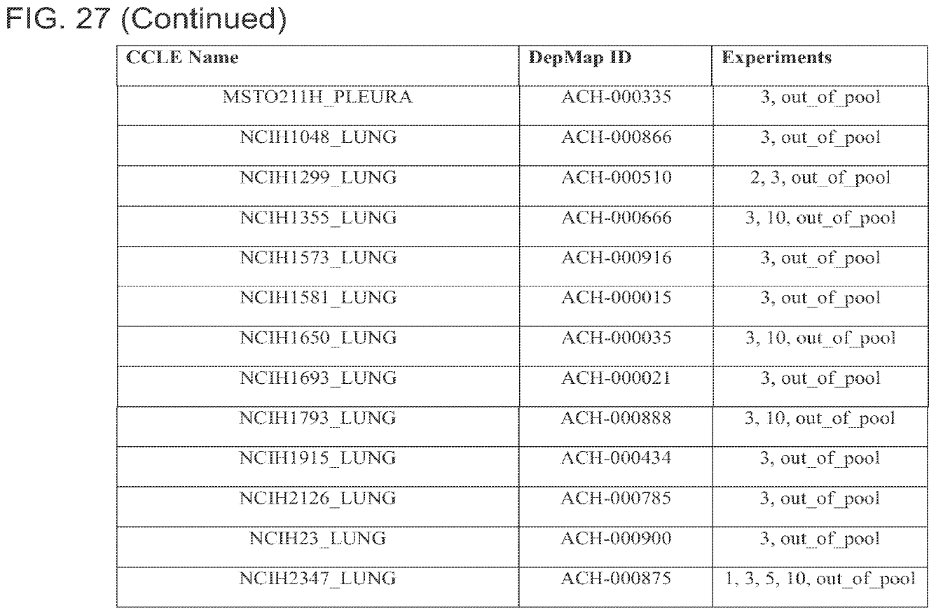

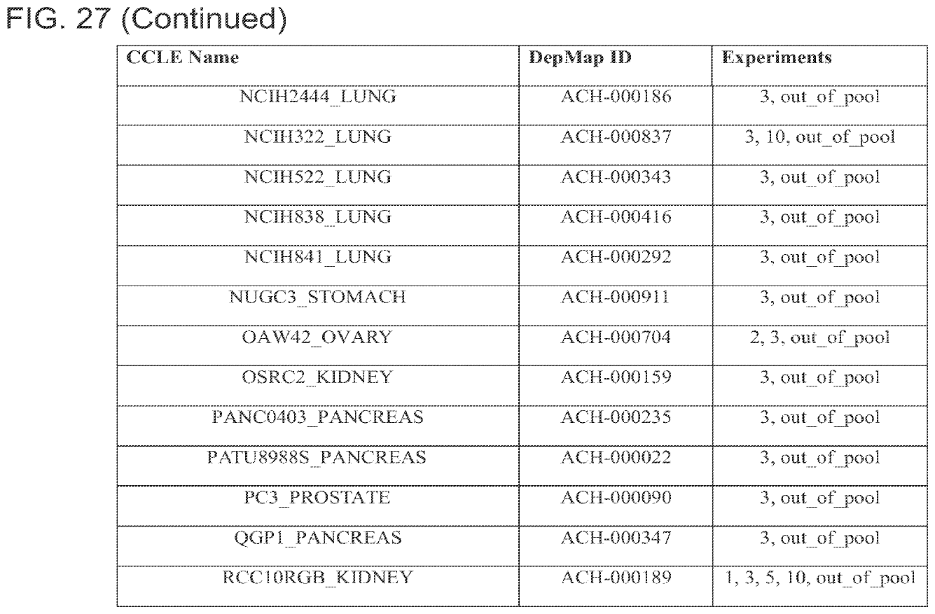

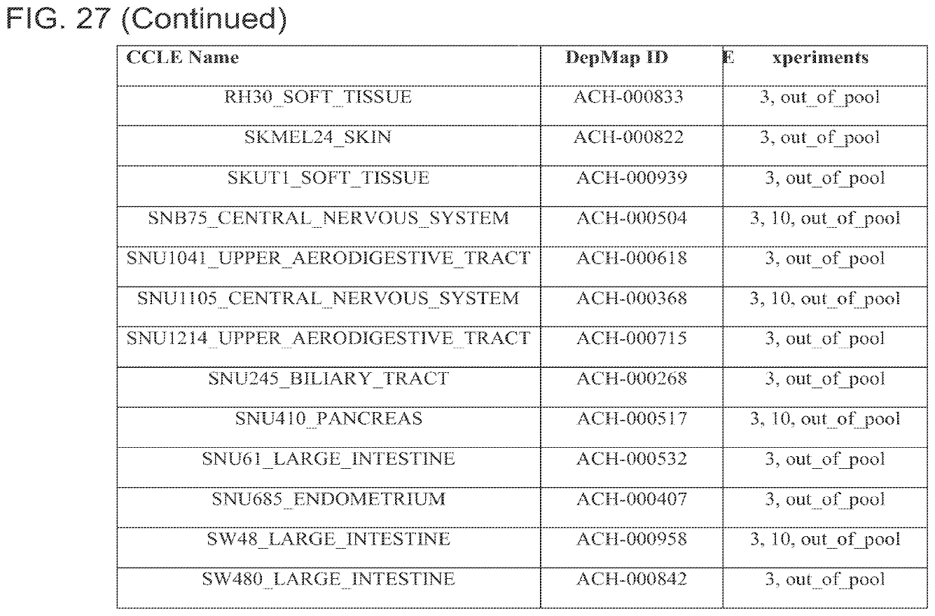

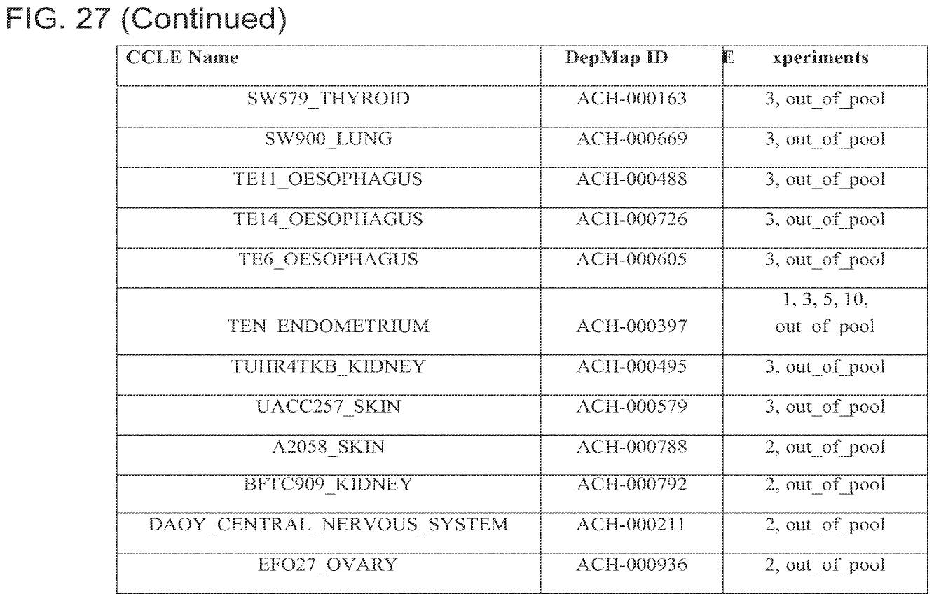

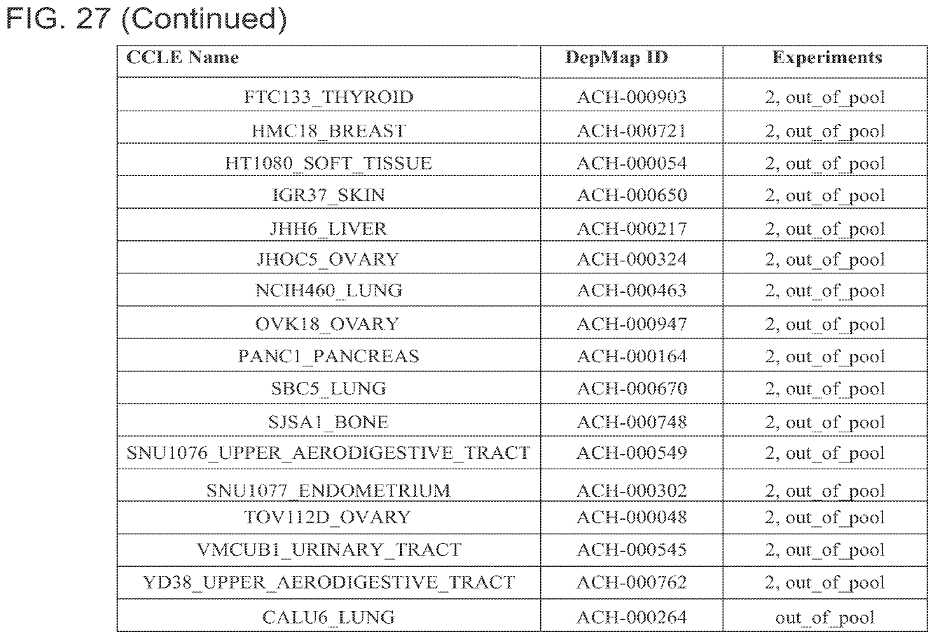

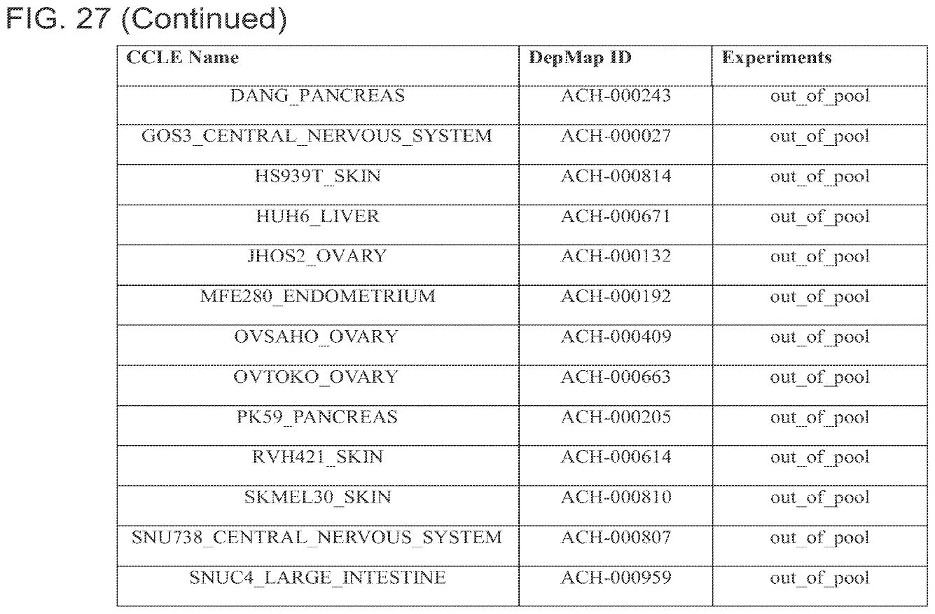

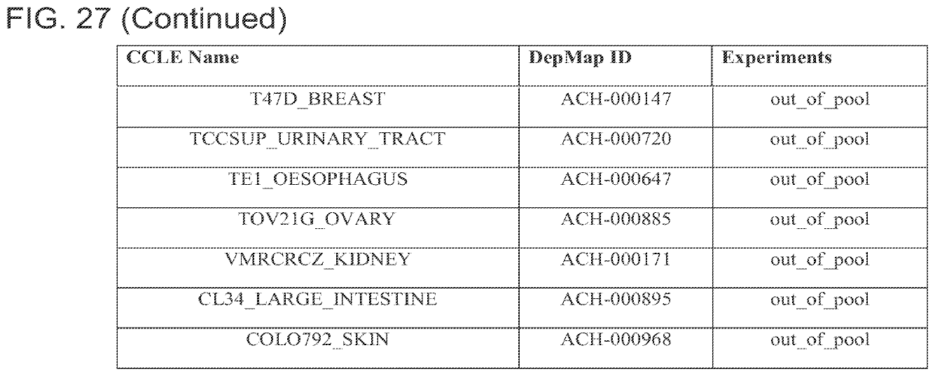

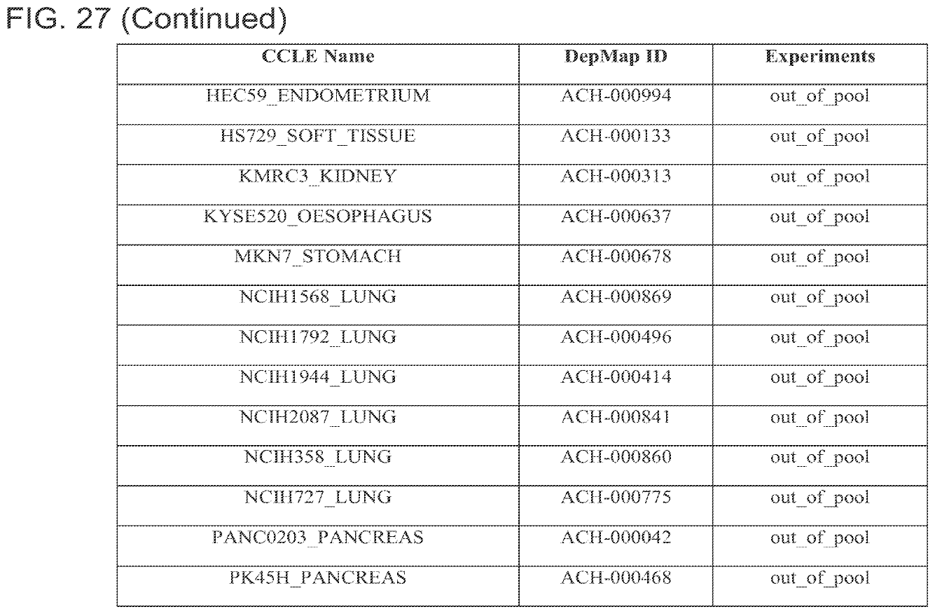

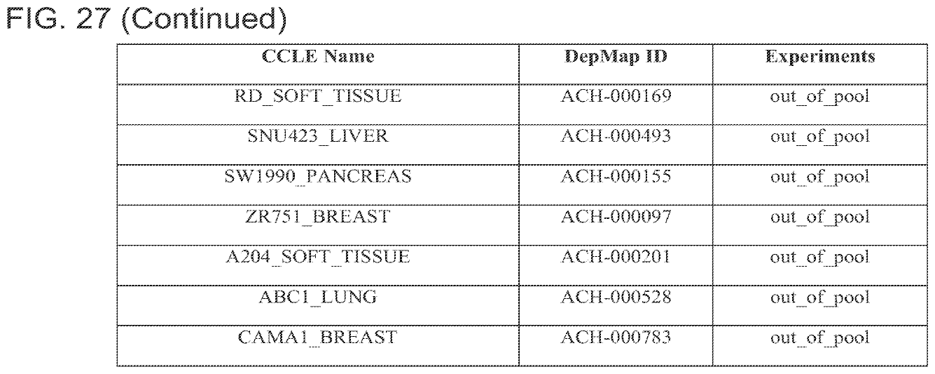

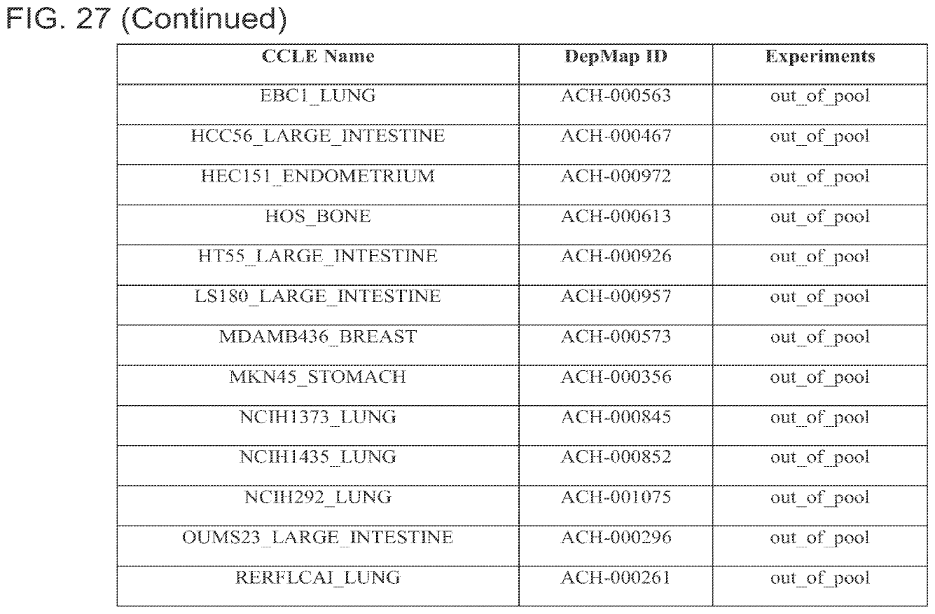

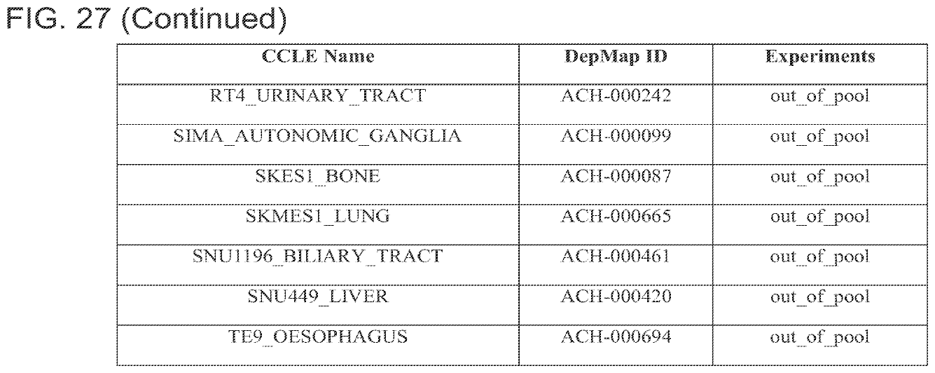

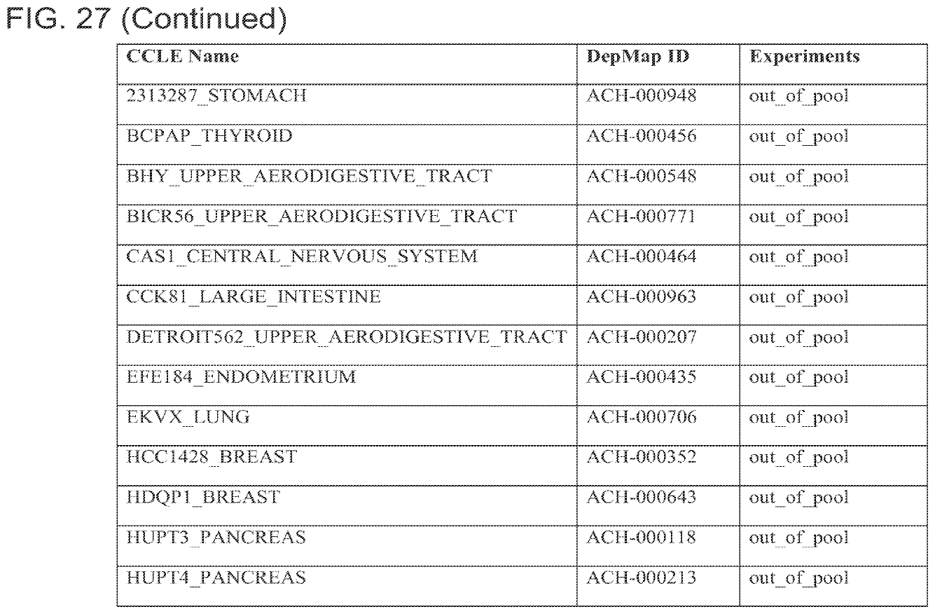

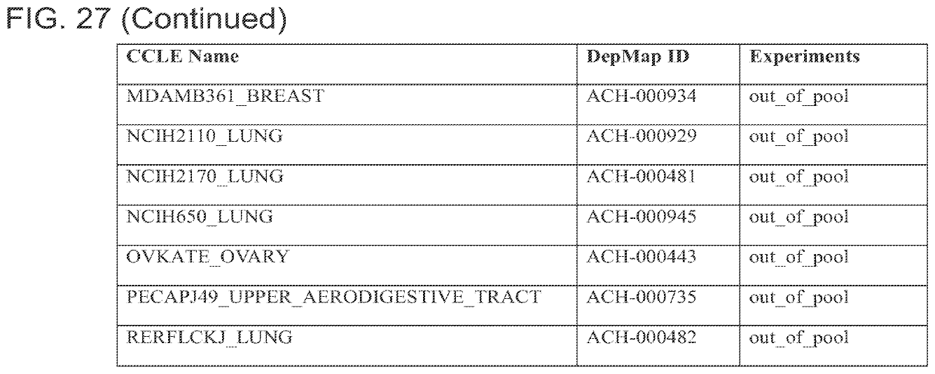

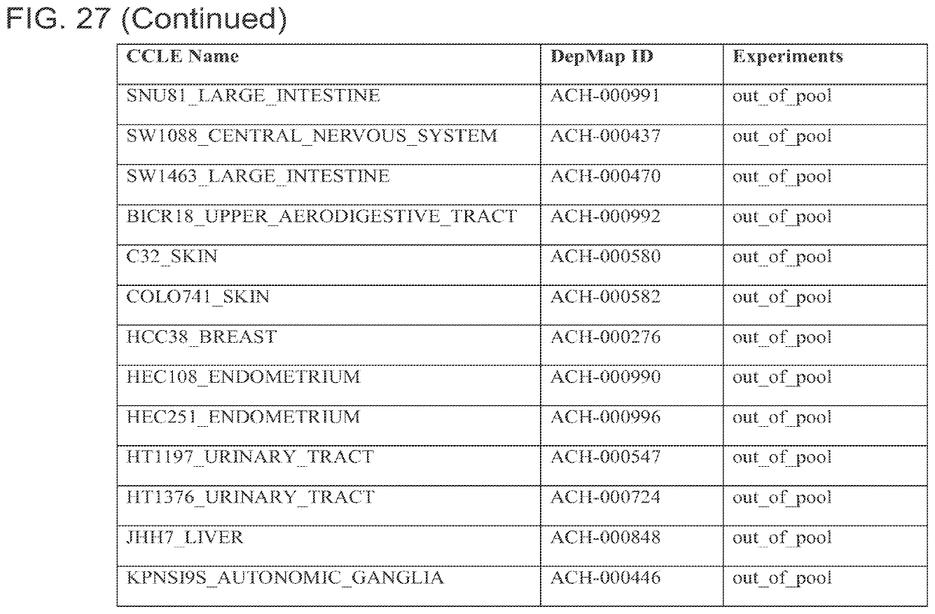

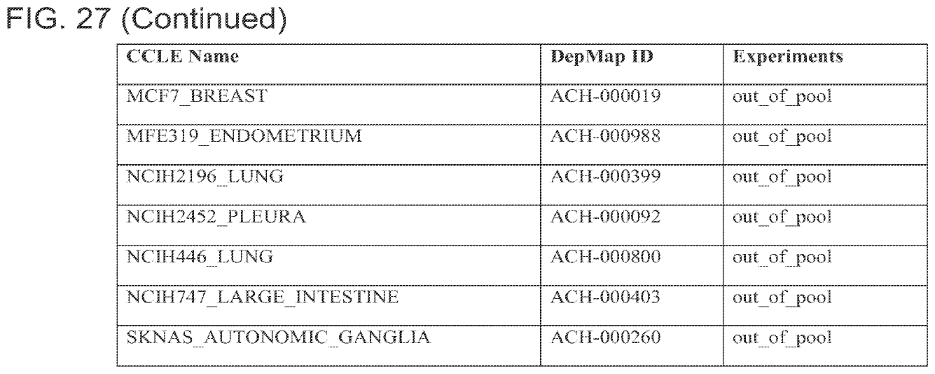

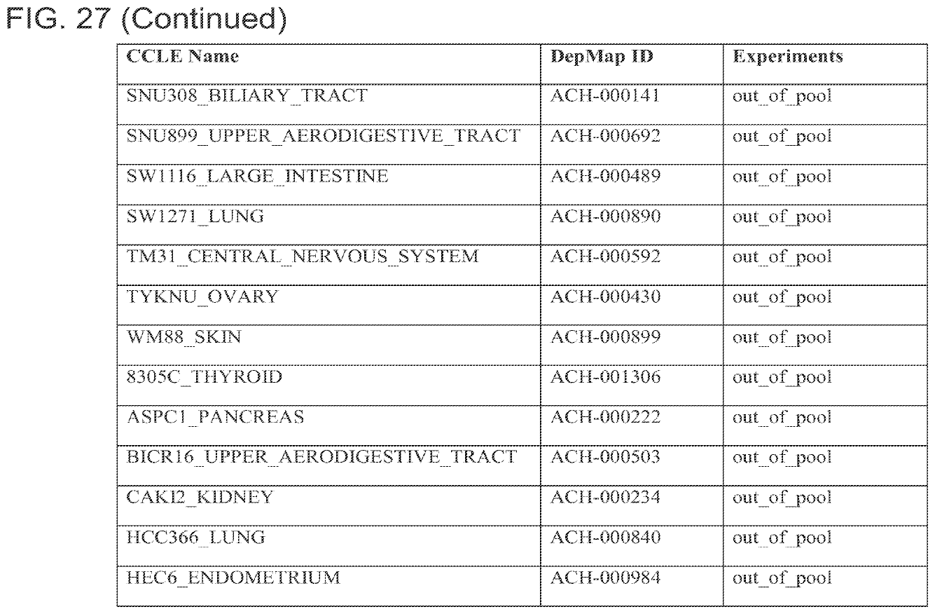

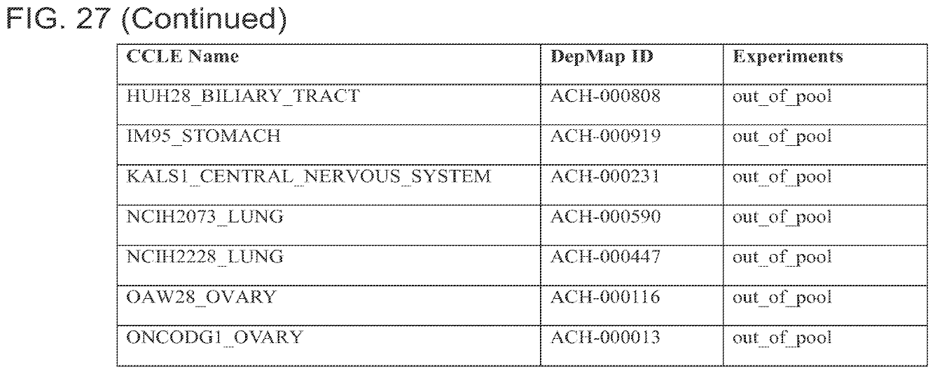

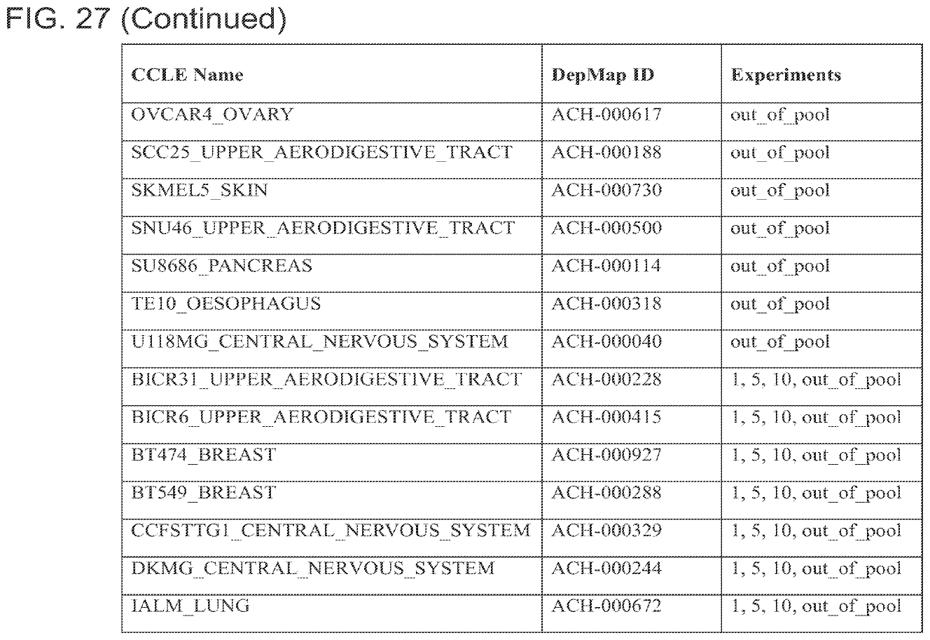

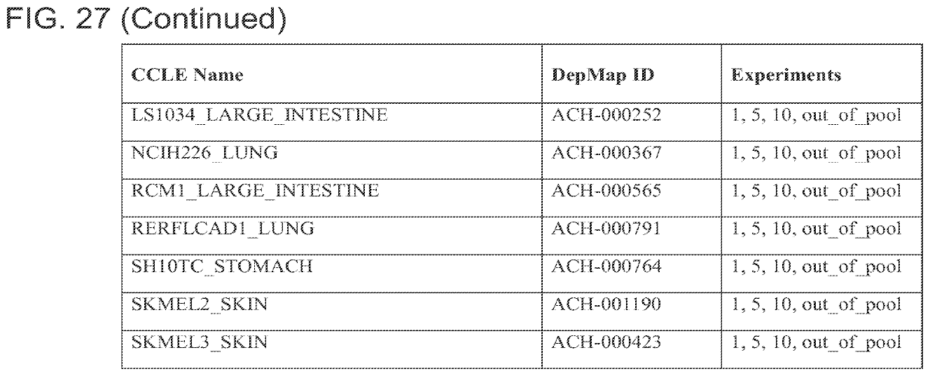

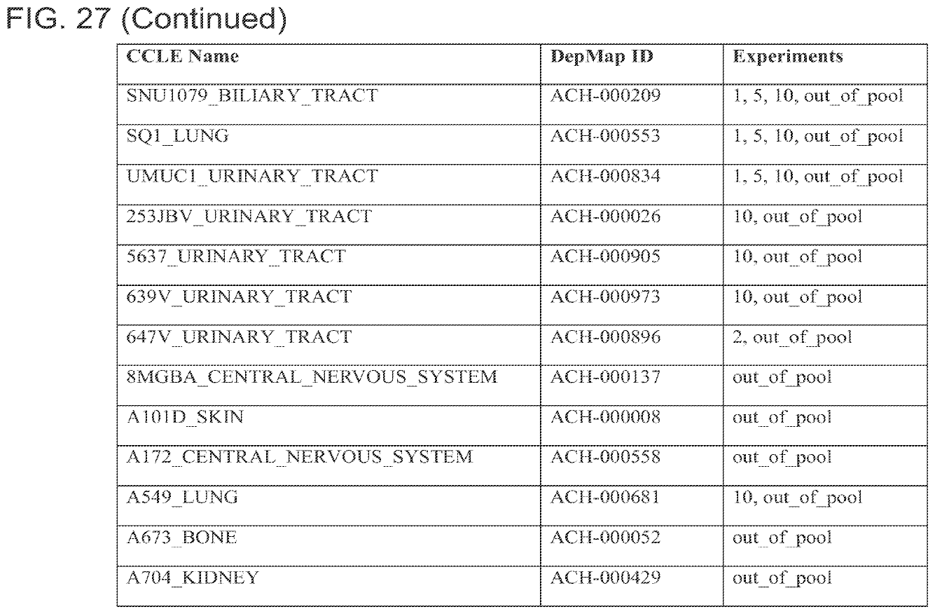

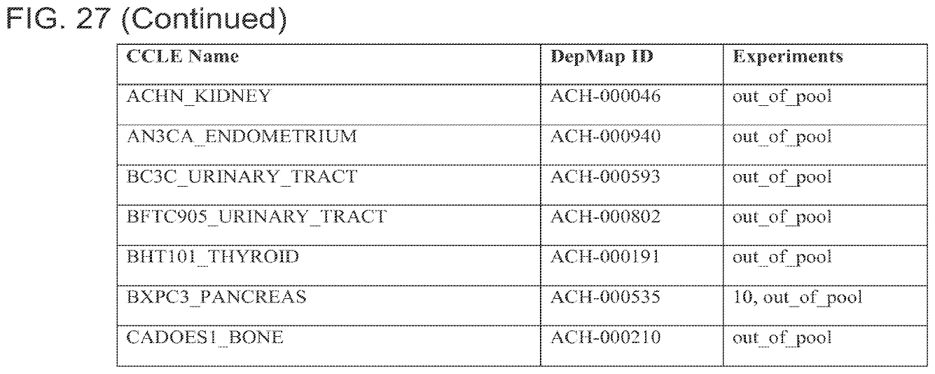

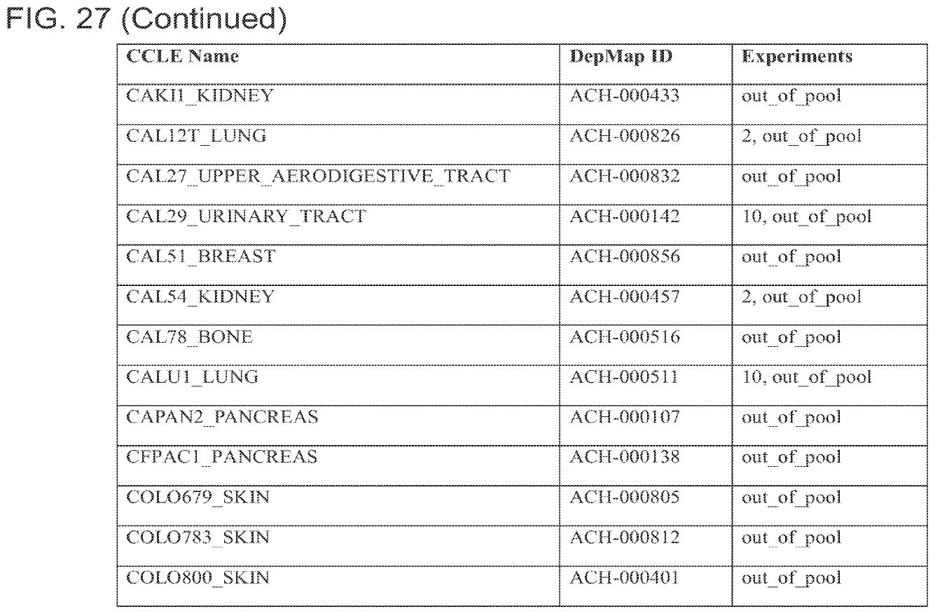

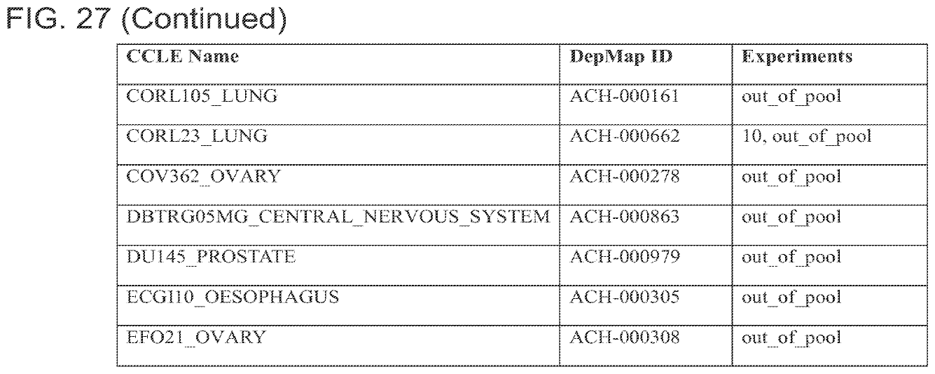

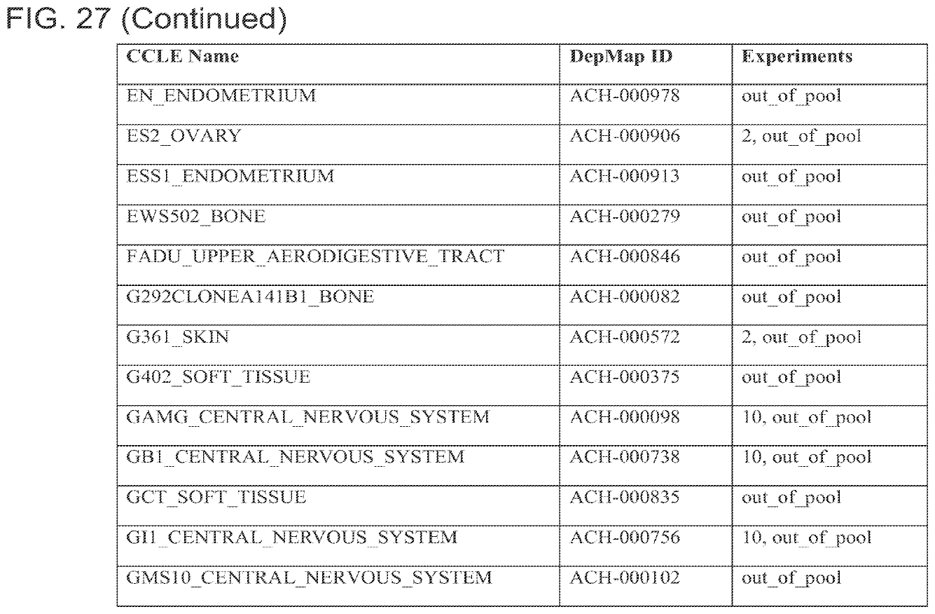

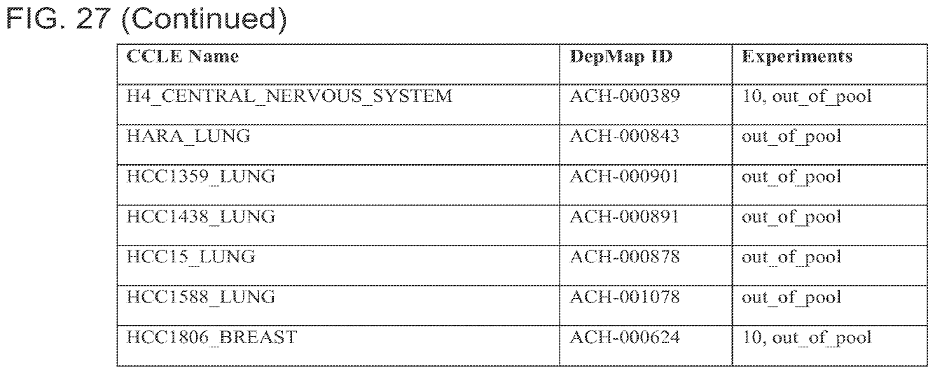

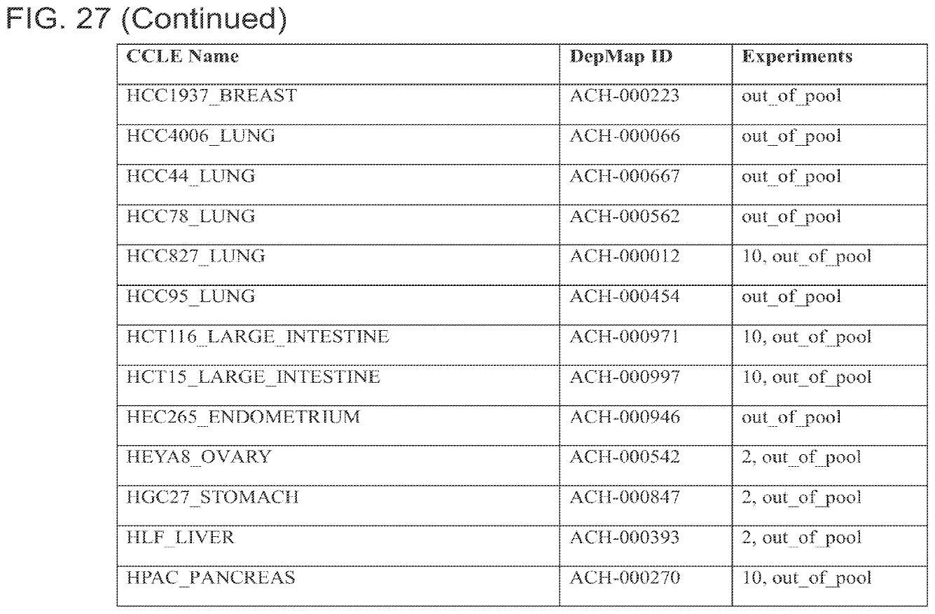

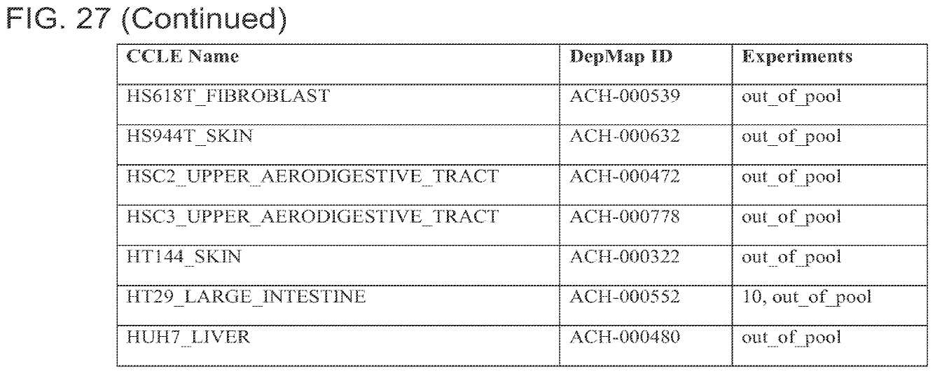

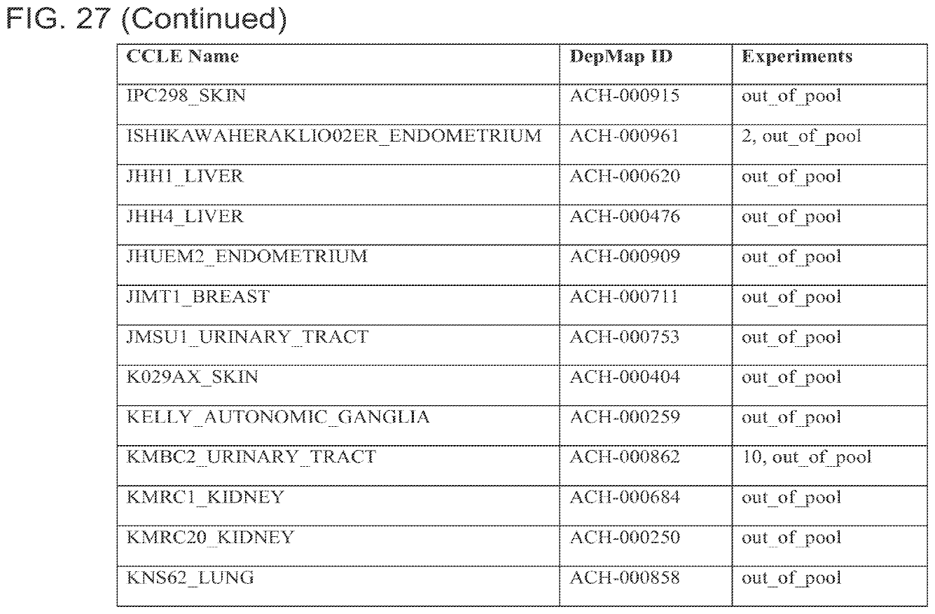

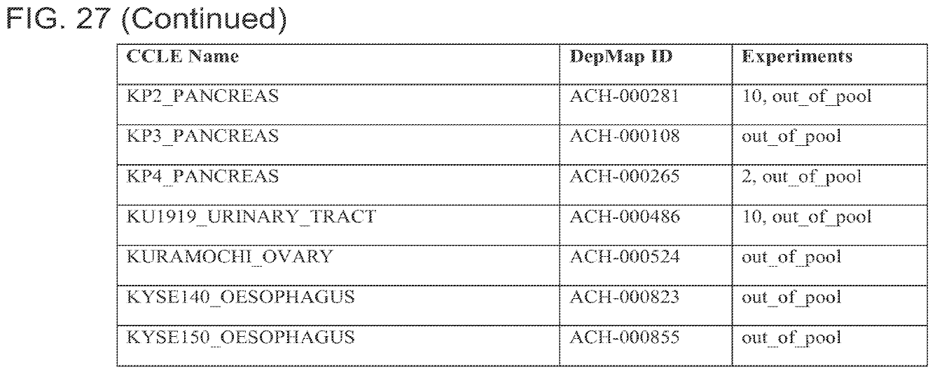

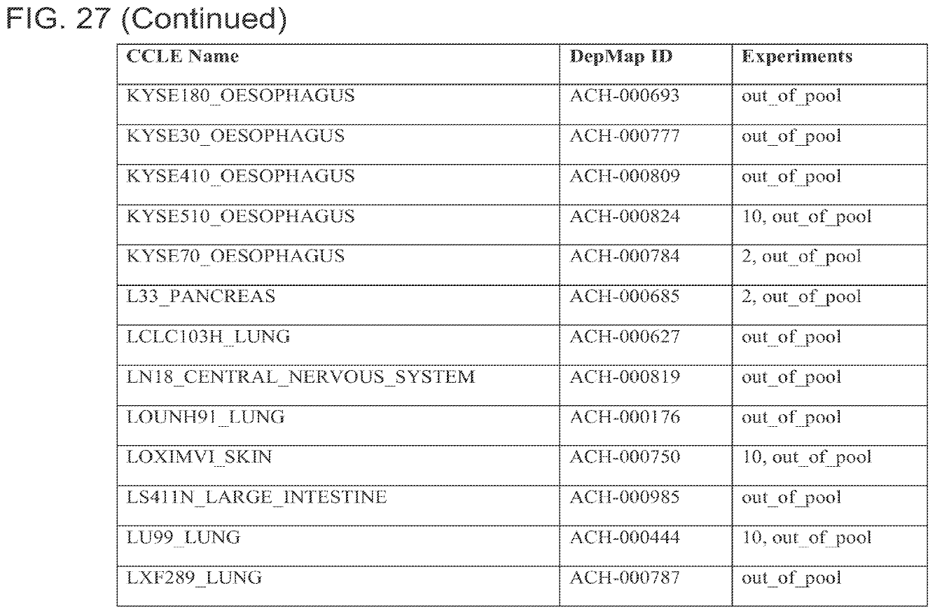

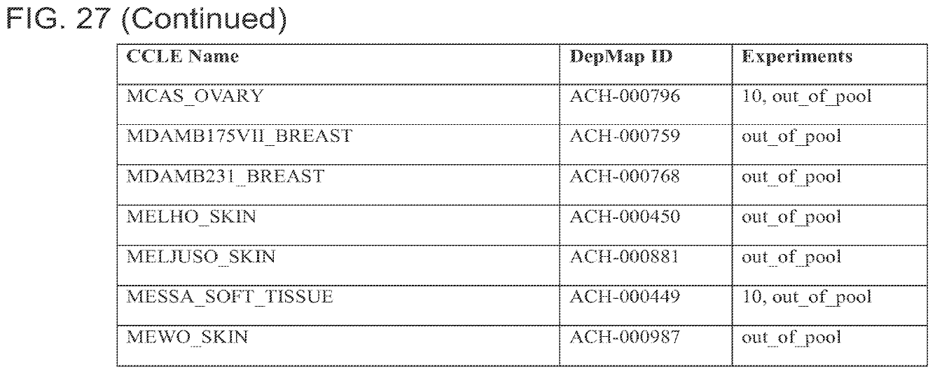

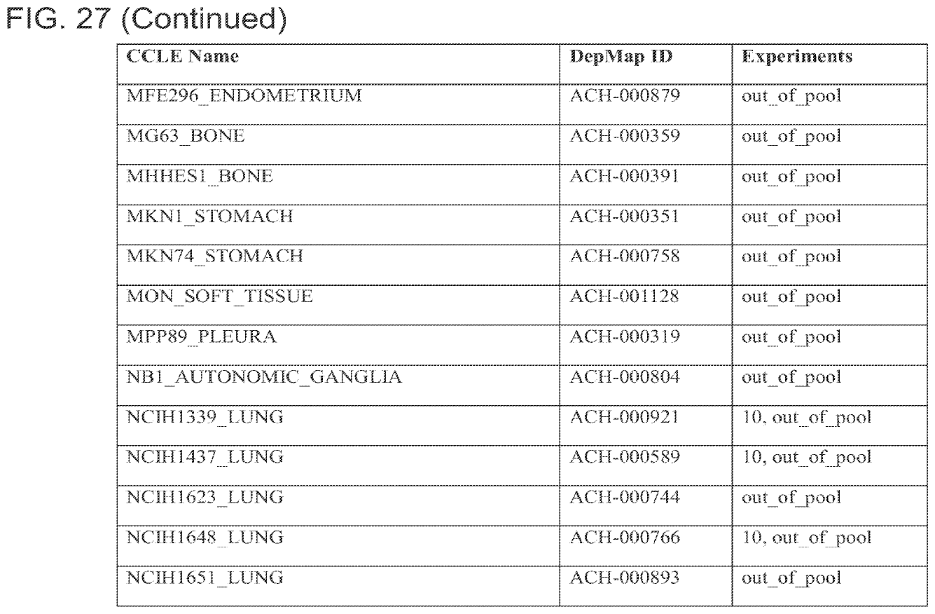

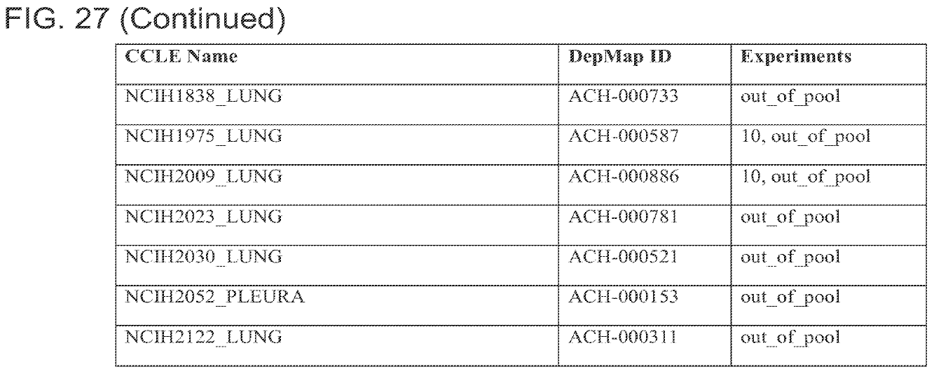

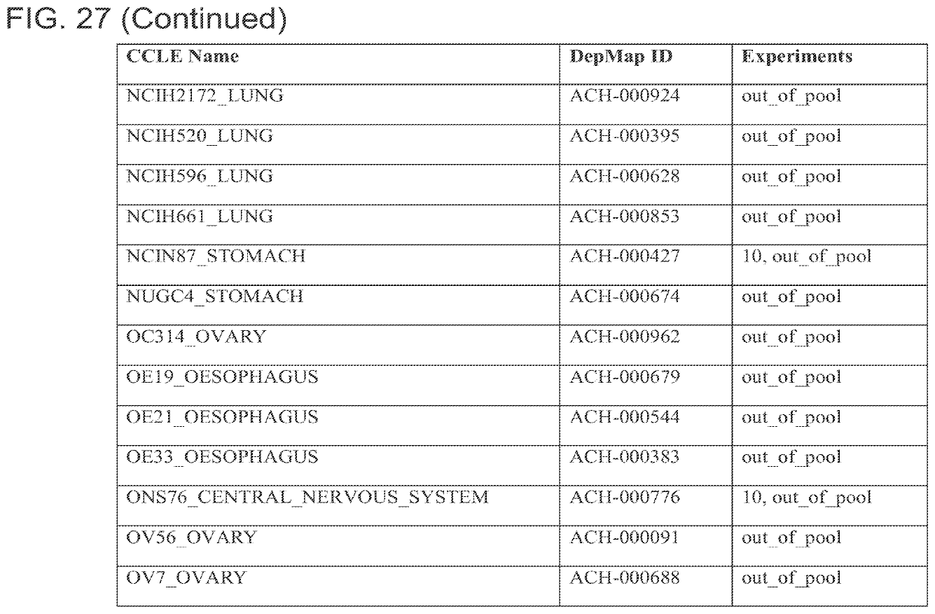

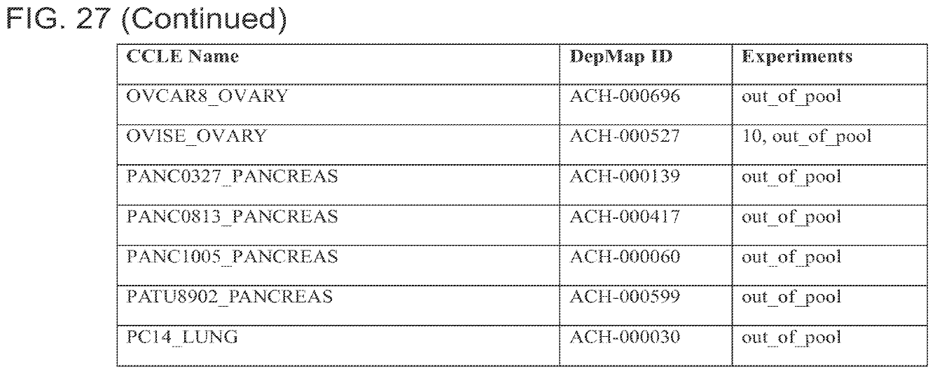

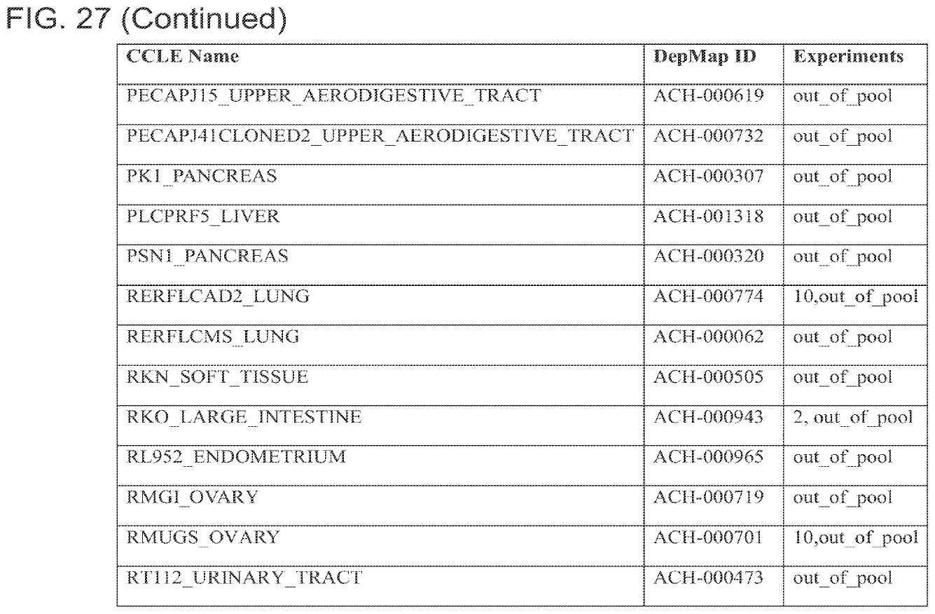

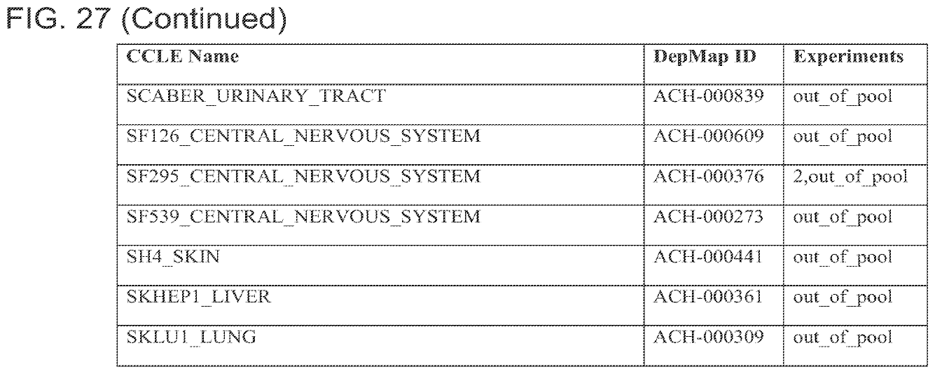

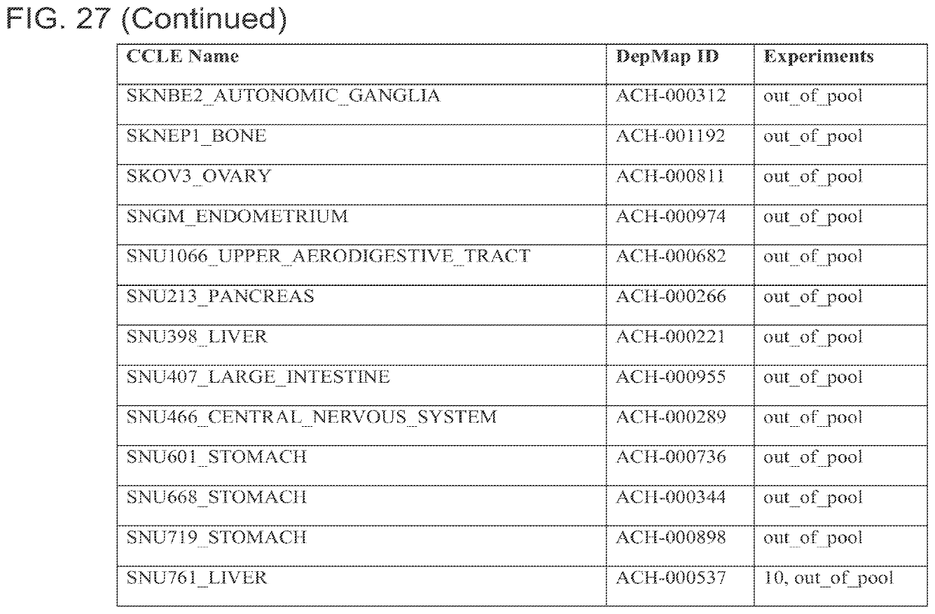

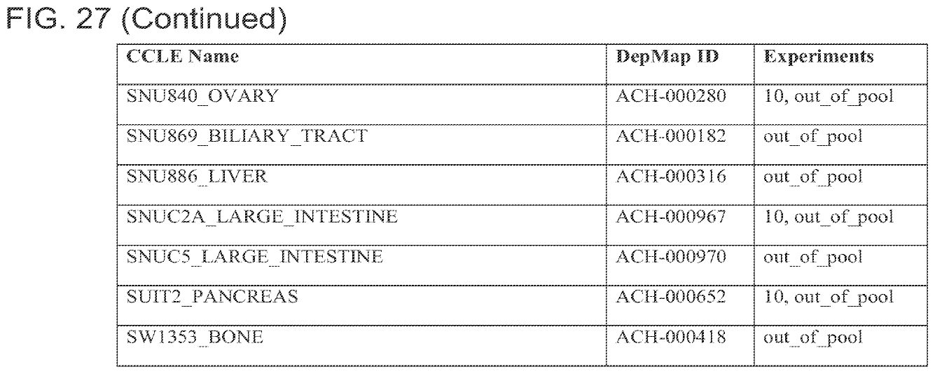

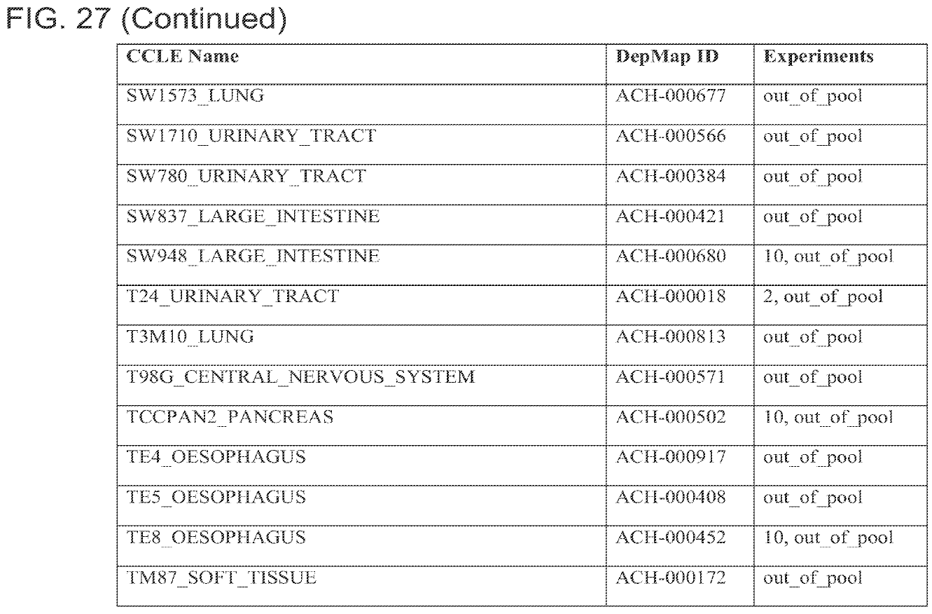

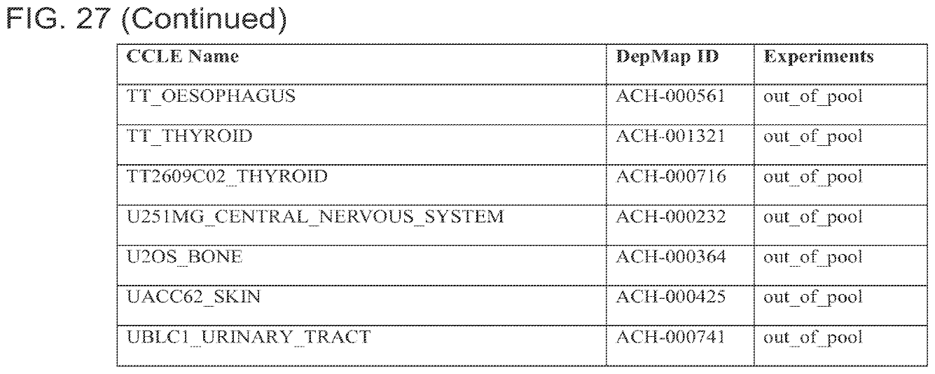

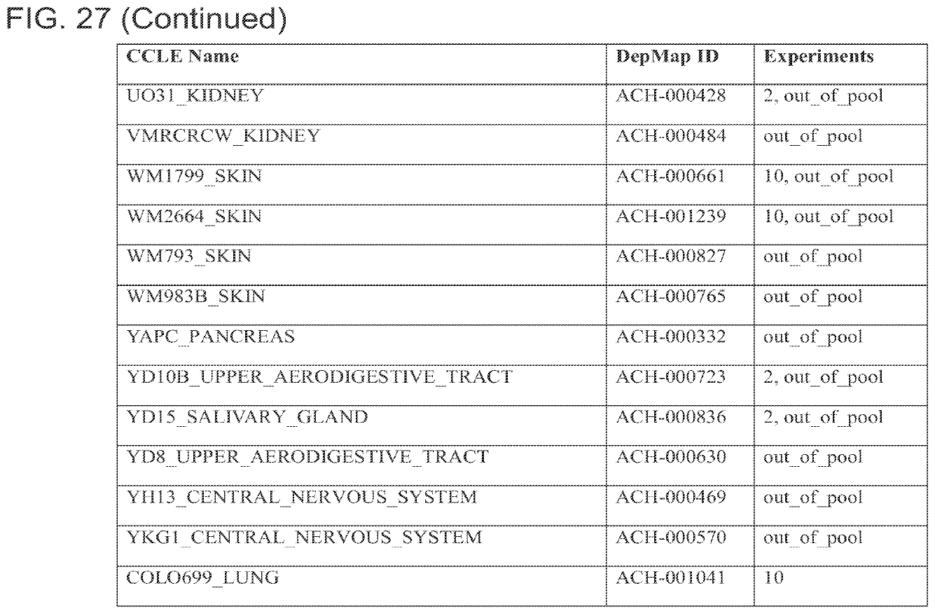

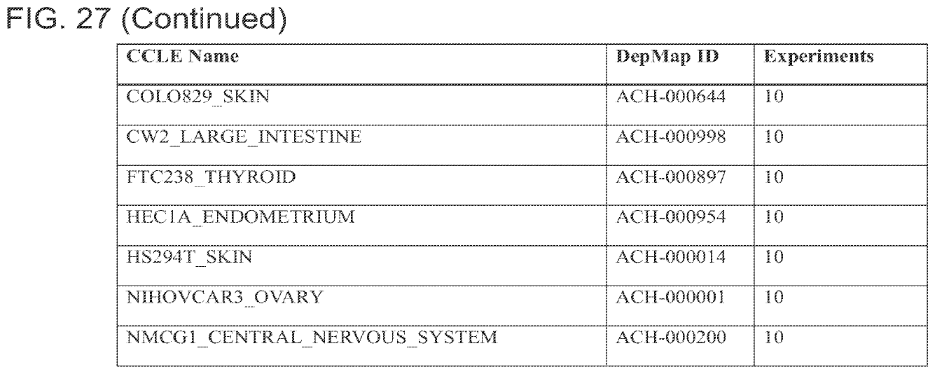

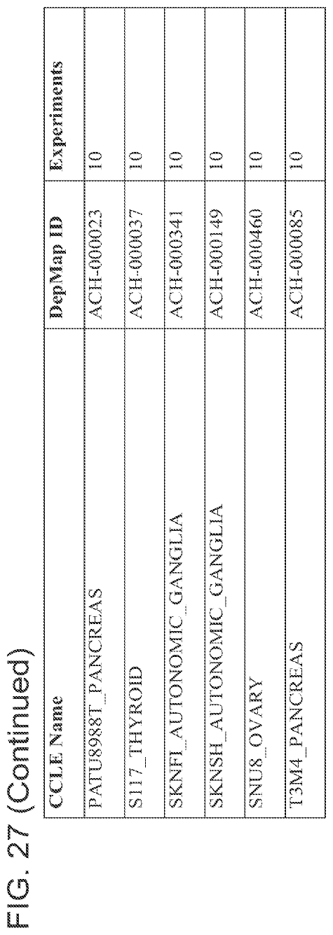

The classification accuracy of the SNP-based demultiplexing was confirmed in two ways. First, cell identities were classified based on either their gene expression or SNP profiles (see Example 1 above). Notably, these independent classifications were in excellent (>99%) agreement (FIGS. 7A and 7B). While either feature can be used to accurately classify cell identities, SNP-based classification was employed herein by the methods of the instant disclosure, as it is inherently robust to perturbations that dramatically alter the cells' expression profiles. Further, SNP-based classification can be applied to pools of primary cells of the same type from different individuals (e.g., iPS cells). Second, the SNP classification model was allowed to select from a larger panel of 494 reference cell lines (FIG. 27) and assess the frequency with which it identified cell lines that were not in the experimental pools. The model never picked an out-of-pool cell line (0/84,869 cells passing QC). Notably, though MIX-Seq was tested with experimental pools of up to 99 cell lines, these analyses showed that SNP profiles can distinguish among much larger (>500) cell line pools. Furthermore, down-sampling analysis showed that SNP-based cell classifications can be applied robustly to cells with as few as 50-100 detected SNP sites (FIG. 8).

### Example 3: MIX-Seq Identified Selective Perturbation Responses and MOA

The ability of MIX-seq to distinguish biologically meaningful changes in gene expression in the context of drug treatment was evaluated. Pools of well-characterized cancer cell lines were treated with thirteen drugs, followed by subjecting the cells to scRNA-seq at 6 and/or 24 hours after treatment. These drugs included eight targeted cancer therapies with known mechanisms, four compounds that broadly kill most cell lines, and one tool compound (BRD-3379) with unknown mechanism of action (MoA) that was found to induce strong selective killing in a high-throughput screen. In all cases scRNA-seq based phenotyping was compared to long-term viability responses measured for these drugs and cell lines from the genomics of drug sensitivity in cancer (GDSC) dataset (Iorio et al. 2016 and Garnett et al. 2012), as well as data generated using the PRISM assay (Corsello et al. 2020 and Yu et al. 2016, see Example 1 above).

As a benchmark, nutlin, a selective MDM2 inhibitor, was first applied to a pool of 24 cell lines. MDM2 is a negative regulator of the tumor suppressor gene TP53, and nutlin is known to elicit rapid apoptosis and cell cycle arrest exclusively in cell lines that have wild-type (WT) TP53 (Vassilev et al. 2004). Jointly embedding the expression profiles of 7,317 single cells treated with either nutlin or vehicle control (DMSO) in 2D-space revealed clear clustering by cell line, with shifts in the nutlin-treated cell populations for some cell lines, but not others (FIG. 1C). Estimates of the average drug-induced changes in gene expression for each cell line (see Example 1) revealed a robust response in each of the 7 TP53 WT cell lines in the pool, but only minimal changes in cell lines harboring TP53 mutations, as expected (FIGS. 1D through 1F). Furthermore, gene set enrichment analysis (see Example 1 above) of the average transcriptional response among TP53 WT cell lines showed clear up-regulation of genes in the TP53 downstream pathway, as well as down-regulation of cell cycle processes (FIG. 1G).

Robust transcriptional response signatures were identified across nearly all thirteen drugs profiled. Further, these signatures were often highly informative about the compounds' MoA. For example, treatment with the proteasome inhibitor bortezomib elicited strong up-regulation of protein folding and heat-shock response pathways (FIG. 9A). The chemotherapy drug Gemcitabine altered expression of apoptosis-related genes (FIG. 9B), and mTOR signaling was the top down-regulated gene set following treatment with the mTOR inhibitor everolimus (FIG. 9C). Transcriptional response profiles measured using MIX-Seq showed good overall agreement with those measured by the L1000 gene expression assay (Subramanian et al. 2017) for the same compounds (FIG. 10). In conclusion, these results demonstrated the ability of MIX-Seq to measure selective transcriptional effects of a drug across a pool of cell lines, and highlighted the utility of such information for identifying a drug's cellular effects and MoA (Subramanian et al. 2017 and Corsello et al. 2020).

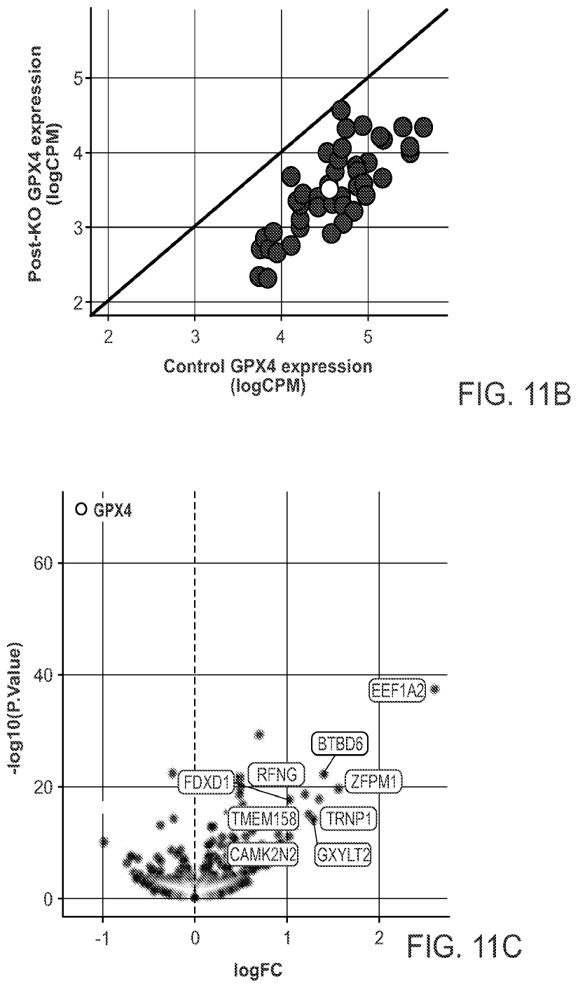

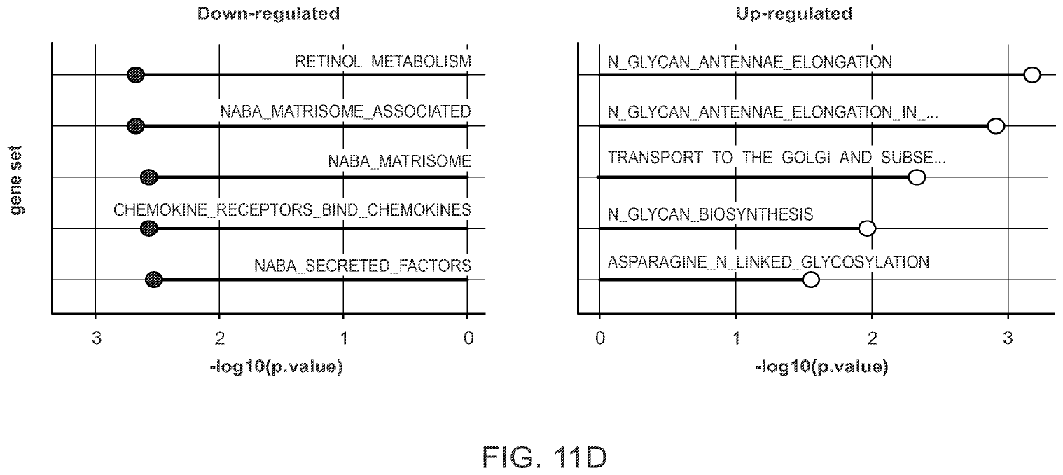

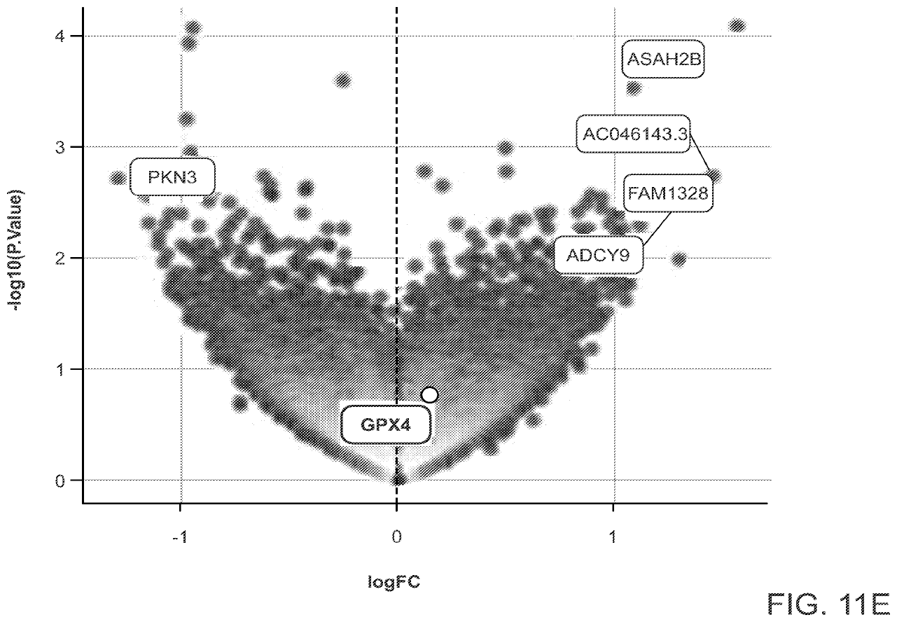

In addition to measuring drug-responses, MIX-Seq was also used to study the transcriptional effects of genetic perturbations in cell line pools. As a proof of concept, two sgRNAs targeting the gene glutathione peroxidase 4 (GPX4) were introduced by lentiviral transduction into a pool of 50 cell lines. scRNA-seq was then performed at either 72 or 96 hours post-infection (see Example 1 above). There was robust on-target reduction of GPX4 mRNA observed across all cell lines in the pool, and a transcriptional response consistent with the known function of GPX4 in lipid metabolism was identified (FIGS. 11A-11E).

### Example 4: Deconvolution of Viability-Related Response Signatures

A key advantage provided by profiling transcriptional responses across a large number of cell contexts using MIX-Seq is the observed ability for MIX-Seq to distinguish the overall transcriptional effects of a drug from the signature specifically associated with its viability effects. To accomplish this, a statistical modeling approach was employed relating the transcriptional changes measured in each cell line to their viability response in the drug sensitivity data from GDSC and PRISM (Iorio et al. 2016, Corsello et al. 2020, and Yu et al. 2016). Specifically, the change in expression of each gene was decomposed into two components: a viability-independent response component (β₀) characterizing the response of completely insensitive cell lines, and a viability-related response component (β₁) characterizing the difference between sensitive and insensitive cell lines (refer to Example 1 above).

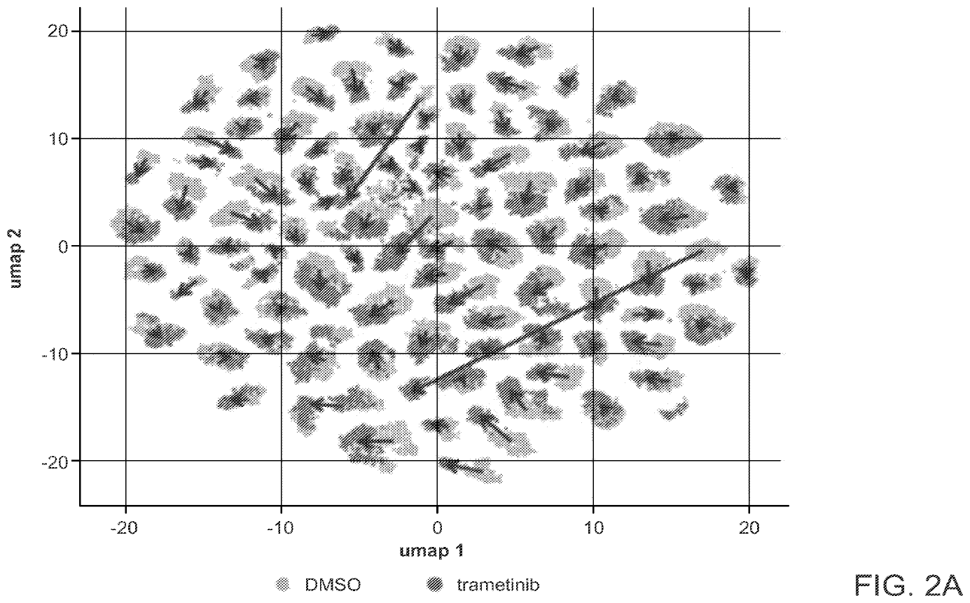

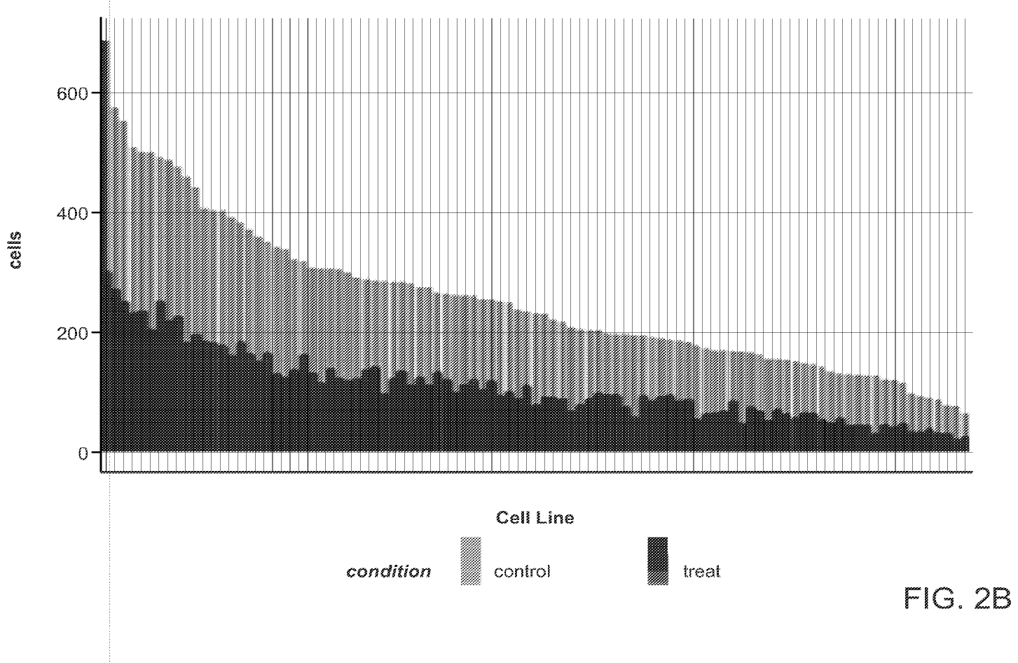

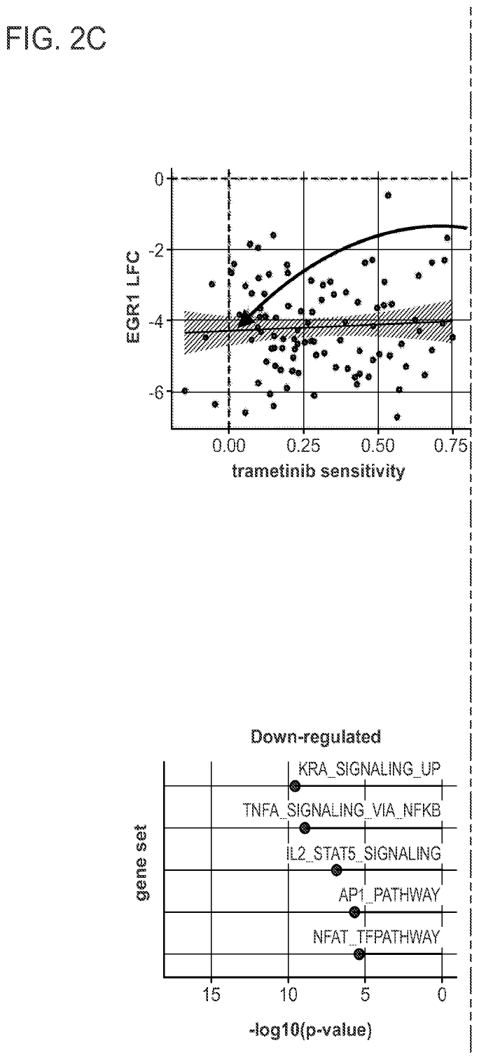

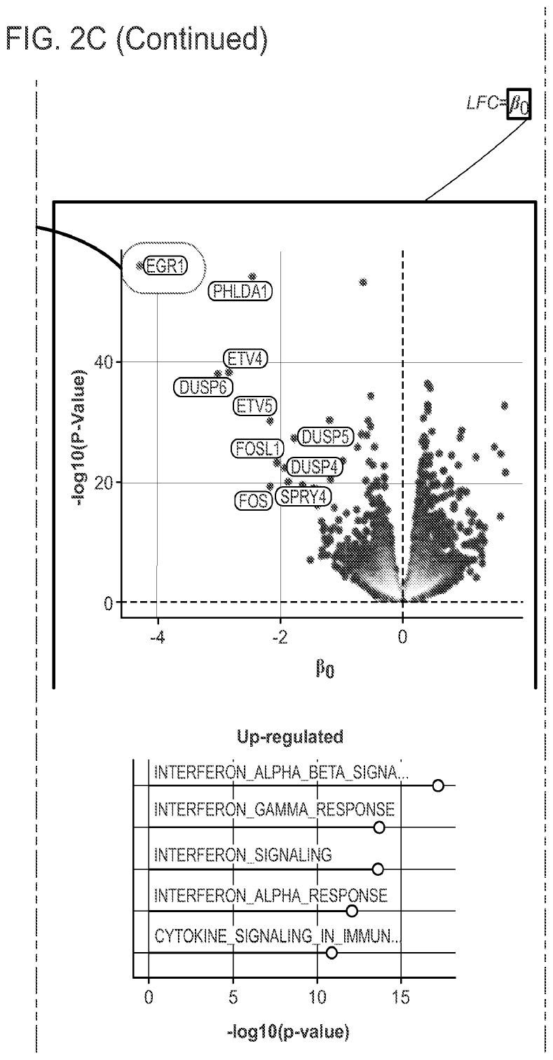

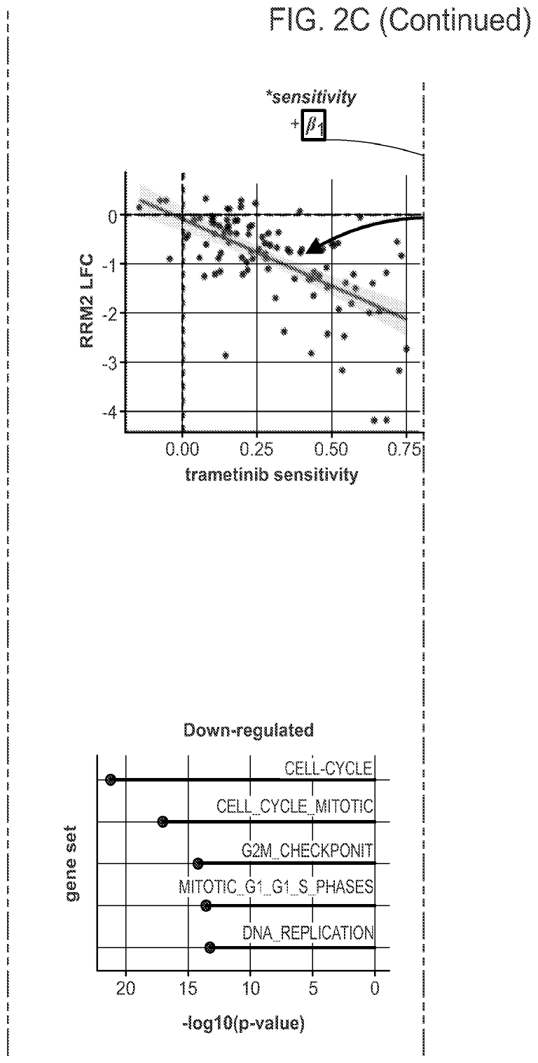

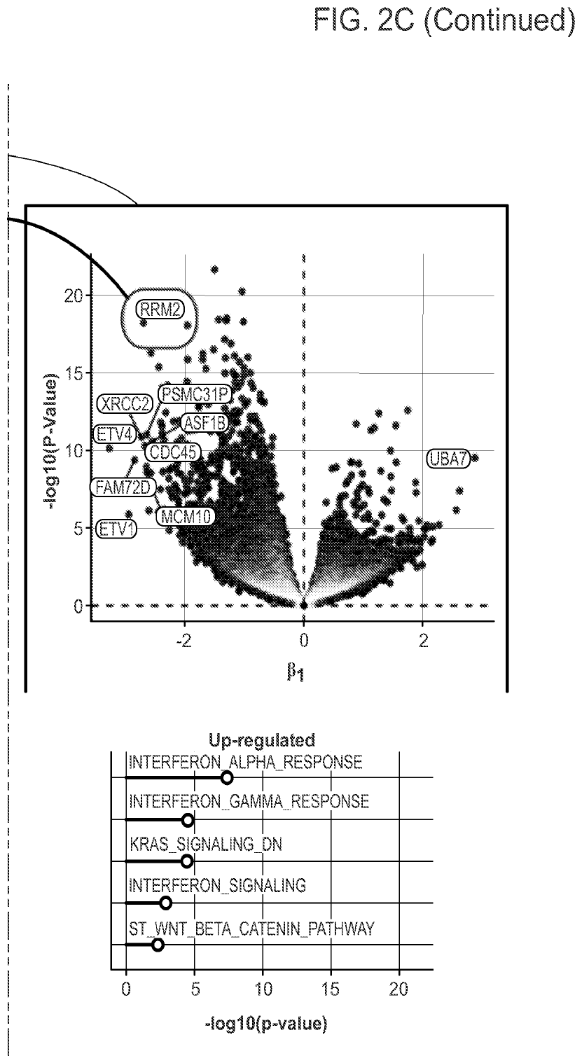

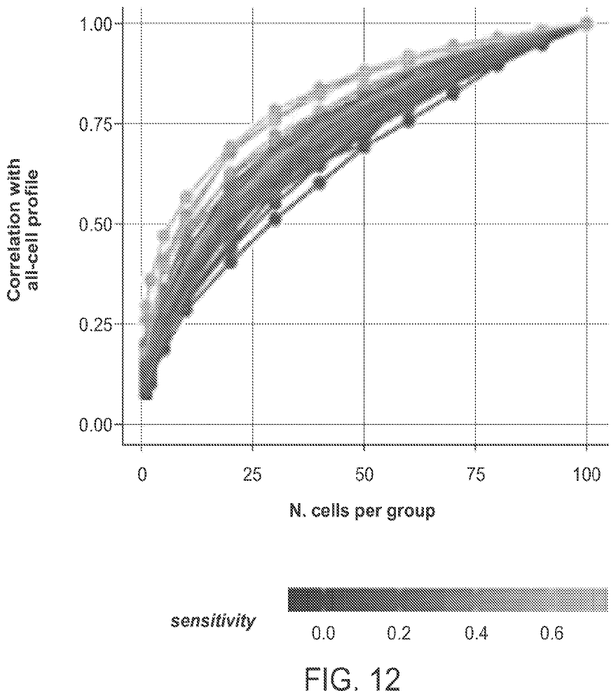

As an example, treatment of a 99 cell-line pool with the MEK inhibitor trametinib was investigated, along with the vehicle control (DMSO). More than 100 cells per cell line on average were recovered in each condition, detecting 97 of 99 cell lines with a minimum of 20 cells in each condition (average 130 cells/condition; FIGS. 2A and 2B). Down-sampling analysis suggested that measuring tens of cells per condition was sufficient to estimate each cell line's transcriptional response profile (FIG. 12). The viability-independent response to trametinib included strong down-regulation of MAPK signaling genes, including EGR1, ETV4/5, DUSP4/5/6, and SPRY2/4, KRAS signaling pathways, and TNF-alpha signaling, as well as up-regulation of the interferon response (FIG. 2C, left), consistent with previous reports (Shi-Lin et al. 2015 and Lulli et al. 2017). In contrast, the viability-related component showed strong down-regulation of cell-cycle processes (FIG. 2C, right), implicating a selective cell cycle arrest as mediating the long-term viability effects of trametinib. These results underscored how distinguishing the two response components can elucidate a drug's MoA.

Applying this analysis across all eight compounds possessing selective viability effects that were profiled with MIX-Seq, several core components of the viability-related response were found to be largely shared across compounds. These components were highly enriched for cell-cycle genes, which were selectively down-regulated in the sensitive cell lines in virtually all the selective compounds profiled (FIGS. 13A and 13B). Notably, the shared signature was also apparent in cells treated with broadly toxic compounds, such as prexasertib, the BRD2-inhibitor JQ-1, bortezomib, and gemcitabine, suggesting it reflected a general transcriptional signature of decreased cell viability and/or proliferation. The two inhibitors of anti-apoptotic proteins—navitoclax and AZD5591—were unique among the compounds tested in that they did not produce robust transcriptional response signatures, despite eliciting strong selective viability responses (particularly in the case of AZD5591).

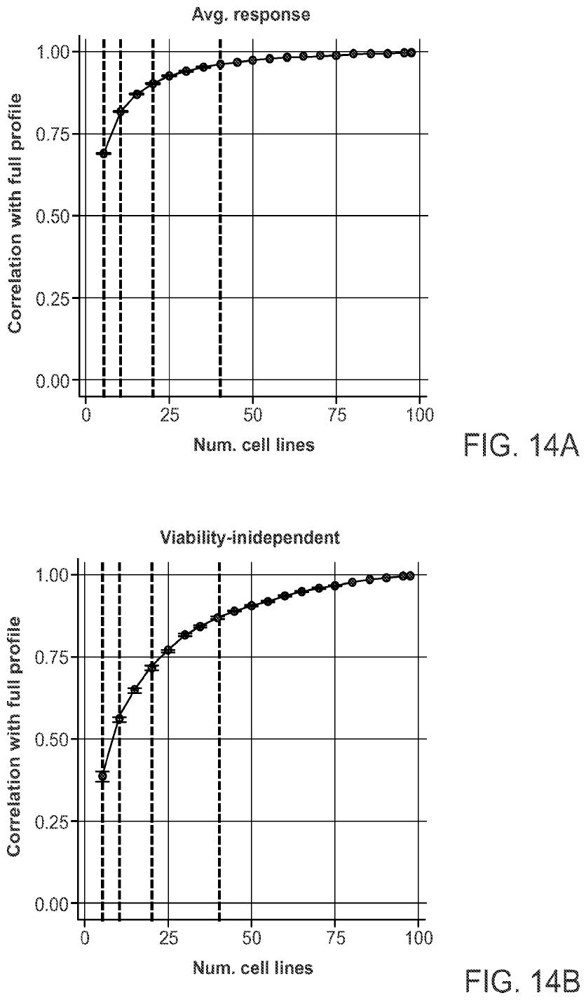

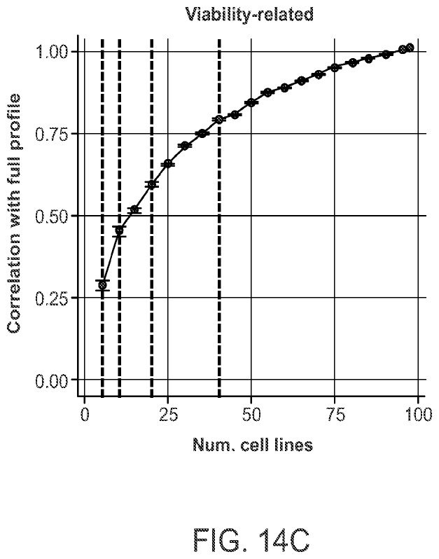

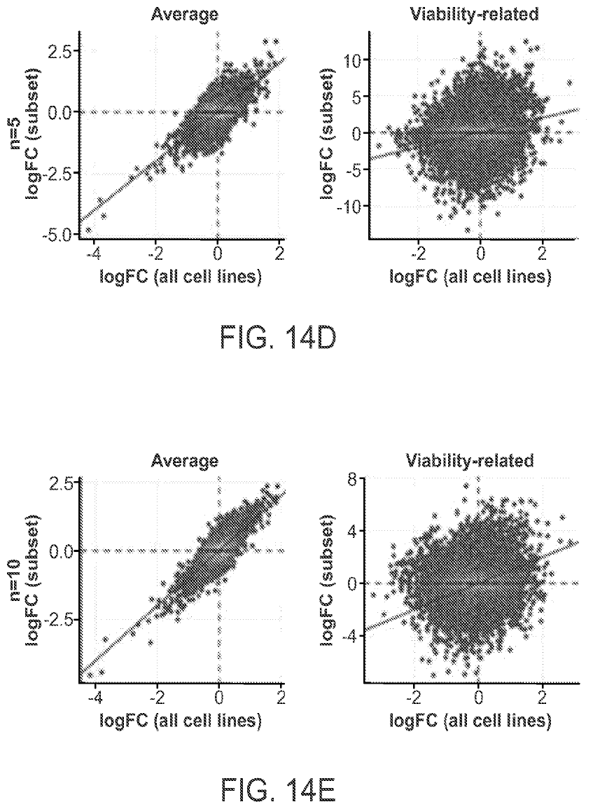

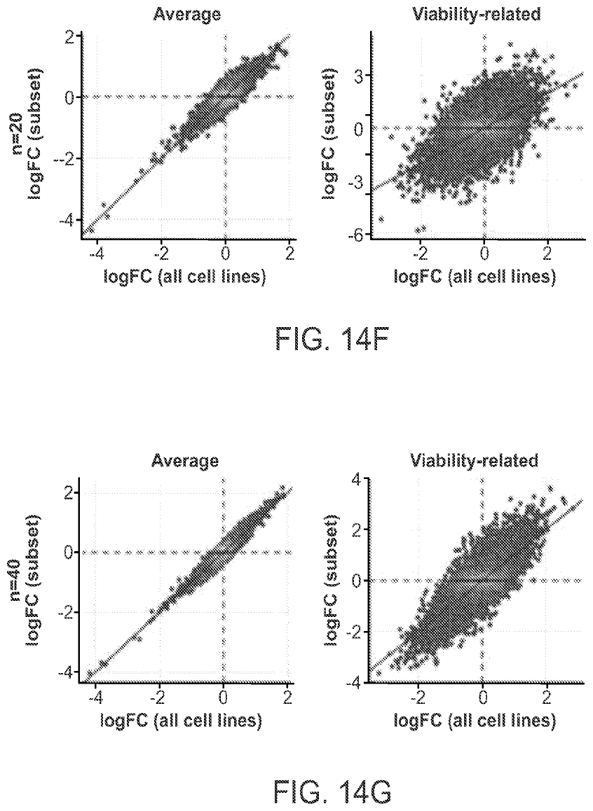

To determine how the number of different cell lines profiled impacted estimation of these transcriptional response components a down-sampling analysis was performed (FIGS. 14A-14G). While the average response across cell lines was estimated reliably from relatively few (5-10) lines, estimates of the viability-related and viability-independent response components became more robust (as measured by their similarity to estimates using all cell lines) when including data from 50 or more lines (FIGS. 14A-14G).

### Example 5: Prediction of Long-Term Viability from MIX-Seq Profiles

The ability of MIX-Seq to efficiently profile transcriptional responses across many cell lines permitted testing the feasibility of training models to predict the long-term viability effects of a drug from short-term transcriptional response measurements. Such an approach has clinical applications in therapeutic response prediction, as patient cells can be transcriptionally profiled without long term cultures. To test this, random forest models were used, assessing their accuracy using the R² of predictions on held-out test cell lines (10-fold cross-validation; see Example 1 above).

The models described herein accurately predicted across-cell line differences in viability effects for virtually all drugs tested that had selective killing profiles (with the exception of the apoptosis-inducing compounds AZD5591 and navitoclax; FIG. 3A). For several drugs, the models even predicted viability responses from transcriptional changes measured just 6 hours post-treatment. For comparison, models were also trained using the baseline ‘omics’ features of the cell lines, including their baseline expression levels (from bulk RNA-seq data) and the presence of damaging or hotspot mutations (Barretina et al. 2012 and Ghandi et al. 2019). Across drugs, transcriptional response signatures were found to be more predictive of long-term viability responses compared to the cell lines' baseline features (FIG. 3A; n=17; p=8.4×10⁻⁴; Wilcoxon signed-rank test). Notably, even when all available data were used to train models on the baseline features, such that the models had access to much larger training samples (e.g., n=741 vs. 24 cell lines for nutlin), transcriptional response profiles still compared favorably to baseline features for predicting viability effects of most drugs (FIG. 15).

A model was also successfully trained (see Example 1 above) to predict viability responses across all cell lines and drugs from transcriptional changes measured 24 hours post-treatment with good accuracy (R²=0.50), demonstrating a consistent transcriptional signature associated with viability effects across compounds. Furthermore, the genes whose transcriptional response most contributed to predicted viability effects were characterized by up-regulation of NFKB, apoptosis, and TP53 signaling, along with down-regulation of translation, cell cycle and MYC signaling (FIG. 3B), consistent with the previous analysis of viability-related response signatures (FIGS. 13A and 13B). Together, these results demonstrated that post-treatment transcriptional signatures provided a robust signal of cellular response to drugs that predicted their long-term viability effects.

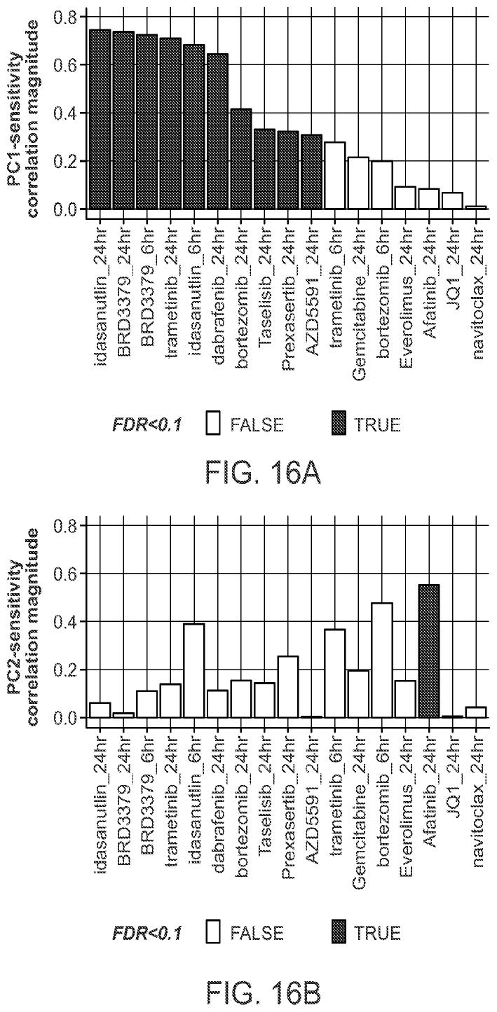

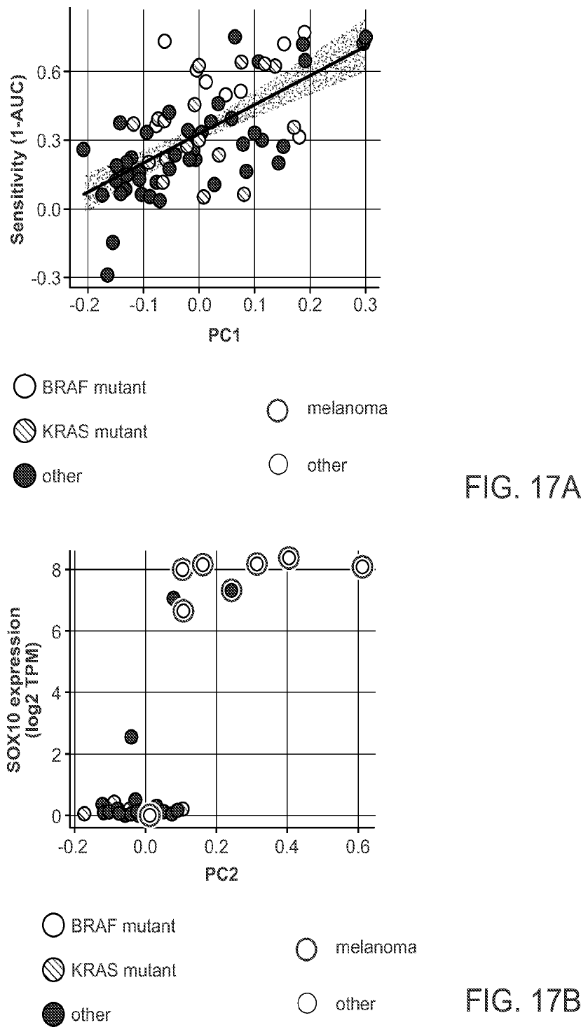

Transcriptional profiling across large panels of cell lines also enabled identification of the factors underlying their variable drug responses without a priori knowledge of the relevant genomic/molecular features driving such differences. As a simple illustration of this, principal component analysis (PCA) was applied to the matrix of trametinib responses across cell lines, measured 24 hours post-treatment (FIG. 3C). The first principal component (PCI), captured differences in trametinib sensitivity across cell lines (FIG. 3D, top and lower left). Indeed, across 9 out of thirteen tested drugs, PC1 or PC2 of the transcriptional response matrix (measured at 24 hours post-treatment) significantly correlated with the cell lines' measured drug sensitivity (FDR<0.1; FIGS. 16A and 16B), indicating this is often a predominant source of response heterogeneity. For trametinib, PC2 identified a pattern of differential response among trametinib-sensitive cell lines, distinguishing the responses of mostly BRAF mutant melanoma lines from other sensitive lines (largely KRAS mutant) (FIG. 3D, lower right). These first two PCs were also recapitulated in a separate experiment measuring trametinib responses in a different pool of cell lines (FIGS. 17A and 17B). This example highlights the power of transcriptional profiling across cell contexts to identify multiple biologically-relevant factors underlying the differential cellular response to the drug.

Finally, to identify, in an unsupervised manner, global patterns of transcriptional responses across cell lines, compounds and time points, a 2D embedding of all the combined perturbation response profiles was created with UMAP (McInnes et al. 2018). While perturbation response profiles mostly grouped by perturbation type (drug and post-treatment time point) (FIG. 3E), relationships between the set of responses for related perturbation types were also apparent. For example, responses to the same drug profiled at multiple post-treatment time points were nearby in UMAP space, and functionally related drugs such as taselisib (PIK3CAi) and everolimus (MTORi), as well as trametinib (MEKi) and afatinib (EGFRi), clustered near one another. Interestingly, the response of BRAF mutant cell lines to the BRAF inhibitor dabrafenib grouped with trametinib response profiles, rather than with the other dabrafenib responses (FIG. 3E).

### Example 6: Single-Cell Resolution Uncovered Heterogeneous Responses

In addition to the benefits of scRNA-seq as a tool for efficiently multiplexing transcriptional profiling across many cell lines, the ability to characterize responses with single-cell resolution enabled qualitatively new analyses not possible with bulk RNA-seq, and across diverse phenotypes, all assessed simultaneously with a single assay.

For example, characterizing the cell cycle effects of a perturbation yields important information about its MoA. Such measurements have heretofore typically been made using a FACS-based assay, which must be performed on each sample independently and cannot be used to relate cell cycle effects post hoc to other phenotypes without advanced planning. Using MIX-Seq, such measurements can be made in parallel across a pool of cell lines, inferring the cell cycle phase of each cell from its transcriptional profile (Tirosh et al. 2016). As a demonstration, this approach was applied to the nutlin treatment experiment (FIGS. 1A-1G), with the finding that nutlin elicited a pronounced G0/G1-arrest phenotype selectively among the TP53 WT cell lines (FIGS. 4A and 4B), as expected.

Next, the effects of each compound on the cell cycle were systematically assessed (see Example 1 above). At 24 hours post-treatment, most drugs produced an increase in the proportion of cells in G0/G1 (10/13 drugs) and concomitantly decreased the proportion of cells in S (9/13) and G2/M phases (9/13), consistent with cell cycle arrest at the G1/S transition (FIG. 4C). Two notable exceptions were the DNA-damaging agent gemcitabine and the CHEK1/2 inhibitor prexasertib. Gemcitabine also decreased the proportion of cells in G2/M but with an increase in S-phase cells, consistent with its known role in triggering CHEK1-mediated S-phase arrest. Prexasertib decreased the proportion of S-phase cells, and slightly increased the fraction of G2/M cells, consistent with inhibition of CHEK-1 mediated DNA-damage checkpoints leading to dis-regulated progression of cells through the cell cycle (King et al. 2015).

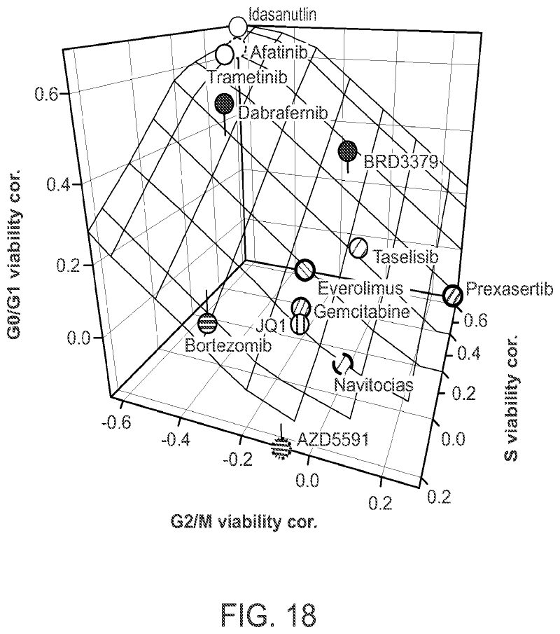

For selective compounds, cell cycle effects were also well-correlated with measured viability effects, such that drugs typically had larger effects in more sensitive cell lines (FIG. 18). The single-cell profiles were used to directly estimate drug-induced changes in relative cell abundance, with the finding that selective compounds consistently decreased the representation of more sensitive cell lines in the pool, particularly when measured 24 hours post-treatment (FIG. 19). Notably, this relationship was observed most strongly with the MCL1 inhibitor AZD5591, despite the fact that a robust transcriptional response to the drug was not observed, suggesting that direct induction of apoptosis may be detectable by selective cell line dropout in the absence of marked transcriptional changes. Together, these results demonstrated that MIX-Seq reliably read out the effects of perturbations on cell cycle progression as well as overall cell viability.

Unlike bulk expression profiling, scRNA-seq also enabled the characterization of heterogeneous responses across the cells in a population. For example, bortezomib treatment elicited a bimodal response for 10 of the 24 cell lines in the pool (FIGS. 4D and 4E). All 10 cases showed a similar pattern, with one cell subset arresting in G0/G1 and another composed of predominantly S-phase cells (FIGS. 4E and 4F). In contrast, more homogenous population responses were observed for the other drugs tested.

When cell lines are composed of transcriptionally distinct subsets even in the absence of perturbations, as shown by Kinker et al. 2019, MIX-Seq examines whether different cell populations within a given sample exhibit differential treatment responses. For example, the lung cell line IALM had two distinct subpopulations at baseline, characterized by differential expression of EMT and integrin-related programs (FIGS. 20A and 20B). Indeed, these subpopulations exhibited subtle but significant differences in their response to trametinib treatment (FIGS. 20A and 20B). These examples highlighted the ability of MIX-Seq to reveal heterogeneous responses that would be missed by bulk transcriptional profiling.

### Example 7: Multiplexed Profiling Across Post-Treatment Time Points

Many perturbations elicit cellular responses that evolve over time, suggesting that more information could be obtained by profiling cells across a sequence of post-treatment time points. Several methods were developed for introducing sample-specific barcodes to allow multiplexing of scRNA-seq measurements across experimental conditions and time points (Stoeckius et al. 2018 and Shin et al. 2019). In particular, Cell Hashing (Stoeckius et al. 2018) uses oligonucleotide-conjugated antibodies against cell-surface antigens (called hashtags) to label cells with unique barcodes for each experimental condition. Since MIX-Seq uses naturally occurring SNP barcodes to multiplex cell lines, it is easily combined with such approaches to allow for dual-multiplexing of cell lines and experimental conditions with a single scRNA-seq readout.

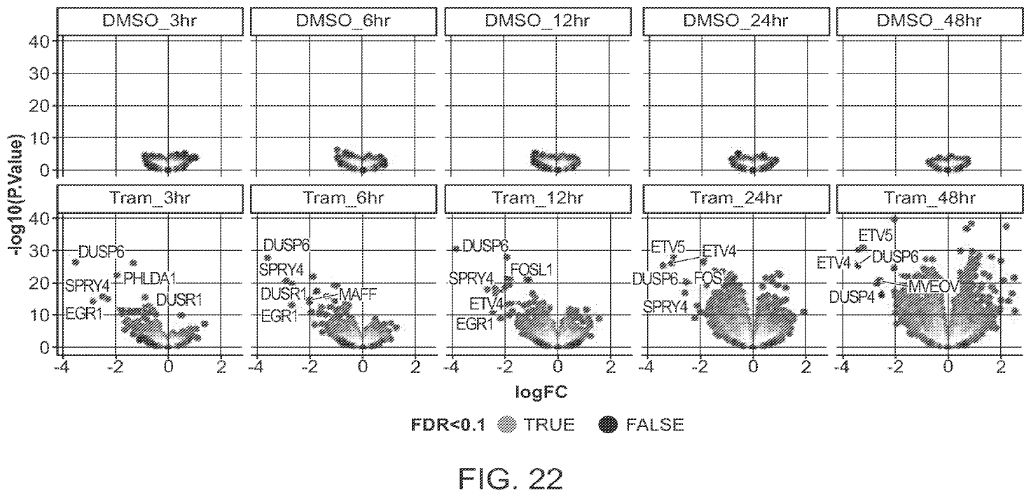

Leveraging this, responses of a pool of 24 cell lines to trametinib were measured along five time points, ranging from 3 to 48 hours post-treatment, using Cell Hashing to multiplex treatment conditions (FIG. 5A). As controls, DMSO-treated samples were included at each of the 5 time points, in addition to untreated samples, for a total of 11 conditions. Hashtag reads provided robust labeling of treatment conditions, with good tagging efficiency across all cell lines (FIGS. 21A and 21B). Since substantial differences in DMSO-treated cells were not observed across time points (FIG. 22), they were pooled together for subsequent analysis, yielding a total of 13,713 clearly-tagged single cells across all treatment conditions and cell lines.

The single-cell expression profiles illustrated strong time-dependent changes in response to trametinib, whose magnitude varied considerably across cell lines (FIG. 5B). To better understand these changes, the temporal transcriptional changes of key trametinib-response genes were examined. For example, EGR1, an immediate early response gene known to be activated by MAPK signaling (Lim et al. 1998), was dramatically down-regulated 3 hours after trametinib treatment in both the sensitive cell line RCM1 and the insensitive line TEN (FIG. 5C). In contrast, MCMI, a cell-cycle-related gene that was part of the viability-related response, was selectively down-regulated only in the sensitive line RCM1, and only after 12-24 hours post-treatment (FIG. 5D).

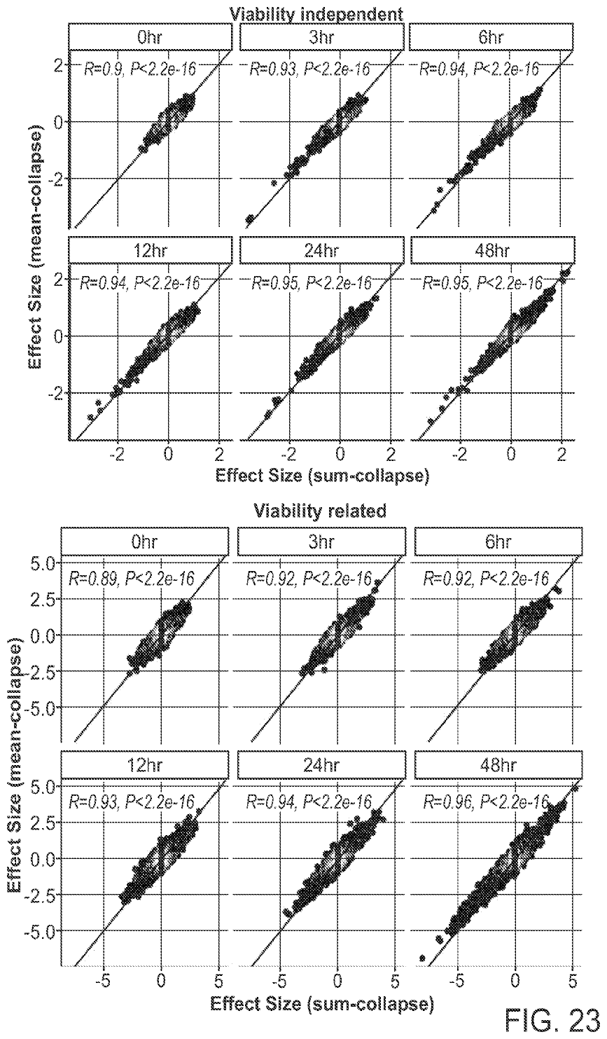

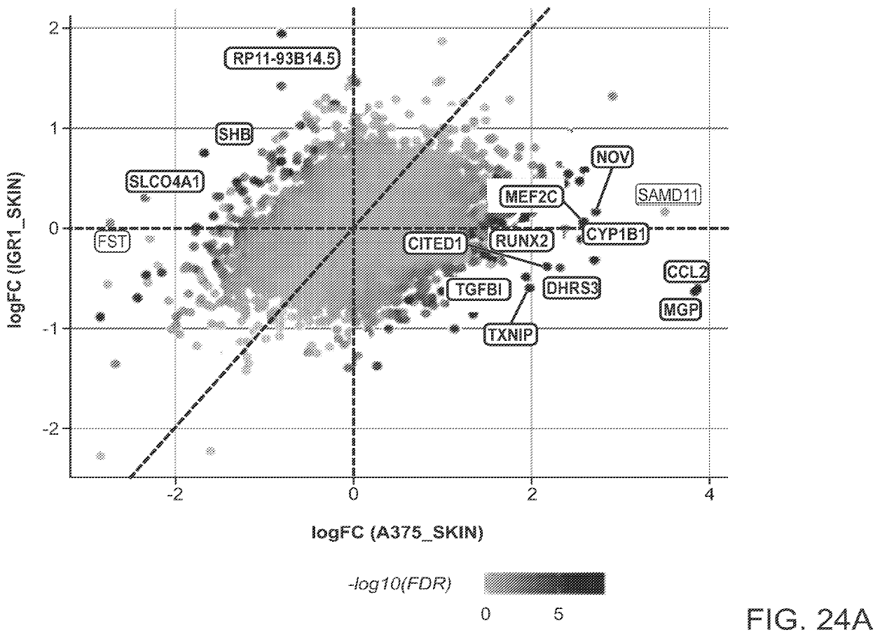

The statistical model developed herein was next applied (FIG. 2C) to quantify the temporal evolution of viability-related and viability-independent components of the trametinib response for each gene, integrating across all cell lines. Down-regulated genes in the viability-independent response showed a range of temporal patterns, with several (such as EGR1 and DUSP6) reaching maximal down-regulation 3 hours post-treatment (FIG. 5E). In contrast, the viability-related response emerged much later, with genes such as GINS2, E2F1, and MCM4 showing selective down-regulation in sensitive cell lines only 12-24 hours post-treatment (FIG. 5F). These results were confirmed to not be biased by temporal variation in the numbers of cells available to estimate each cell line's response (FIG. 23). These differences were also characterized at the pathway level, with the finding that the viability-independent down-regulation of the KRAS signaling pathway emerged 3 hours after treatment (FIG. 5E), while the viability-related down-regulation of cell cycle genes started 24 hours after treatment (FIG. 5F). The latter was also consistent with the time course of G0/G1-arrest based on inferred cell cycle phases (FIG. 5G).

These results thus highlighted the utility of large-scale transcriptional profiling, both across cell lines and time points, to identify the different components of drug response. The ability to separate these transcriptional components provides an understanding of the initial effects of target-engagement, the mechanism underlying selective loss of cell viability, as well as compound MoA.

### Example 8: Therapeutic Response Evaluation Through Assessment of Transcriptome Sequencing (TREAT-Seq)—Treatment Response Prediction to Standard and Experimental Treatment Regimens

As described above for the instant MIX-seq platform, strong correlations of post-treatment RNA signatures with treatment responses have now been identified. The instant example describes the development herein of Therapeutic Response Evaluation through Assessment of Transcriptome Sequencing (TREAT-Seq), disclosed as an approach to generate and analyze post-treatment RNA profiles capable of predicting treatment responsiveness, optionally within as little as 1 week (or less) after biopsy (via comparison of such profiles with established drug-responsive transcriptional signatures). Such an accelerated time frame (relative to response prediction methods known in the art, e.g., those based upon assessment of organoids) allows for implementation of TREAT-Seq functional profiling to guide choice of therapy in clinical care settings. A key concept of TREAT-Seq is that gene expression profiles derived from cancer cells after short-term treatment with therapeutic compounds can accurately predict longer term responses of these models to those therapies in conventional cell viability assays. Post-treatment gene expression profiles derived from biopsy cells treated ex vivo can therefore be used to predict patient responses to therapeutic regimens, thereby providing a path forward for functional precision medicine.

As an exemplary implementation of TREAT-seq, a needle biopsy is obtained in the course of clinical care, and this core needle biopsy is dissociated and treated ex vivo with clinically available compounds. Single-cell RNA sequencing is employed to measure post-treatment gene expression features at 12-48 hours post-biopsy on the small number of cells available from this needle biopsy (˜25-50,000 cells per biopsy). Based on the induction of treatment response profiles, therapeutic efficacy predictions are made to inform clinical care. TREAT-Seq addresses several needs/advantages of an early predictive assay, including the ability to profile treatment response upon smaller numbers of cells, even in the presence of other cell types from the tumor microenvironment, with available sequencing-based technology.

TREAT-Seq is used, for example, to evaluate the impact of conventional cancer treatment regimens on the cancer cell transcriptional programs of a biopsy test sample, to design relevant patient-specific combination therapy strategies based on the alterations in cell state evoked by those treatment regimens. Thus, a biopsy is obtained and dissociated cells are treated ex vivo with relevant compounds. Gene expression programs are assayed using single-cell RNA sequencing to evaluate small numbers of cells (available from a needle biopsy), to interrogate compensatory resistance mechanisms. Personalized combination treatment regimens are then designed to combat those resistance mechanisms.

To predict therapeutic responsiveness, TREAT-Seq identifies post-treatment gene expression profiles, examined within hours of biopsy after ex vivo sample treatment. In a first phase of analysis, patient-derived models are used to examine the correlation of novel TREAT-Seq profiles with drug sensitivity from a standard 5-day Cell-Titer-Glo (CTG) assay for a range of drugs, including conventional chemotherapy agents and targeted therapies such as MEK or ERK inhibitors. An established biobank of 100 clinically annotated human pancreatic cancer organoid models for which genomic and CTG drug sensitivity profiling has already been performed is employed, for purpose of comparison (e.g., to build reference test drug-responsive transcriptional signatures). In a second phase, the power of novel TREAT-Seq profiles to predict drug response is prospectively established by leveraging clinical trials, e.g., in pancreatic cancer, including a Phase II study of ERK1/2 inhibition alone or in combination with the autophagy inhibitor hydroxychloroquine in pancreatic cancer patients; and a Phase II study of gemcitabine/nab-paclitaxel in combination with ERK1/2 inhibition in advanced pancreatic cancer patients. For each patient in these trials, TREAT-Seq is performed using relevant drug combinations on biopsy specimens. In parallel, organoid models are derived and conventional drug sensitivity testing is performed for comparative analysis with the instant TREAT-Seq approach. These studies are expected to further support that TREAT-Seq profiles obtained from ex vivo treatment of biopsy specimens (and reference TREAT-Seq transcriptional signatures) can serve as novel functional biomarkers of drug response. TREAT-Seq is also evaluated and implemented for selection of first-line chemotherapy regimens in other cancer types.

### Data Availability

All data reported in the instant disclosure, including single-cell RNA-sequencing data, drug sensitivity measures, and other cell line features used in the analysis, are available on figshare at www.figshare.com/s/139f64b495dea9d88c70, (Cancer Data Science 2019). Additional data used in the analysis are also publicly available. Baseline cell line omics features and CRISPR genetic dependency data are taken from the 19Q3 DepMap dataset (Broad DepMap 2019); available at www.depmap.org or from figshare at www.figshare.com/articles/DepMap_19Q3_Public/9201770/2. The cell line drug sensitivity data were taken from the Sanger GDSC dataset (Iorio et al. 2016 and Garnett et al. 2012), which is available for download from www.depmap.org or www.cancerrxgene.org/, and data generated using the PRISM multiplexed drug screening platform (Corsello et al. 2020 and Yu et al. 2016), which is available for download from www.depmap.org. The L1000 gene expression signatures were taken from either the LINCS Phase 2 data (GEO accession GSE70138, downloaded from www.amp.pharm.mssm.edu/Slicr) or LINCS Phase 1 data (GEO accession GSE92742, downloaded from www.clue.io).

### Code Availability

Custom code used in the analysis, and for generating all figures, is available at www.github.com/broadinstitute/mix_seq_ms. Code used for SNP classification is available at www.github.com/broadinstitute/single_cell_classification.

### REFERENCES

Adamson, B., Norman, T. M., Jost, M., et al. 2016. A Multiplexed Single-Cell CRISPR Screening Platform Enables Systematic Dissection of the Unfolded Protein Response. Cell 167(7), pp. 1867-1882.e21.
Barretina, J., Caponigro, G., Stransky, N., et al. 2012. The Cancer Cell Line Encyclopedia enables predictive modelling of anticancer drug sensitivity. Nature 483(7391), pp. 603-607.
Basu, A. et al. 2013. An interactive resource to identify cancer genetic and lineage dependencies targeted by small molecules. Cell 154, 1151-1161.
Behan, F. M., Iorio, F., Picco, G., et al. 2019. Prioritization of cancer therapeutic targets using CRISPR-Cas9 screens. Nature 568(7753), pp. 511-516.
Ben-David, U., Siranosian, B., Ha, G., et al. 2018. Genetic and transcriptional evolution alters cancer cell line drug response. Nature 560(7718), pp. 325-330.
Benjamini, Y. & Hochberg, Y. 1995. Controlling the false discovery rate: A practical and powerful approach to multiple testing. Journal of the Royal Statistical Society: Series B (Methodological) 57, 289-300.
Broad DepMap. DepMap 19Q3 Public. 2019. Figshare. doi:10.6084/m9.figshare.9201770.v3
Bush, E. C., Ray, F., Alvarez, M. J., et al. 2017. PLATE-Seq for genome-wide regulatory network analysis of high-throughput screens. Nature Communications 8(1), p. 105.
Butler, A., Hoffman, P., Smibert, P., Papalexi, E. and Satija, R. 2018. Integrating single-cell transcriptomic data across different conditions, technologies, and species. Nature Biotechnology 36(5), pp. 411-420.
Calle, Y., Palomares, T., Castro, B., del Olmo, M. and Alonso-Varona, A. 2000. Removal of N-glycans from cell surface proteins induces apoptosis by reducing intracellular glutathione levels in the rhabdomyosarcoma cell line S4MH. Biology of the Cell 92(8-9), pp. 639-646.
Cancer Data Science. 2019. MIX-seq data. figshare. Dataset. www://doi.org/10.6084/m9.figshare.10298696.v1
Charafe-Jauffret, E., Ginestier, C., lovino, F., et al. 2009. Breast cancer cell lines contain functional cancer stem cells with metastatic capacity and a distinct molecular signature. Cancer Research 69(4), pp. 1302-1313.
Crowell, H. L. et al. 2019. On the discovery of population-specific state transitions from multi-sample multi-condition single-cell RNA sequencing data. Preprint at www.biorxiv.org/content/10.1101/713412v1.
Corsello, S. M. et al. 2020. Discovering the anticancer potential of non-oncology drugs by systematic viability profiling. Nat. Cancer. doi:10.1038/s43018-019-0018-6.
Dixit, A., Parnas, O., Li, B., et al. 2016. Perturb-Seq: Dissecting Molecular Circuits with Scalable Single-Cell RNA Profiling of Pooled Genetic Screens. Cell 167(7), pp. 1853-1866.e17.
Friedman, J., Hastie, T. and Tibshirani, R. 2010. Regularization Paths for Generalized Linear Models via Coordinate Descent. Journal of statistical software 33(1), pp. 1-22.
Garnett, M. J., Edelman, E. J., Heidorn, S. J., et al. 2012. Systematic identification of genomic markers of drug sensitivity in cancer cells. Nature 483(7391), pp. 570-575.
Garrison, E. and Marth, G. 2012. Haplotype-based variant detection from short-read sequencing. arXiv.
Gaublomme, J. T., Li, B., McCabe, C., et al. 2019. Nuclei multiplexing with barcoded antibodies for single-nucleus genomics. Nature Communications 10(1), p. 2907.
Ghandi, M. et al. 2019. Next-generation characterization of the Cancer Cell Line Encyclopedia. Nature 569, 503-508.
Hafemeister, C. and Satija, R. 2019. Normalization and variance stabilization of single-cell RNA-seq data using regularized negative binomial regression. BioRxiv.
Han, S., Ren, Y., He, W., et al. 2018. ERK-mediated phosphorylation regulates SOX10 sumoylation and targets expression in mutant BRAF melanoma. Nature Communications 9(1), p. 28.
Huang, Y., McCarthy, D. J. & Stegle, O. Vireo: 2019. Bayesian demultiplexing of pooled single-cell RNA-seq data without genotype reference. Preprint at www.biorxiv.org/content/10.1101/598748v1. doi:10.1101/598748
Iorio, F., Knijnenburg, T. A., Vis, D. J., et al. 2016. A landscape of pharmacogenomic interactions in cancer. Cell 166(3), pp. 740-754.
Janitza, S., Celik, E. and Boulesteix, A.-L. 2016. A computationally fast variable importance test for random forests for high-dimensional data. Advances in data analysis and classification.
Jeganathan, S. and Lee, J. M. 2007. Binding of elongation factor eEF1A2 to phosphatidylinositol 4-kinase beta stimulates lipid kinase activity and phosphatidylinositol 4-phosphate generation. The Journal of Biological Chemistry 282(1), pp. 372-380.
Jones, A., Tsherniak, A. & McFarland, J. 2020. Post-perturbational transcriptional signatures of cancer cell line vulnerabilities. Preprint at www.biorxiv.org/content/10.1101/2020.03.04.976217v1. doi:10.1101/2020.03.04.976217
Kang, H. M., Subramaniam, M., Targ, S., et al. 2018. Multiplexed droplet single-cell RNA-sequencing using natural genetic variation. Nature Biotechnology 36(1), pp. 89-94.
King, C. et al. LY2606368 Causes Replication Catastrophe and Antitumor Effects through CHK1-Dependent Mechanisms. Mol. Cancer Ther. 14, 2004-2013 (2015).
Kinker, G. S. et al. 2019. Pan-cancer single cell RNA-seq uncovers recurring programs of cellular heterogeneity. Preprint at www.biorxiv.org/content/10.1101/807552v1. doi:10.1101/807552
Klein, A. M. et al. 2015. Droplet barcoding for single-cell transcriptomics applied to embryonic stem cells. Cell 161, 1187-1201.
Kodack, D. P. et al. 2017. Primary Patient-Derived Cancer Cells and Their Potential for Personalized Cancer Patient Care. Cell Rep. 21, 3298-3309.
Lamb, J. et al. 2003. A mechanism of cyclin D1 action encoded in the patterns of gene expression in human cancer. Cell 114, 323-334.
Lamb, J., Crawford, E. D., Peck, D., et al. 2006. The Connectivity Map: using gene-expression signatures to connect small molecules, genes, and disease. Science 313(5795), pp. 1929-1935.
Law, C. W., Chen, Y., Shi, W. and Smyth, G. K. 2014. voom: Precision weights unlock linear model analysis tools for RNA-seq read counts. Genome Biology 15(2), p. R29.
Li, B. et al. 2019. Cumulus: a cloud-based data analysis framework for large-scale single-cell and single-nucleus RNA-seq. Preprint at www.biorxiv.org/content/10.1101/823682v1. doi: 10.1101/823682
Liberzon, A., Subramanian, A., Pinchback, R., Thorvaldsdóttir, H., Tamayo, P. and Mesirov, J. P. 2011. Molecular signatures database (MSigDB) 3.0. Bioinformatics 27(12), pp. 1739-1740.
Lim, C. P., Jain, N. & Cao, X. 1998. Stress-induced immediate-early gene, egr-1, involves activation of p38/JNK1. Oncogene 16, 2915-2926.
Lulli, D., Carbone, M. L. and Pastore, S. 2017. The MEK inhibitors trametinib and cobimetinib induce a type I interferon response in human keratinocytes. International Journal of Molecular Sciences 18(10).
Lun, A. T. L., Chen, Y. and Smyth, G. K. 2016. It's DE-licious: A Recipe for Differential Expression Analyses of RNA-seq Experiments Using Quasi-Likelihood Methods in edgeR. Methods in Molecular Biology 1418, pp. 391-416.
Lun, A. T. L. and Marioni, J. C. 2017. Overcoming confounding plate effects in differential expression analyses of single-cell RNA-seq data. Biostatistics 18(3), pp. 451-464.
Macosko, E. Z., Basu, A., Satija, R., et al. 2015. Highly Parallel Genome-wide Expression Profiling of Individual Cells Using Nanoliter Droplets. Cell 161(5), pp. 1202-1214.
McDonald, E. R., de Weck, A., Schlabach, M. R., et al. 2017. Project DRIVE: A Compendium of Cancer Dependencies and Synthetic Lethal Relationships Uncovered by Large-Scale, Deep RNAi Screening. Cell 170(3), pp. 577-592.e10.
McInnes, L., Healy, J. & Melville, J. 2018. UMAP: Uniform Manifold Approximation and Projection for Dimension Reduction. Preprint at www.arxiv.org/abs/1802.03426.
Meyers, R. M., Bryan, J. G., McFarland, J. M., et al. 2017. Computational correction of copy number effect improves specificity of CRISPR-Cas9 essentiality screens in cancer cells. Nature Genetics 49(12), pp. 1779-1784.
Nagai, Y., Miyazawa, H., Huqun, et al. 2005. Genetic heterogeneity of the epidermal growth factor receptor in non-small cell lung cancer cell lines revealed by a rapid and sensitive detection system, the peptide nucleic acid-locked nucleic acid PCR clamp. Cancer Research 65(16), pp. 7276-7282.
Norman, T. M., Horlbeck, M. A., Replogle, J. M., et al. 2019. Exploring genetic interaction manifolds constructed from rich phenotypes. BioRxiv.
Ritchie, M. E., Phipson, B., Wu, D., et al. 2015. limma powers differential expression analyses for RNA-sequencing and microarray studies. Nucleic Acids Research 43(7), p. e47.
Robinson, M. D., McCarthy, D. J. and Smyth, G. K. 2010. edgeR: a Bioconductor package for differential expression analysis of digital gene expression data. Bioinformatics 26(1), pp. 139-140.
Roschke, A. V., Tonon, G., Gehlhaus, K. S., et al. 2003. Karyotypic complexity of the NCI-60 drug-screening panel. Cancer Research 63(24), pp. 8634-8647.
Scrucca, L., Fop, M., Murphy, T. B. & Raftery, A. E. 2016. mclust 5: Clustering, Classification and Density Estimation Using Gaussian Finite Mixture Models. R J. 8, 289-317.
Shaffer, S. M., Dunagin, M. C., Torborg, S. R., et al. 2017. Rare cell variability and drug-induced reprogramming as a mode of cancer drug resistance. Nature 546(7658), pp. 431-435.
Shi-Lin, D., Yuan, X., Zhan, S., Luo-Jia, T. and Chao-Yang, T. 2015. Trametinib, a novel MEK kinase inhibitor, suppresses lipopolysaccharide-induced tumor necrosis factor (TNF)-α production and endotoxin shock. Biochemical and Biophysical Research Communications 458(3), pp. 667-673.
Shin, D., Lee, W., Lee, J. H. and Bang, D. 2019. Multiplexed single-cell RNA-seq via transient barcoding for simultaneous expression profiling of various drug perturbations. Science Advances 5(5), p. eaav2249.
Sivan, G. and Elroy-Stein, O. 2008. Regulation of mRNA Translation during cellular division. Cell Cycle 7(6), pp. 741-744.
Soneson, C. and Robinson, M. D. 2018. Bias, robustness and scalability in single-cell differential expression analysis. Nature Methods.
Srivatsan, S. R. et al. 2019. Massively multiplex chemical transcriptomics at single cell resolution. Science.
Stoeckius, M., Zheng, S., Houck-Loomis, B., et al. 2018. Cell Hashing with barcoded antibodies enables multiplexing and doublet detection for single cell genomics. Genome Biology 19(1), p. 224.
Subramanian, A., Narayan, R., Corsello, S. M., et al. 2017. A next generation connectivity map: L1000 platform and the first 1,000,000 profiles. Cell 171(6), pp. 1437-1452.e17.
Szalai, B. et al. 2019. Signatures of cell death and proliferation in perturbation transcriptomics data-from confounding factor to effective prediction. Nucleic Acids Res. 47, 10010-10026.
Tirosh, I., Izar, B., Prakadan, S. M., et al. 2016. Dissecting the multicellular ecosystem of metastatic melanoma by single-cell RNA-seq. Science 352(6282), pp. 189-196.
Tseng, Y.-Y. & Boehm, J. S. 2019. From cell lines to living biosensors: new opportunities to prioritize cancer dependencies using ex vivo tumor cultures. Curr. Opin. Genet. Dev. 54, 33-40.
Tsherniak, A., Vazquez, F., Montgomery, P. G., et al. 2017. Defining a cancer dependency map. Cell 170(3), pp. 564-576.e16.
Vassilev, L. T. et al. 2004. In vivo activation of the p53 pathway by small-molecule antagonists of MDM2. Science 303, 844-848.
Xu, J. et al. 2019. Genotype-free demultiplexing of pooled single-cell RNA-seq. Genome Biol. 20, 290.
Ye, C., Ho, D. J., Neri, M., et al. 2018. DRUG-seq for miniaturized high-throughput transcriptome profiling in drug discovery. Nature Communications 9(1), p. 4307.
Yuan, H., Yan, M., Zhang, G., et al. 2018. CancerSEA: a cancer single-cell state atlas. Nucleic Acids Research 47.
Yu, C., Mannan, A. M., Yvone, G. M., et al. 2016. High-throughput identification of genotype-specific cancer vulnerabilities in mixtures of barcoded tumor cell lines. Nature Biotechnology 34(4), pp. 419-423.
Zheng, G. X. Y., Terry, J. M., Belgrader, P., et al. 2017. Massively parallel digital transcriptional profiling of single cells. Nature Communications 8, p. 14049.

All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

One skilled in the art would readily appreciate that the present disclosure is well adapted to carry out the objects and obtain the ends and advantages mentioned, as well as those inherent therein. The methods and compositions described herein as presently representative of preferred embodiments are exemplary and are not intended as limitations on the scope of the disclosure. Changes therein and other uses will occur to those skilled in the art, which are encompassed within the spirit of the disclosure, are defined by the scope of the claims.

In addition, where features or aspects of the disclosure are described in terms of Markush groups or other grouping of alternatives, those skilled in the art will recognize that the disclosure is also thereby described in terms of any individual member or subgroup of members of the Markush group or other group.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Embodiments of this disclosure are described herein, including the best mode known to the inventors for carrying out the disclosed invention. Variations of those embodiments may become apparent to those of ordinary skill in the art upon reading the foregoing description.

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present disclosure provides preferred embodiments, optional features, modification and variation of the concepts herein disclosed may be resorted to by those skilled in the art, and that such modifications and variations are considered to be within the scope of this disclosure as defined by the description and the appended claims.

It will be readily apparent to one skilled in the art that varying substitutions and modifications can be made to the invention disclosed herein without departing from the scope and spirit of the invention. Thus, such additional embodiments are within the scope of the present disclosure and the following claims. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context. Those skilled in the art will recognize, or be able to ascertain using no more than routine experimentation, many equivalents to the specific embodiments of the disclosure described herein. Such equivalents are intended to be encompassed by the following claims.

1. A method for selecting a drug for treatment of a subject having or at risk of developing a disease or disorder, the method comprising:
a) obtaining a population of cells from the subject;
b) contacting the population of cells with a test drug;
c) obtaining one or more single cell transcriptional profiles of the population of cells from the subject;
d) comparing the one or more single cell transcriptional profiles of the population of cells from the subject with a reference test drug-responsive transcriptional signature;
e) identifying a match between the one or more single cell transcriptional profiles of the population of cells from the subject and the reference test drug-responsive transcriptional signature; and
f) selecting the test drug for administration to the subject,
thereby selecting a drug for treatment of a subject having or at risk of developing a disease or disorder.

2. The method of claim 1, wherein the one or more single cell transcriptional profiles are obtained within a week of obtaining the population of cells from the subject, optionally within 48 hours of obtaining the population of cells from the subject, optionally within 24 hours of obtaining the population of cells from the subject, optionally in 12-24 hours of obtaining the population of cells from the subject.

3. The method of claim 1, wherein the one or more single cell transcriptional profiles are obtained at multiple timepoints, optionally at two or more timepoints between 3 and 48 hours after contacting the population of cells with the test drug in step (b).

4. The method of claim 1, wherein the population of cells of step (a) is obtained from a needle biopsy and/or a tumor biopsy, optionally wherein the biopsy comprises a heterogeneous population of cells, optionally wherein the biopsy comprises between about 25,000 to about 50,000 cells.

5. The method of claim 1, wherein the test drug is selected from the group consisting of a small molecule, a nucleic acid and a peptide.

6. The method of claim 1, wherein the test drug is selected from the group consisting of a cytotoxic chemotherapy, a targeted signaling pathway inhibitor (e.g., an EGFR inhibitor and/or a KRAS inhibitor), an anti-hormonal therapy (e.g., an anti-androgen and/or an anti-estrogen), a DNA damage repair inhibitor and an epigenetic inhibitor (e.g., a Dnmt2 inhibitor).

7. The method of claim 1, wherein the test drug is selected from the group consisting of dabrafenib, trametinib, bortezomib, nutlin, navitoclax, everolimus, CGS15943, AZD5591, afatinib, JQ1, gemcitabine, taselisib and prexasertib.

8. The method of claim 1, wherein the subject has a neoplasia, optionally wherein the neoplasia is a cancer selected from the group consisting of carcinoma or sarcoma of the head, neck, lung, esophagus, stomach, small intestine, pancreas, gall bladder, biliary ducts, liver, kidney, adrenal gland, colon, rectum, anus, skin, connective tissues, blood vessels, muscle, bone and brain.

9. The method of claim 1, wherein the subject has a cancer selected from the group consisting of a cancer of the blood, a cancer of the bone marrow, a cancer of the lymph nodes, a cancer of the spleen and a cancer of the immune system.

10. The method of claim 1, wherein the subject has a non-cancerous neoplastic condition, optionally wherein the non-cancerous neoplastic condition is selected from the group consisting of a hyperproliferative blood cell condition (e.g., a myeloproliferative neoplasm, an eosinophilic syndrome, Sweet's syndrome, Hemophagocytic lymphohistiocytosis (HLH) and related conditions).

11. The method of claim 1, wherein the subject has a microbial disease or disorder, optionally wherein the microbial disease or disorder is a microbial condition for which therapy selection and/or administration comprises ex vivo therapy to bacterial or vival cultures for evaluation of antibiotic susceptibility.

12. The method of claim 1, wherein the reference test drug-responsive transcriptional signature is obtained from transcriptome sequencing of known test drug-responsive cell lines or test drug-responsive organoids.

13. The method of claim 1, wherein the reference test drug-responsive transcriptional signature is obtained from transcriptome profiling of known test drug-responsive cell lines at multiple timepoints after administration of test drug.

14. The method of claim 1, wherein the reference test drug-responsive transcriptional signature is obtained or refined using machine learning.

15. The method of claim 1, wherein identifying a match between the one or more single cell transcriptional profiles of the population of cells from the subject and the reference test drug-responsive transcriptional signature in step (e) comprises comparing one or more principal components of the reference test drug-responsive transcriptional signature with the one or more single cell transcriptional profiles of the population of cells from the subject and identifying the one or more principal components of the reference test drug-responsive transcriptional signature in the one or more single cell transcriptional profiles of the population of cells from the subject.

16. The method of claim 15, wherein a single principal component is identified as a match between the reference test drug-responsive transcriptional signature and the one or more single cell transcriptional profiles of the population of cells from the subject.

17. The method of claim 15, wherein two or more principal components are identified as a match between the reference test drug-responsive transcriptional signature and the one or more single cell transcriptional profiles of the population of cells from the subject, optionally wherein three or more principal components are identified as a match between the reference test drug-responsive transcriptional signature and the one or more single cell transcriptional profiles of the population of cells from the subject, optionally wherein four or more principal components are identified as a match between the reference test drug-responsive transcriptional signature and the one or more single cell transcriptional profiles of the population of cells from the subject;
a selection of principal components of the reference test drug-responsive transcriptional signature is identified in the one or more single cell transcriptional profiles of the population of cells from the subject; and/or
all identified principal components of the reference test drug-responsive transcriptional signature are also identified in the one or more single cell transcriptional profiles of the population of cells from the subject.

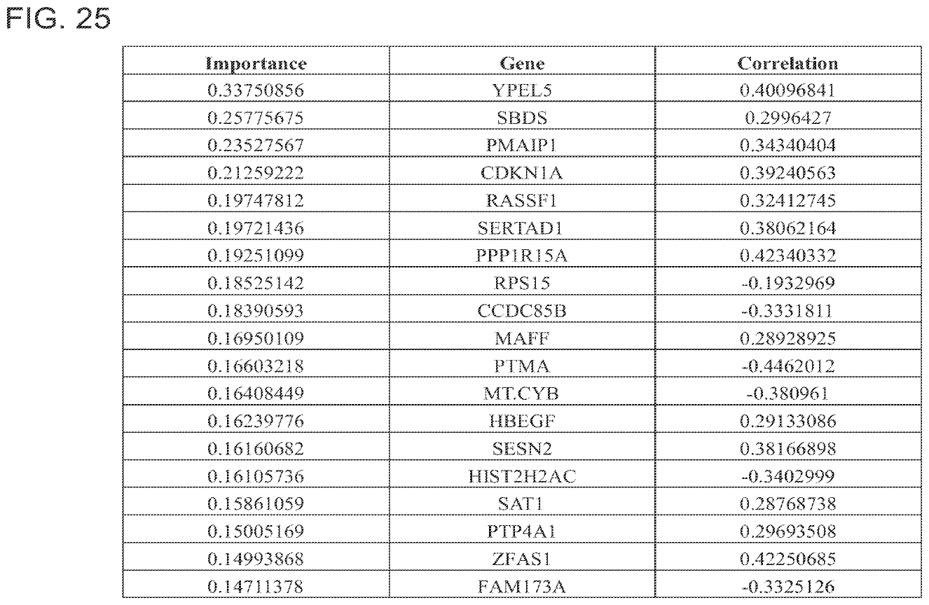

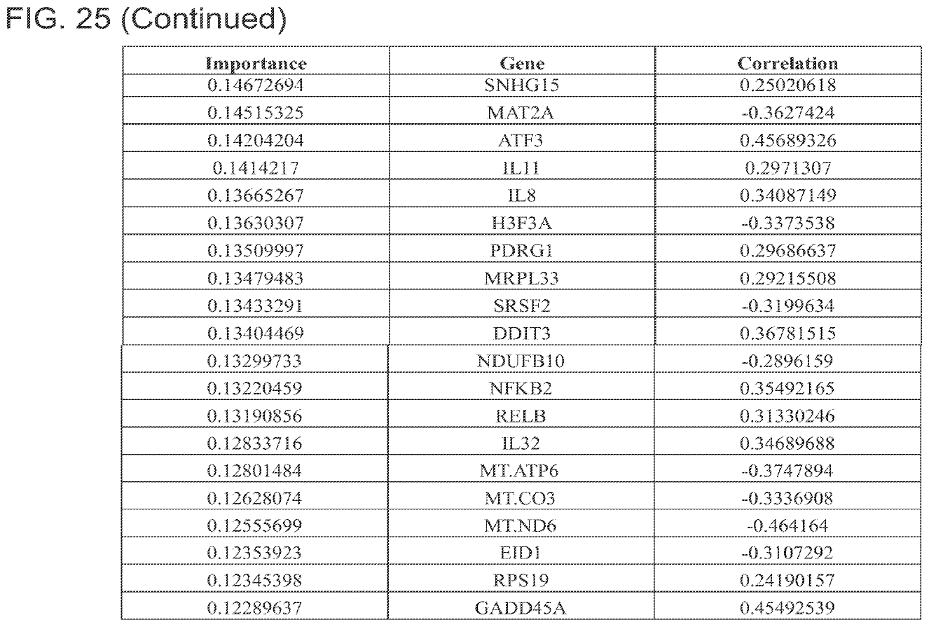

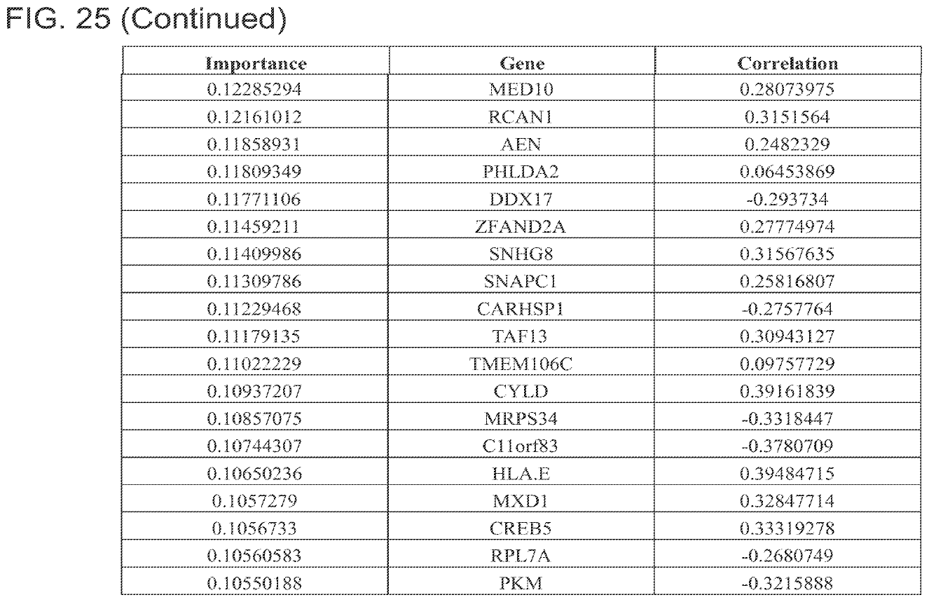

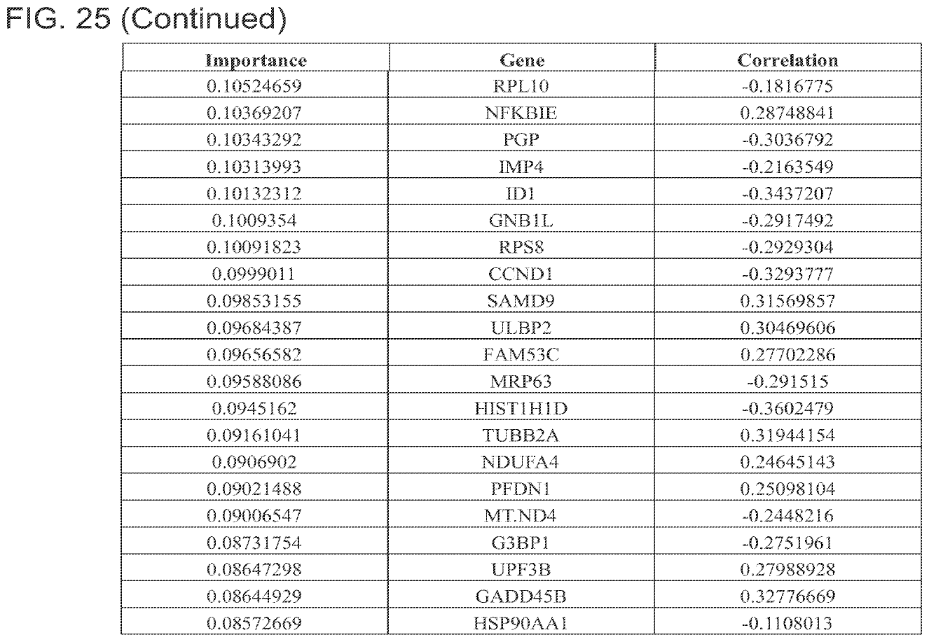

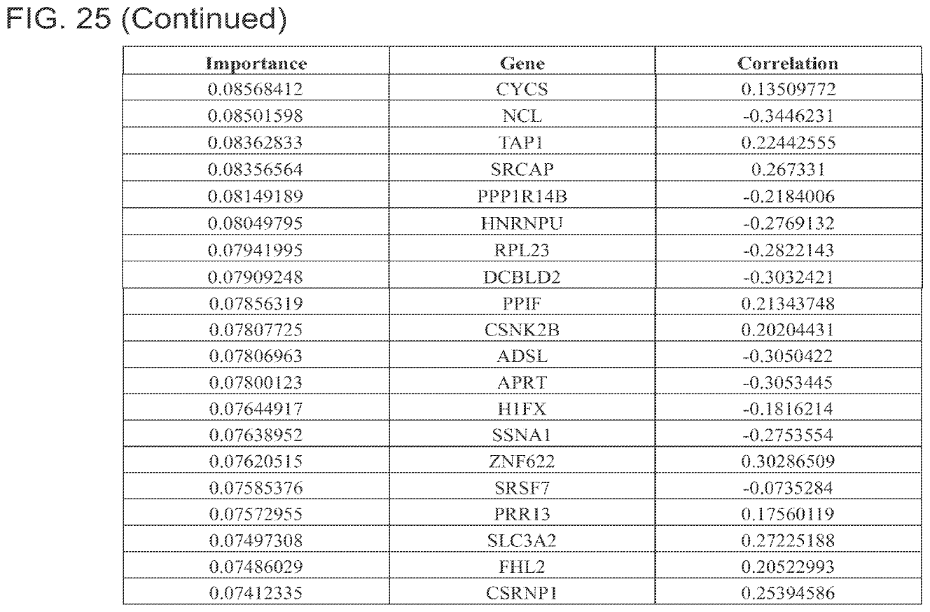

18. The method of claim 1, wherein:
the test drug is selected for administration to the subject if about 25% or more of the single cell transcriptional profiles obtained from the population of cells from the subject match the reference test drug-responsive transcriptional signature;
the one or more single cell transcriptional profiles of the population of cells from the subject are obtained via next-generation sequencing, optionally via a Seq-Well, and/or Drop-Seq process;
the one or more single cell transcriptional profiles of the population of cells from the subject are obtained via a focused assay, optionally via a barcoding technology that enables spatially resolved, digital readouts of proteins and/or RNA targets in multiplexed assays and/or via an in situ hybridization approach;
the reference test drug-responsive transcriptional signature indicates diminished cell viability and/or apoptosis;
the predictive accuracy of early (e.g., 3-48 hours after biopsy and/or drug administration) single cell transcript profiles obtained from known drug-responsive cells is compared with the predictive accuracy of longer term (either post-drug administration or after isolation/expansion of a biopsy-derived cell line and/or organoid) transcript signatures of known drug-responsive cells, optionally wherein the early single cell transcript profiles are approximately as accurate as, or are more accurate than, the longer term transcript signatures of known drug-responsive cells;
steps (a)-(e) accurately predict whether the subject is responsive to the selected test drug;
the reference test drug-responsive transcriptional signature comprises two or more transcripts encoded by genes selected from FIG. 25, optionally three or more transcripts selected from FIG. 25, optionally four or more transcripts selected from FIG. 25, optionally five or more transcripts selected from FIG. 25, optionally six or more transcripts selected from FIG. 25, optionally seven or more transcripts selected from FIG. 25, optionally eight or more transcripts selected from FIG. 25, optionally nine or more transcripts selected from FIG. 25, optionally ten or more transcripts selected from FIG. 25, optionally twenty or more transcripts selected from FIG. 25, optionally thirty or more transcripts selected from FIG. 25, optionally forty or more transcripts selected from FIG. 25, optionally fifty or more transcripts selected from FIG. 25, optionally all transcripts of FIG. 25; and/or
the reference test drug-responsiveness transcriptional signature comprises gene expression values/measurements for one or more genes selected from the group consisting of YPEL5, SBDS, PMAIP1, CDKN1A, RASSF1, SERTAD1, PPP1R15A, RPS15, CCDC85B, MAFF, PTMA, MT.CYB, HBEGF, SESN2, HIST2H2AC, SAT1, PTP4A1, ZFAS1, FAM173A, SNHG15, MAT2A, ATF3, IL11, IL8, H3F3A, PDRG1, MRPL33, SRSF2, DDIT3 and NDUFB10, optionally relative to an appropriate control, optionally wherein the appropriate control comprises a gene expression level or measurement for a cell pre-determined not to be responsive to the test drug.

19-26. (canceled)

27. A method for identifying a transcriptional signature for drug-responsive cells, the method comprising:
a) contacting a population of cells comprising multiple cell types with a drug, wherein the multiple cell types are known to differ in their responsiveness to the drug;
b) obtaining single cell transcript sequences from multiple cells of the population of cells; and
c) comparing the single cell transcript sequences obtained from known drug-responsive cell types of the population to single cell transcript sequences obtained from known non-drug-responsive cell types of the population, thereby identifying transcript sequences that distinguish the known drug-responsive cell types from the known non-drug-responsive cell types; and
d) assembling the transcript sequences that distinguish the known drug-responsive cell types from the known non-drug-responsive cell types into a transcriptional signature for drug-responsive cells,
thereby identifying a transcriptional signature for drug-responsive cells.

28. The method of claim 27, wherein:
genetic profiling (e.g., SNP profiling) is employed to distinguish between the multiple cell types of the population of cells in comparing the single cell transcript sequences obtained from known drug-responsive cell types of the population to single cell transcript sequences obtained from known non-drug-responsive cell types of the population;
single cell transcript sequences are obtained at multiple timepoints after contacting the population of cells with the drug, optionally wherein the timepoints are between 3 and 48 hours after contacting the population of cells with the drug;
the population of cells comprising multiple cell types is obtained from cell lines and/or ex vivo organoid models;
the transcriptional signature for drug-responsive cells comprises two or more transcripts encoded by genes selected from FIG. 25, optionally three or more transcripts selected from FIG. 25, optionally four or more transcripts selected from FIG. 25, optionally five or more transcripts selected from FIG. 25, optionally six or more transcripts selected from FIG. 25, optionally seven or more transcripts selected from FIG. 25, optionally eight or more transcripts selected from FIG. 25, optionally nine or more transcripts selected from FIG. 25, optionally ten or more transcripts selected from FIG. 25, optionally twenty or more transcripts selected from FIG. 25, optionally thirty or more transcripts selected from FIG. 25, optionally forty or more transcripts selected from FIG. 25, optionally fifty or more transcripts selected from FIG. 25, optionally all transcripts of FIG. 25; and/or
the transcriptional signature for drug-responsive cells comprises gene expression values/measurements for one or more genes selected from the group consisting of YPEL5, SBDS, PMAIP1, CDKN1A, RASSF1, SERTAD1, PPP1R15A, RPS15, CCDC85B, MAFF, PTMA, MT.CYB, HBEGF, SESN2, HIST2H2AC, SAT1, PTP4A1, ZFAS1, FAM173A, SNHG15, MAT2A, ATF3, IL11, IL8, H3F3A, PDRG1, MRPL33, SRSF2, DDIT3 and NDUFB10, optionally relative to an appropriate control, optionally wherein the appropriate control comprises a gene expression level or measurement for a cell pre-determined not to be responsive to the test drug.

29-37. (canceled)
